(19) United States
(12) Patent Application Publication
(10) Pub. No.: US 2023/0146909 A1
(43) Pub. Date: May 11, 2023

(54) DYE INK COMPOSITION, BLACK DYE INK, INK JET RECORDING DYE INK, INK JET RECORDING METHOD, AND AQUEOUS DYE SOLUTION

(71) Applicant: FUJIFILM Corporation, Tokyo (JP)

(72) Inventor: Keiichi TATEISHI, Ashigarakami-Gun (JP)

(73) Assignee: FUJIFILM Corporation, Tokyo (JP)

(21) Appl. No.: 17/887,522

(22) Filed: Aug. 15, 2022

(30) Foreign Application Priority Data
Sep. 29, 2021 (JP) 2021-160151

(51) Int. Cl. C09D 11/328; C09B 47/22; C09D 11/38; C07F 1/08; C07D 251/54; B41M 5/00

(52) U.S. Cl. CPC C09D 11/328; C09B 47/22; C09D 11/38; C07F 1/08; C07D 251/54; B41M 5/0023

(57) ABSTRACT

A dye ink composition includes. a phthalocyanine compound represented by the General Formula (I), in which, in the General Formula (I), X₁, X₂, X₃, and X₄ each independently represent —N═N—Cp; Cp represents an organic group; Y₁, Y₂, Y₃, and Y₄ each independently represent a substituent; at least one of X₁, X₂, X₃, X₄, Y₁, Y₂, Y₃, or Y₄ contains an ionic hydrophilic group; a1, a2, a3, a4, b1, b2, b3, and b4 each independently represent an integer of 0 to 4; a1 +b1, a2+b2, a3+b3, and a4+b4 are each independently an integer of 0 to 4; all of a1, a2, a3, and a4 are not 0 at the same time; and M represents a hydrogen atom, a metal element, a metal oxide, a metal hydroxide, or a metal halide.

### CROSS-REFERENCE TO RELATED APPLICATIONS

This application is based on and claims priority under 35 USC 119 from Japanese Patent Application No. 2021-160151 filed on Sep. 29, 2021, the entire contents of which are incorporated herein by reference.

### BACKGROUND OF THE INVENTION

### 1. Field of the Invention

The present invention relates to a dye ink composition, a black dye ink, an ink jet recording dye ink, an ink jet recording method, and an aqueous dye solution.

### 2. Description of the Related Art

In the related art, a dye ink composition containing a phthalocyanine compound has been known. For example, JP3949385B and JP4145153B disclose dye ink compositions containing a phthalocyanine compound having a specific substituent.

In addition, Russian Journal of General Chemistry, 2018, Vol. 88, No. 6, p. 1164-1171, and Chemistry Letters Vol. 33, No. 11 (2004) p. 1450-451 disclose compounds having a structure in which a dialkylaminobenzene azo group is bonded to a skeleton of phthalocyanine.

### SUMMARY OF THE INVENTION

Dye ink compositions of various colors such as yellow, magenta, cyan, and black have been put into practical use as ink jet recording dye ink. However, the current situation is that no dye that fully satisfies various performances has been reported, especially for black.

An object of the present invention is to provide a dye ink composition that is capable of forming an image excellent in black color tone, printing density, and light resistance and is excellent in storage stability, a black dye ink containing the dye ink composition, an ink jet recording dye ink containing the dye ink composition or the black dye ink, an ink jet recording method using the ink jet recording dye ink, and an aqueous dye solution that can be used in the production of the dye ink composition.

That is, the inventors of the present invention found that the above object can be achieved by the following configurations.

[1] A dye ink composition comprising. a phthalocyanine compound represented by the following General Formula (I),

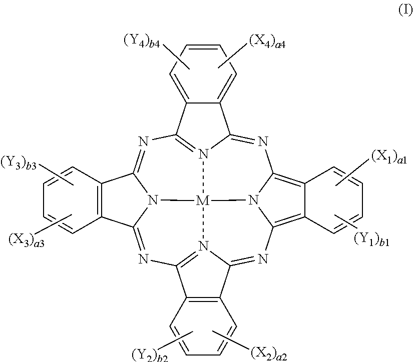

in which, in the General Formula (I),

X₁, X₂, X.;, and X₄ each independently represent —N═N—Cp,

Cp represents an organic group,

Y₁, Y₂, Y₃;, and Y₄ each independently represent a substituent, at least one of Xi, X_{2,} X₃,X₄, Y₁, Y₂, Y₃, or Y₄ contains an ionic hydrophilic group, a₁, a₂, a₃, a₄, b 1, b2, b3, and b4 each independently represent an integer of 0 to 4, a₁ +b 1, a₂+b2, a₃+b3, and a₄+b4 are each independently an integer of 0 to 4, all of a₁, a₂, a₃, and a₄ are not 0 at the same time, and

M represents a hydrogen atom, a metal element, a metal oxide, a metal hydroxide, or a metal halide.

[2] The dye ink composition according to [1], in which the phthalocyanine compound is a phthalocyanine compound represented by the following General Formula (II),

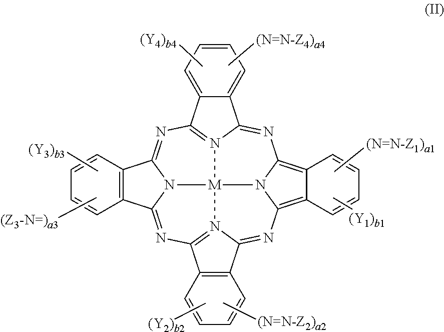

in which, in the General Formula (II),

Z1, Z2, Z3, and Z4 each independently represent a heterocyclic group, the heterocyclic group may have a substituent,

Y₁, Y₂, Y₃, and Y₄ each independently represent a substituent,

a₁, a₂, a₃, a₄, b1, b2, b3, and b4 each independently represent an integer of 0 to 4,

a1 +b 1, a₂+b2, a₃+b3, and a₄+b4 are each independently an integer of 0 to 4,

all of a₁, a₂, a₃, and a₄ are not 0 at the same time,

M represents a hydrogen atom, a metal element, a metal oxide, a metal hydroxide, or a metal halide, and

at least one of Z₁, Z₂, Z₃, Z₄, Y₁, Y₂, Y₃, or Y₄ contains an ionic hydrophilic group.

[3] The dye ink composition according to [2], in which the phthalocyanine compound is a phthalocyanine compound represented by the following General Formula (III),

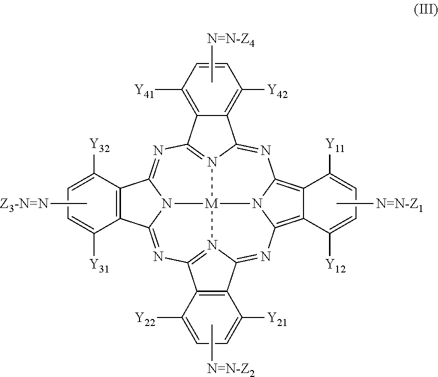

in which, in the General Formula (III),

Z₁, Z₂, Z₃, and Z₄ each independently represent a heterocyclic group, the heterocyclic group may have a substituent,

M represents a hydrogen atom, a metal element, a metal oxide, a metal hydroxide, or a metal halide,

Y₁₁, Y₁₂, Y₂₁, Y₂₂, Y₃₁, Y₃₂, Y₄₁ and Y₄₂ each independently represent a hydrogen atom or a substituent, and

at least one of Z₁, Z₂, Z₃, Z₄, Yu, Y₁₂, Y_{21,} ^{Y22,} Y₃₁, Y₃₂, Y₄₁, or Y₄₂ contains an ionic hydrophilic group.

[4] The dye ink composition according to [3], in which the phthalocyanine compound is a phthalocyanine compound represented by the following General Formula (IV),

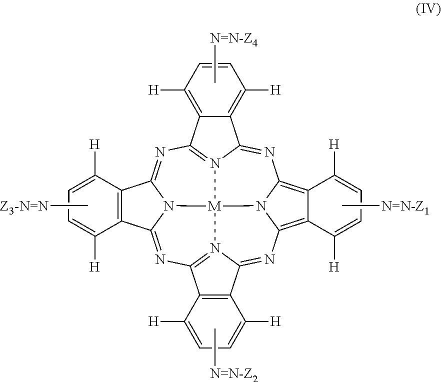

in which, in the General Formula (IV),

Z₁, Z₂, Z₃, and Z₄ each independently represent a heterocyclic group, the heterocyclic group may have a substituent,

M represents a hydrogen atom, a metal element, a metal oxide, a metal hydroxide, or a metal halide, and

at least one of Z₁, Z₂, Z₃, or Z₄ contains an ionic hydrophilic group.

[5] The dye ink composition according to any one of [1] to [4],

in which the ionic hydrophilic group is at least one of -SO3R, -CO2R, or —PO(OR)2,

R represents a hydrogen atom or a counter cation, and

two R's of -PO(OR)2 may be the same or different from each other.

[6] The dye ink composition according to any one of [1] to [5], in which a content of the phthalocyanine compound is 1.0% to 6.5°/o by mass with respect to a total mass of the dye ink composition.

[7] The dye ink composition according to any one of [1] to [6], further comprising: a compound represented by the following General Formula (P),

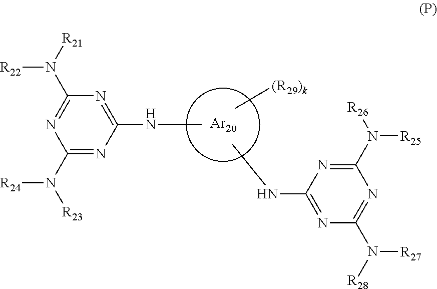

in which, in the General Formula (P), Arlo represents a benzene ring or a naphthalene ring; R₂₁ to R₂₈ each independently represent a hydrogen atom or a substituent; Rn and Rn may be bonded to each other to form a ring; R₂₃ and R₂₄ may be bonded to each other to form a ring; Res and R₂₆ may be bonded to each other to form a ring; Rn and R₂₈ may be bonded to each other to form a ring; R₂₉ represents a substituent; in a case where Arlo represents a benzene ring, k represents an integer of 0 to 4; in a case where Arlo represents a naphthalene ring, k represents an integer of 0 to 6; in a case where a plurality of Ray's are present, the plurality of R₂₉'s may be the same or different from each other; in a case where a plurality of R₂₉'s are present, the plurality of R₂₉'s may be bonded to each other to form a ring; and at least one of Rn to R₂₉ has a hydrophilic group.

[8] The dye ink composition according to [7], in which a content of the compound represented by the General Formula (P) is 0.5% to 3.0% by mass with respect to a total mass of the dye ink composition.

[9] The dye ink composition according to any one of [1] to [8], further comprising a toning dye.

[10] The dye ink composition according to any one of [1] to [9], further comprising a chelating agent.

[11] The dye ink composition according to any one of [1] to [10], further comprising a preservative.

[12] A black dye ink comprising the dye ink composition according to any one of [1]to [11].

[13] An ink jet recording dye ink comprising:
the dye ink composition according to any one of [1] to [11].

[14] An ink jet recording dye ink comprising:
the black dye ink according to [12]

[15] An ink jet recording method comprising jetting the ink jet recording dye ink according to [13] using an ink jet method-type recording head.

[16] An ink jet recording method comprising jetting the ink jet recording dye ink according to [14] using an ink jet method-type recording head.

[17] An aqueous dye solution comprising.

a preservative,

in which the aqueous dye solution contains a phthalocyanine compound represented by the following General Formula (1), and

a content of the phthalocyanine compound represented by the General Formula (I) is 8% to 15% by mass with respect to a total mass of the aqueous dye solution,

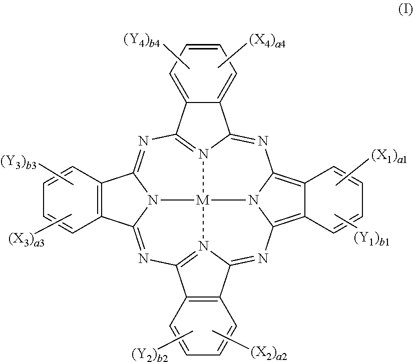

in which, in the General Formula (1),
X₁, X₂, X₃, and X₄ each independently represent —N═N—Cp,

Cp represents an organic group,

Y₁, Y₂, Y₃, and Y₄ each independently represent a substituent,

at least one of Xi, X₂, X_{3,} ^{X4,} Y₁, Y₂, Y₃, or Y₄ contains an ionic hydrophilic group,

a₁, a₂, a₃, a₄, b1, b2, b3, and b4 each independently represent an integer of 0 to 4,

a₁ +b1, a₂+b2, a₃+b3, and a₄+b4 are each independently an integer of 0 to 4,

all of a₁, a₂, a₃, and a₄ are not 0 at the same time, and

M represents a hydrogen atom, a metal element, a metal oxide, a metal hydroxide, or a metal halide.

[18] The aqueous dye solution according to [17], further comprising a toning dye.

According to the present invention, it is possible to provide a dye ink composition that is capable of forming an image excellent in black color tone, printing density, and light resistance and is excellent in storage stability, a black dye ink containing the dye ink composition, an ink jet recording dye ink containing the dye ink composition or the black dye ink, an ink jet recording method using the ink jet recording dye ink, and an aqueous dye solution that can be used in the production of the dye ink composition.

### DESCRIPTION OF THE PREFERRED EMBODIMENTS

Hereinafter, the present invention will be described in detail with reference to preferred embodiments.

### Dye Ink Composition

The dye ink composition according to the embodiment of the present invention is a dye ink composition containing a phthalocyanine compound represented by the following General Formula (I).

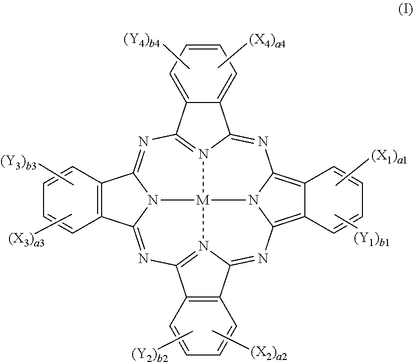

In the General Formula (I),

X₁, X₂, X₃, and X₄ each independently represent —N═N—Cp,

Cp represents an organic group,

Y₁, Y₂, Y₃, and Y₄ each independently represent a substituent,

at least one of Xi, X₂, X₃, X₄, Yt, Y₂, Y₃, or Y₄ contains an ionic hydrophilic group,

a1, a2, a3, a4, b1, b2, b3, and b4 each independently represent an integer of 0 to 4,

a1 +b1, a₂+b2, a₃+b3, and a₄+b4 are each independently an integer of 0 to 4,

all of a₁, a₂, a₃, and a₄ are not 0 at the same time, and

M represents a hydrogen atom, a metal element, a metal oxide, a metal hydroxide, or a metal halide.

The reason why the dye ink composition according to the embodiment of the present invention is capable of forming an image excellent in black color tone, printing density, and light resistance and is excellent in storage stability has not been completely revealed; however, the inventors of the present invention presume as follows.

Phthalocyanine compounds and azo compounds have been widely proposed as coloring agents that cover the tone in the visible range, particularly in a long wavelength range of 600 nm or more. Although the phthalocyanine compound is excellent in cyan tone, it cannot cover the tone in a short wavelength range of 400 nm to 600 nm in a case of being applied to a use application for black tone. On the other hand, in azo compounds, there are compound having black tone that covers the entire visible range by linking an azo group with a bis-type, a tris-type, and tetrakis-type to extend a conjugated system that becomes a chromophore+an auxochrome. However, the image formation excellent in storage stability and light resistance has not been satisfactorily achieved at a high level.

The inventors of the present invention carried out diligent studies and have developed a compound that balancedly achieves the storage stability, good toning of black tone, and storability of a formed image at a high level by directly and independently linking azo groups having a specific structure to each of the four fused moieties (the conjugated system) of a phthalocyanine compound and controlling the association property of the water-soluble phthalocyanine (the ability to form molecular aggregates). It is conceived that the dye ink composition according to the embodiment of the present invention is capable of forming an image excellent in black color tone, printing density, and light resistance and is excellent in storage stability due to the matters described in the following (1) to (3).

(1) The application of the molecular design (the introduction of a specific number of specific substituents to a characteristic substitution position) that controls the main absorption in the visible absorption range so that the wavelength range having high visual sensitivity (for example, from a Amax of 670 to 680 nm to a λmax 600 to 640 nm) can be covered, by using association characteristics of the water-soluble phthalocyanine compound (the association mode of molecular aggregates shifts to a short wavelength side by optimizing the slip angle between molecules. commonly known as an H association product).

(2) The improvement of the oxidation potential by introducing an azo group into the four electronically noble fused moieties for the purpose of imparting resistance to the oxidation reaction due to the attack of oxygen or ozone gas on the phthalocyanine compound (the stabilization of the highest occupied molecular orbital (HOMO) level).

(3) The selection of the substituent having high oxidation potential (low HOMO) level and high lowest unoccupied molecular orbital (LUMO) level for the azo coloring agent mother nucleus covering the short wavelength range, for the purpose of imparting the stability and image fastness of the ink solution.

In the present invention, in a case where the compound is a salt, the salt is present in a water-soluble ink in a state of being completely dissolved into ions. In a case where it has an ionic hydrophilic group having a high acid dissociation constant (pKa), it may be present in a state where most thereof is dissociated and a part thereof is dissolved in a salt state.

Phthalocyanine compound represented by General Formula (l) A phthalocyanine compound represented by General Formula (I) is typically used as the coloring agent.

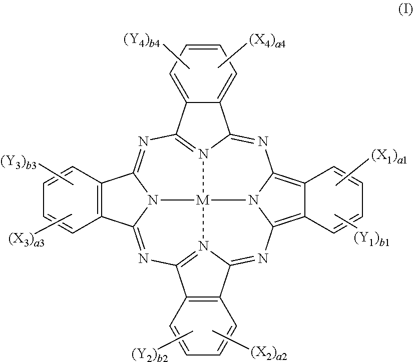

In General Formula (I), Xi, X₂, X.;, and X. each independently represent —N═N—Cp.

Cp represents an organic group,

Examples of the organic group represented by Cp include an organic residue of a coupling component (an organic residue of a component capable of forming an azo coloring agent by a chemical reaction).

Cp is preferably an organic residue of a coupling component capable of forming an azo coloring agent in (1) a coupling reaction with a diazonium salt or (2) an addition reaction of an oxidant of a hydrazine derivative, which is generally widely used.

Examples of Cp include a heterocyclic group, an aromatic hydrocarbon group, and a group obtained by removing one hydrogen atom from a compound having an active methylene group. These groups may have one or more substituents in a case of being allowed.

Examples of the substituent include the groups described as the substituents represented by Y₁, Y₂, Y₃, and Y₄ described later. The substituent is preferably an amino group, an alkylamino group, an arylamino group, an alkylcarbonylamino group, an arylcarbonylamino group, a heterocycliccarbonylamino group, a ureido group, an aminosulfonylamino group, an alkyloxycarbonylamino group, an aryloxycarbonylamino group, an alkylsulfonylamino group, a heterocyclicoxycarbonylamino group, an arylsulfonylamino group, a heterocyclicsulfonylamino group, a cyano group, an alkyl group, a hydroxy group, an ionic hydrophilic group, or the like.

The heterocyclic group represented by Cp is preferably an aromatic heterocyclic group or a non-aromatic heterocyclic group, and more preferably an aromatic heterocyclic group.

In a case where Cp represents an aromatic heterocyclic group (a heteroaryl group), it is preferably an aromatic heterocyclic group containing at least one heteroatom selected from the group consisting of a nitrogen atom, a sulfur atom, and an oxygen atom, and more preferably an aromatic heterocyclic group containing at least one heteroatom selected from the group consisting of a nitrogen atom and sulfur atoms. It is still more preferably an aromatic heterocyclic group containing one or more nitrogen atoms in terms of toning of tone and image fastness.

Examples of the aromatic heterocyclic group include a group obtained by removing one hydrogen atom from a 5-membered ring aromatic heterocyclic compound such as pyrrole, imidazole, pyrazole, oxazole, isooxazole, thiazole, isothiazole, triazole, or thiophene, and a group obtained by removing one hydrogen atom from a 6-membered ring aromatic heterocyclic compound such as pyridine, pyrazine, pyrimidine, pyridazine, triazine, thiazine, and oxazine.

Further, the aromatic heterocyclic group may be a group obtained by removing one hydrogen atom from a compound (for example, indole, quinoline, or isoquinoline) in which the 5-membered ring aromatic heterocyclic compound or the 6-membered ring aromatic heterocyclic compound is fused with at least one selected from the group consisting of the 5-membered ring aromatic heterocyclic compound, the 6-membered ring aromatic heterocyclic compound, an aromatic hydrocarbon (for example, benzene or naphthalene), a cycloalkane (for example, cyclopentane or cyclohexane), and a non-aromatic heterocyclic compound (for example, a 5-membered non-aromatic heterocyclic compound or a 6-membered non-aromatic heterocyclic compound described later).

The aromatic heterocyclic group may have a substituent.

A carbon atom contained as a ring member in the aromatic heterocyclic group may be substituted with an oxo group (=O).

Specifically, the aromatic heterocyclic group is preferably a group obtained by removing one hydrogen atom from aminopyridine, aminopyrazine, aminopyridazine, aminopyrimidine, aminothiazole, aminopyrrole, 5-aminopyrazole, aminothiophene, or aminooxazol, more preferably a group obtained by removing one hydrogen atom from aminopyridine, aminopyrazi ne, aminopyridazine, aminopyrimidine, aminothiazole, aminopyrrole, 5-aminopyrazole, or aminothiophene, and still more preferably a group obtained by removing one hydrogen atom from aminopyridine, aminopyrazine, aminopyridazine, aminopyrimidine, aminothiazole, aminopyrrole, or 5-aminopyrazole.

In a case where Cp represents a non-aromatic heterocyclic group (an aliphatic heterocyclic group), it is preferably a non-aromatic heterocyclic group containing at least one heteroatom selected from the group consisting of a nitrogen atom, a sulfur atom, and an oxygen atom, more preferably a non-aromatic heterocyclic group containing at least one heteroatom selected from the group consisting of a nitrogen atom and sulfur atoms, and still more preferably a non-aromatic heterocyclic group containing one or more nitrogen atoms.

Examples of the non-aromatic heterocyclic group include a group obtained by removing one hydrogen atom from a 5-membered ring non-aromatic heterocyclic compound such as pyrrolidine, pyrroline, or 2-oxazolidone, and a group obtained by removing one hydrogen atom from a 6-membered ring-non-aromatic heterocyclic compound such as morpholine, piperizine, or piperazine.

Further, the non-aromatic heterocyclic group may be a group obtained by removing one hydrogen atom from a compound in which the 5-membered ring non-aromatic heterocyclic compound or the 6-membered ring non-aromatic heterocyclic compound is fused with at least one selected from the group consisting of the 5-membered ring non-aromatic heterocyclic compound, the 6-membered ring non-aromatic heterocyclic compound, and a cycloalkane (for example, cyclopentane or cyclohexane).

The non-aromatic heterocyclic group may have a substituent.

A carbon atom contained as a ring member in the non-aromatic heterocyclic group may be substituted with an oxo group (=O).

In a case where Cp represents an aromatic hydrocarbon group (an aryl group), it may be a monocyclic aryl group or a polycyclic aryl group.

The aryl group represented by Cp is preferably an aryl group having 6 to 20 carbon atoms, more preferably an aryl group having 6 to 10 carbon atoms, and examples thereof include a phenyl group, a naphthyl group, and an anthryl group.

The aryl group represented by Cp may have a substituent.

The aryl group represented by Cp preferably has at least one substituent selected from the group consisting of an amino group, an alkylamino group, an alkylcarbonylamino group, a ureido group, an aminosulfonylamino group, an alkyloxycarbonylamino group, an alkylsulfonylamino group, a cyano group, an alkyl group, a hydroxy group, and an ionic hydrophilic group.

Specifically, the aryl group represented by Cp is preferably aniline, phenol, naphthol, or aminonaphthalene.

In a case where Cp represents a group obtained by removing one hydrogen atom from a compound having an active methylene group, specific examples of the compound having an active methylene group include an acylacetic acid ester, an acylacetic acid amide, a cyanoacetic acid ester, a cyanoacetic acid amide, malonic acid, a malonic acid ester, a malonic acid amide, 5-pyrrozolone, barbituric acid, pyrazolidinedione, dimedone, and hydroxycoumarin. These groups may have a substituent.

Cp preferably represents a heterocyclic group or an aromatic hydrocarbon group, and it more preferably represents a heterocyclic group.

In General Formula (I), Y₁, Y₂, Y₃, and Y₄ each independently represent a substituent.

The substituent represented by Y1, Y₂, Y₃, and Y₄ is also referred to as a “substituent Y“.

The substituent Y preferably represents a halogen atom, an alkyl group, a cycloalkyl group, an alkenyl group, an aralkyl group, an aryl group, a heterocyclic group, a cyano group, a hydroxy group, a nitro group, an amino group, an alkylamino group, an arylamino group, an alkylcarbonylamino group, an arylcarbonylamino group, a heterocycliccarbonylamino group, a ureido group, an aminosulfonylamino group, an alkyloxycarbonylamino group, an aryloxycarbonylamino group, an alkylsulfonylamino group, a heterocyclicoxycarbonylamino group, an arylsulfonylamino group, a heterocyclicsulfonylamino group, an alkyloxy group, an aryloxy group, a heterocyclicoxy group, an acyloxy group, an carbamoyloxy group, an silyloxy group, an alkylthio group, an arylthio group, a heterocyclicthio group, an aminocarbonyl group, an alkyloxycarbonyl group, an aryloxycarbonyl group, a heterocyclicoxycarbonyl group, an alkylthiocarbonyl group, an arylthiocarbonyl group, a heterocyclicthiocarbonyl group, an acyl group, an aminosulfonyl group, an imide group, a phosphoryl group, or ionic hydrophilic group.

The substituent Y may further have a substituent in a case of being allowed.

Examples of the halogen atom represented by the substituent Y include a fluorine atom, a chlorine atom, and a bromine atom.

The alkyl group represented by the substituent Y includes an alkyl group having a substituent and an unsubstituted alkyl group. The alkyl group is preferably an alkyl group having 1 to 12 carbon atoms in terms of the number of carbon atoms in a case where the substituent is removed. Examples of the substituent include a hydroxy group, an alkoxy group, a cyano group, a halogen atom, and an ionic hydrophilic group. Examples of the alkyl group include a methyl group, an ethyl group, an n-propyl group, an i-propyl group, an n-butyl group, an isobutyl group, a sec-butyl group, a t-butyl group, an n-pentyl group, a hydroxyethyl group, a methoxyethyl group, a cyanoethyl group, a trifluoromethyl group, a 3-sulfopropyl group, and a 4-sulfobutyl group.

The cycloalkyl group represented by the substituent Y includes a cycloalkyl group having a substituent and an unsubstituted cycloalkyl group. The cycloalkyl group is preferably a cycloalkyl group having 5 to 12 carbon atoms in terms of the number of carbon atoms in a case where the substituent is removed. Examples of the substituent include an ionic hydrophilic group. Examples of the cycloalkyl group include a cyclohexyl group.

The alkenyl group represented by the substituent Y includes an alkenyl group having a substituent and an unsubstituted alkenyl group. The alkenyl group is preferably an alkenyl group having 2 to 12 carbon atoms in terms of the number of carbon atoms in a case where the substituent is removed. Examples of the substituent include an ionic hydrophilic group. Examples of the alkenyl group include a vinyl group and an allyl group.

The aralkyl group represented by the substituent Y includes an aralkyl group having a substituent and an unsubstituted aralkyl group. The aralkyl group is preferably an aralkyl group having 7 to 12 carbon atoms in terms of the number of carbon atoms in a case where the substituent is removed. Examples of the substituent include an ionic hydrophilic group. Examples of the aralkyl group include a benzyl group and a 2-phenethyl group.

The aryl group represented by the substituent Y includes an aryl group having a substituent and an unsubstituted aryl group. The aryl group is preferably an aryl group having 6 to 12 carbon atoms in terms of the number of carbon atoms in a case where the substituent is removed. Examples of the substituent include an alkyl group, an alkoxy group, a halogen atom, an alkylamino group, and an ionic hydrophilic group. Examples of the aryl group include a phenyl group, a p-tolyl group, a p-methoxyphenyl group, an o-chlorophenyl group, and an m-(3-sulfopropylamino)phenyl group.

The heterocyclic group represented by the substituent Y includes a heterocyclic group having a substituent and an unsubstituted heterocyclic group. The heterocyclic group is preferably a 5-membered or 6-membered heterocyclic group. It is also preferably a heterocyclic group in which the 5-membered or 6-membered heterocyclic ring forms a fused ring together with another ring (for example, a 5-membered or 6-membered heterocyclic ring or a 5-membered or 6-membered carbon ring). The heterocyclic group may be an aromatic heterocyclic group or a non-aromatic heterocyclic group. The heteroatom contained in the heterocyclic group is not particularly limited; however, examples thereof include a sulfur atom, a nitrogen atom, and an oxygen atom. Examples of the substituent include an ionic hydrophilic group. Examples of the heterocyclic group include a 2-pyridyl group, a 2-thienyl group, and a 2-furyl group.

The alkylamino group represented by the substituent Y includes an alkylamino group having a substituent and an unsubstituted alkylamino group. The alkylamino group is preferably an alkylamino group having 1 to 6 carbon atoms in terms of the number of carbon atoms in a case where the substituent is removed. Examples of the substituent include an ionic hydrophilic group. Examples of the alkylamino group include a methylamino group and a diethylamino group.

The arylamino group represented by the substituent Y includes an arylamino group having a substituent and an unsubstituted arylamino group. The arylamino group is preferably an arylamino group having 6 to 12 carbon atoms in terms of the number of carbon atoms in a case where the substituent is removed. Examples of the substituent include a halogen atom and an ionic hydrophilic group. Examples of the arylamino group include an anilino group and a 2-chloroanilino group.

The alkylcarbonylamino group represented by the substituent Y includes an alkylcarbonylamino group having a substituent and an unsubstituted alkylcarbonylamino group. The alkylcarbonylamino group is preferably an alkylcarbonylamino group having 2 to 12 carbon atoms in terms of the number of carbon atoms in a case where the substituent is removed. Examples of the substituent include an ionic hydrophilic group. Examples of the alkylcarbonylamino group include an ethylcarbonylamino group, a carboxyethylcarbonylamino group, and a salt thereof.

The arylcarbonylamino group represented by the substituent Y includes an arylcarbonylamino group having a substituent and an unsubstituted arylcarbonylamino group. The arylcarbonylamino group is preferably an arylcarbonylamino group having 7 to 12 carbon atoms in terms of the number of carbon atoms in a case where the substituent is removed. Examples of the substituent include an ionic hydrophilic group. Examples of the arylcarbonylamino group include a phenylcarbonylamino group, a 3-sulfo-benzenecarbonylamino group, and a salt thereof.

The heterocycliccarbonylamino group represented by the substituent Y includes a heterocycliccarbonylamino group having a substituent and an unsubstituted heterocycliccarbonylamino group. The heterocyclic group in the heterocycliccarbonylamino group is preferably a 5-membered or 6-membered heterocyclic group. It is also preferably a heterocyclic group in which the 5-membered or 6-membered heterocyclic ring forms a fused ring together with another ring (for example, a 5-membered or 6-membered heterocyclic ring or a 5-membered or 6-membered carbon ring). The heterocyclic group may be an aromatic heterocyclic group or a non-aromatic heterocyclic group. The heteroatom contained in the heterocyclic group is not particularly limited; however, examples thereof include a sulfur atom, a nitrogen atom, and an oxygen atom. Examples of the substituent include an ionic hydrophilic group. Examples of the heterocycliccarbonylamino group include a 2-pyridylcarbonylamino group, a 2-thienylcarbonylamino group, and a 2-furylcarbonylamino group.

The ureido group represented by the substituent Y includes a ureido group having a substituent and an unsubstituted ureido group. The ureido group is preferably a ureido group having 1 to 12 carbon atoms in terms of the number of carbon atoms in a case where the substituent is removed. Examples of the substituent include an alkyl group and an aryl group. Examples of the ureido group include a 3-methylureido group, a 3,3-dimethylureido group, and a 3-phenylureido group.

The aminosulfonylamino group represented by the substituent Y includes an aminosulfonylamino group having a substituent and an unsubstituted aminosulfonylamino group. Examples of the substituent include an alkyl group. Examples of the aminosulfonylamino group include an N,N-dipropylaminosulfonylamino group.

The alkyloxycarbonylamino group represented by the substituent Y includes an alkyloxycarbonylamino group having a substituent and an unsubstituted alkyloxycarbonylamino group. The alkyloxycarbonylamino group is preferably an alkyloxycarbonylamino group having 2 to 12 carbon atoms in terms of the number of carbon atoms in a case where the substituent is removed. Examples of the substituent include an ionic hydrophilic group. Examples of the alkyloxycarbonylamino group include an ethyloxycarbonylamino group, a sulfoethyloxycarbonylamino group, and a salt thereof.

The alkylsulfonylamino group represented by the substituent Y includes an alkylsulfonylamino group having a substituent and an unsubstituted alkylsulfonylamino group. The alkylsulfonylamino group is preferably an alkylsulfonylamino group having 1 to 12 carbon atoms in terms of the number of carbon atoms in a case where the substituent is removed. Examples of the substituent include an ionic hydrophilic group. Examples of the alkylsulfonylamino group include a methylsulfonylamino group.

The arylsulfonylamino group represented by the substituent Y includes an arylsulfonylamino group having a substituent and an unsubstituted arylsulfonylamino group. The arylsulfonylamino group is preferably an arylsulfonylamino group having 6 to 12 carbon atoms in terms of the number of carbon atoms in a case where the substituent is removed. Examples of the substituent include an ionic hydrophilic group. Examples of the arylsulfonylamino group include a phenylsulfonylamino group, a 3-carboxybenzenesulfonylamino group, and a salt thereof.

The heterocyclicsulfonylamino group represented by the substituent Y includes a heterocyclicsulfonylamino group having a substituent and an unsubstituted heterocyclicsulfonylamino group. The heterocyclic group in the heterocyclicsulfonylamino group is preferably a 5-membered or 6-membered heterocyclic group. It is also preferably a heterocyclic group in which the 5-membered or 6-membered heterocyclic ring forms a fused ring together with another ring (for example, a 5-membered or 6-membered heterocyclic ring or a 5-membered or 6-membered carbon ring). The heterocyclic group may be an aromatic heterocyclic group or a non-aromatic heterocyclic group. The heteroatom contained in the heterocyclic group is not particularly limited; however, examples thereof include a sulfur atom, a nitrogen atom, and an oxygen atom. Examples of the substituent include an ionic hydrophilic group. Examples of the heterocyclicsulfonylamino group include a 2-pyridylsulfonylamino group, a 2-thienylsulfonylamino group, and a 2-furylsulfonylamino group.

The aryloxycarbonylamino group represented by the substituent Y includes an aryloxycarbonylamino group having a substituent and an unsubstituted aryloxycarbonylamino group. The aryloxycarbonylamino group is preferably an aryloxycarbonylamino group having 7 to 12 carbon atoms in terms of the number of carbon atoms in a case where the substituent is removed. Examples of the substituent include an ionic hydrophilic group. Examples of the aryloxycarbonylamino group include a phenoxycarbonylamino group.

The alkyloxy group represented by the substituent Y includes an alkyloxy group having a substituent and an unsubstituted alkyloxy group. The alkyloxy group is preferably an alkyloxy group having 1 to 12 carbon atoms in terms of the number of carbon atoms in a case where the substituent is removed. Examples of the substituent include an alkyloxy group, a hydroxy group, and an ionic hydrophilic group. Examples of the alkyloxy group include a methyloxy group, an ethyloxy group, an isopropyloxy group, a methyloxyethyloxy group, a hydroxyethyloxy group, and a 3-carboxypropyloxy group.

The aryloxy group represented by the substituent Y includes an aryloxy group having a substituent and an unsubstituted aryloxy group. The aryloxy group is preferably an aryloxy group having 6 to 12 carbon atoms in terms of the number of carbon atoms in a case where the substituent is removed. Examples of the substituent include an alkoxy group and an ionic hydrophilic group. Examples of the aryloxy group include a phenoxy group, a p-methoxyphenoxy group, and an o-methoxyphenoxy group.

The heterocyclicoxy group represented by the substituent Y includes a heterocyclicoxy group having a substituent and an unsubstituted heterocyclicoxy group. The heterocyclic group in the heterocyclicoxy group is preferably a 5-membered or 6-membered heterocyclic group. It is also preferably a heterocyclic group in which the 5-membered or 6-membered heterocyclic ring forms a fused ring together with another ring (for example, a 5-membered or 6-membered heterocyclic ring or a 5-membered or 6-membered carbon ring). The heterocyclic group may be an aromatic heterocyclic group or a non-aromatic heterocyclic group. The heteroatom contained in the heterocyclic group is not particularly limited; however, examples thereof include a sulfur atom, a nitrogen atom, and an oxygen atom. Examples of the substituent include a hydroxy group and an ionic hydrophilic group. Examples of the heterocyclicoxy group include a 2-tetrahydropyranyloxy group.

The acyloxy group represented by the substituent Y includes an acyloxy group having a substituent and an unsubstituted acyloxy group. The acyloxy group is preferably an acyloxy group having 1 to 12 carbon atoms in terms of the number of carbon atoms in a case where the substituent is removed. Examples of the substituent include an ionic hydrophilic group. Examples of the acyloxy group include an acetoxy group and a benzoyloxy group.

The aminocarbonyloxy group represented by the substituent Y includes an aminocarbonyloxy group having a substituent and an unsubstituted aminocarbonyloxy group. Examples of the substituent include an alkyl group. Examples of the aminocarbonyloxy group include an N-methylcarbamoyloxy group.

The silyloxy group represented by the substituent Y includes a silyloxy group having a substituent and an unsubstituted silyloxy group. Examples of the substituent include an alkyl group. Examples of the silyloxy group include a trimethylsilyloxy group.

The alkylthio group represented by the substituent Y includes an alkylthio group having a substituent and an unsubstituted alkylthio group. The alkylthio group is preferably an alkylthio group having 1 to 12 carbon atoms in terms of the number of carbon atoms in a case where the substituent is removed. Examples of the substituent include an ionic hydrophilic group. Examples of the alkylthio group include a methylthio group and an ethylthio group.

The arylthio group represented by the substituent Y includes an arylthio group having a substituent and an unsubstituted arylthio group. The arylthio group is preferably an arylthio group having 6 to 12 carbon atoms in terms of the number of carbon atoms in a case where the substituent is removed. Examples of the substituent include an alkyl group and an ionic hydrophilic group. Examples of the arylthio group include a phenylthio group and a p-tolylthio group.

The heterocyclicthio group represented by the substituent Y includes a heterocyclicthio group having a substituent and an unsubstituted heterocyclicthio group. The heterocyclic group in the heterocyclicthio group is preferably a 5-membered or 6-membered heterocyclic group. It is also preferably a heterocyclic group in which the 5-membered or 6-membered heterocyclic ring forms a fused ring together with another ring (for example, a 5-membered or 6-membered heterocyclic ring or a 5-membered or 6-membered carbon ring). The heterocyclic group may be an aromatic heterocyclic group or a non-aromatic heterocyclic group. The heteroatom contained in the heterocyclic group is not particularly limited; however, examples thereof include a sulfur atom, a nitrogen atom, and an oxygen atom. Examples of the substituent include an ionic hydrophilic group. Examples of the heterocyclicthio group include a 2-pyridylthio group.

The aminocarbonyl group represented by the substituent Y includes an aminocarbonyl group having a substituent and an unsubstituted aminocarbonyl group. Examples of the substituent include an alkyl group. Examples of the aminocarbonyl group include a methylaminocarbonyl group and a dimethylaminocarbonyl group.

The alkyloxycarbonyl group represented by the substituent Y includes an alkyloxycarbonyl group having a substituent and an unsubstituted alkyloxycarbonyl group. The alkyloxycarbonyl group is preferably an alkyloxycarbonyl group having 2 to 12 carbon atoms in terms of the number of carbon atoms in a case where the substituent is removed. Examples of the substituent include an ionic hydrophilic group. Examples of the alkyloxycarbonyl group include a methyloxycarbonyl group and an ethyloxycarbonyl group.

The aryloxycarbonyl group represented by the substituent Y includes an aryloxycarbonyl group having a substituent and an unsubstituted aryloxycarbonyl group. The aryloxycarbonyl group is preferably an aryloxycarbonyl group having 7 to 12 carbon atoms in terms of the number of carbon atoms in a case where the substituent is removed. Examples of the substituent include an ionic hydrophilic group. Examples of the aryloxycarbonyl group include a phenoxycarbonyl group.

The acyl group represented by the substituent Y includes an acyl group having a substituent and an unsubstituted acyl group. The acyl group is preferably an acyl group having 1 to 12 carbon atoms in terms of the number of carbon atoms in a case where the substituent is removed. Examples of the substituent include an ionic hydrophilic group. Examples of the acyl group include an acetyl group and benzoyl group.

The aminosulfonyl group represented by the substituent Y includes an aminosulfonyl group having a substituent and an unsubstituted aminosulfonyl group. Examples of the substituent include an alkyl group and an aryl group. Examples of the aminosulfonyl group include a dimethylaminosulfonyl group, a di-(2-hydroxyethyl)aminosulfonyl group, and a phenylaminosulfonyl group.

The imide group represented by the substituent Y includes an imide group having a substituent and an unsubstituted imide group. The imide group is preferably represented by General Formula (K-1).

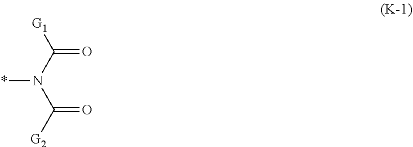

In General Formula (K-1), GI and G2 each independently represent a substituted or unsubstituted alkyl group. G₁ and G₂ may be bonded to form a ring. * represents a bonding position.

The description, specific examples, and preferred range of the alkyl group represented by G₁ and G₂ are the same as those of the alkyl group represented by the above-described substituent Y.

### Examples of the Imide Group Include an N-Phthalimide Group and an N-Succinimide Group

The phosphoryl group represented by the substituent Y includes a phosphoryl group having a substituent and an unsubstituted phosphoryl group. The phosphoryl group is preferably represented by General Formula (K-2).

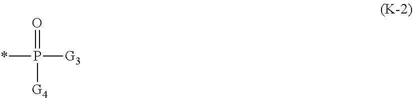

In General Formula (K-2), G₃ and G₄ are each independently represent a substituted or unsubstituted alkyl group, a substituted or unsubstituted alkoxy group, a substituted or unsubstituted aryl group, or a substituted or unsubstituted aryloxy group. G₃ and G₄ may be bonded to form a ring. * represents a bonding position.

The description, specific examples, and preferred range of the alkyl group, alkoxy group, aryl group, and aryloxy group represented by G₃ and G₄ are the same as those of the alkyl group, alkoxy group, aryl group, and aryloxy group represented by the above-described substituent Y, respectively.

Examples of the phosphoryl group include a diphenoxyphosphoryl group and a diphenylphosphoryl group.

The ionic hydrophilic group is a group selected from a sulfo group (—SO₃R), a carboxy group (—CO₂R), a thiocarboxy group, a sulfino group (—SO₂R), a phosphono group (-PO(OT)(OR), a dihydroxyphosphino group, a phosphate group (—PO(OR)2), a quaternary ammonium group, an acylsulfamoyl group (—SO₂N′R_{COT), a sulfonylcarbamoyl group (—CON}1R*SO₂-T), and a sulfonylaminosulfonyl group (—SO₂N′R+SO₂-T). The above R represents a hydrogen atom or a counter cation. Two R's of -PO(OR)2 may be the same or different from each other. The above T is a monovalent substituent (for example, an alkyl group or an aryl group).

From the viewpoint of imparting water solubility to the phthalocyanine compound and enhancing the storage stability of the dye ink composition, the ionic hydrophilic group is preferably an acidic group, more preferably at least one of a sulfo group (—SO₃R), a carboxy group (—CO₂R), or a phosphate group (—PO(OR)2), still more preferably a sulfo group (—SO₃R) or a carboxy group (—CO₂R), and most more preferably a sulfo group (—SO₃R).

The above R represents a hydrogen atom or a counter cation.

In a case where R represents a counter cation, examples thereof include an ammonium ion (NHa⁺), an alkali metal ion (for example, a lithium ion, a sodium ion, or a potassium ion), and an organic cation (for example, a tetramethylammonium ion, a tetramethylguanidinium ion, a tetramethylphosphonium ion).

R is preferably a hydrogen atom, an alkali metal ion, or an ammonium ion, and more preferably an alkali metal ion or a mixed ion of an alkali metal ion and an ammonium ion.

From the viewpoint of imparting water solubility to the phthalocyanine compound, R of —SO₃R is preferably a lithium ion, a sodium ion, a potassium ion, an ammonium ion, or a mixed ion of two or three thereof, more preferably a lithium ion, a sodium ion, or a mixed ion of a sodium ion and an ammonium ion, particularly preferably a lithium ion or a sodium ion, and most preferably a lithium ion.

From the viewpoint of imparting water solubility to the phthalocyanine compound, R of —CO₂R is preferably a lithium ion, a sodium ion, a potassium ion, an ammonium ion, or a mixed ion of two or three ions, more preferably a sodium ion, a potassium ion, or a mixed ion of a sodium ion and an ammonium ion, particularly preferably a sodium ion or a potassium ion, and most preferably a potassium ion.

It is noted that R may be a divalent counter cation. In a case where R is a divalent counter cation, it may have, for example, a form in which one R serves as a counter cation for two —SO₃⁻'s. From the viewpoint of water solubility, R is preferably a monovalent counter cation.

Y₁, Y₂, Y₃, and Y₄ in General Formula (I) preferably represent a halogen atom, an alkyl group, an aryl group, a cyano group, an alkyloxy group, a ureido group, an aminocarbonyl group, an aminosulfonyl group, or an alkyloxycarbonyl group, and more preferably represent a halogen atom or a cyano group.

Provided that at least one of Xi, X₂, X₃, Xi, Y₁, Y₂, Y₃, or Y₄ contains an ionic hydrophilic group. Here, the aspect containing an ionic hydrophilic group may be an aspect having an ionic hydrophilic group as a substituent or may be an aspect having, as a substituent, a group in which an ionic hydrophilic group is substituted with a group other than the ionic hydrophilic group. That is, the phthalocyanine compound represented by General Formula (I) has at least one ionic hydrophilic group.

M in General Formula (I) represents a hydrogen atom, a metal element, a metal oxide, a metal hydroxide, or a metal halide.

Examples of the metal element in M include Li, Na, K, Mg, Ti, Zr, V, Nb, Ta, Cr, Mo, W, Mn, Fe, Co, Ni, Ru, Rh, Pd, Os, Ir, Pt, Cu, Ag, Au, Zn, Cd, Hg, Al, Ga, In, Si, Ge, Sn, Pb, Sb, and Bi. Among these, Cu, Ni, Zn, or Al is particularly preferable, and Cu is most preferable.

Preferred examples of the metal oxide include VO and GeO.

Preferred examples of the metal hydroxide include Si(OH)2, Cr(OH)2, and Sn(OH)2.

Examples of the metal halide include AlCl, SiCl₂, VCl, VCl₂, VOCl1, FeCl, GaCl, and ZrCl.

M preferably represents a hydrogen atom or a metal element, more preferably represents a hydrogen atom, Cu, Al, Fe, Co, Ni, Zn, Mg, or Mn, and most preferably represents Cu.

In a case where M represents a hydrogen atom, the phthalocyanine compound represented by General Formula (I) is represented by General Formula (I-H).

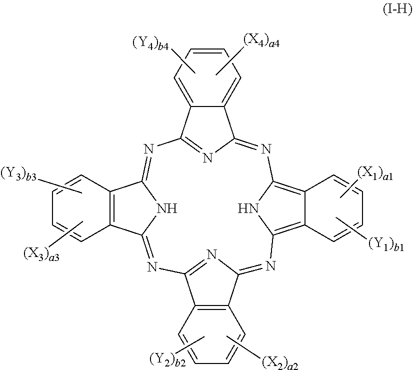

In General Formula (I-H), X₁, X₂, X₃, X₄, Y₁, Y₂, Y₃, Y₄, a₁, a₂, a₃, a₄, b1, b2, b3, and b4 respectively have the same meanings as those in General Formula (I).

a₁, a₂, a₃, a₄, b1, b2, b3, and b4 in General Formula (I) each independently represent an integer of 0 to 4. a₁ +b 1, a₂+b2, a₃+b3, and a₄+b4 are each independently an integer of 0 to 4, provided that all of a₁, a₂, a₃, and a₄ are not 0 at the same time, and, that is, a₁ +a₂+a₃+a₄ is an integer of 1 to 16.

a₁, a₂, a₃, and a₄ each independently represent preferably 1 or 2 and more preferably represent 1. b1, b2, b3, and b4 each independently represent preferably 0 or 1, and more preferably represent 0.

It is preferable that the ii-position of the phthalocyanine skeleton of General Formula (I) is substituted with X₁, X₂, Xi, and X₄. The a-position and 13-position of the phthalocyanine skeleton are as shown in General Formula (a) below.

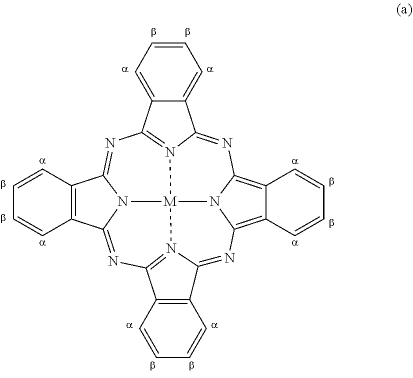

In General Formula (a), M has the same meaning as that in General Formula (I).

The phthalocyanine compound represented by the General Formula (I) is preferably a phthalocyanine compound represented by the following General Formula (II).

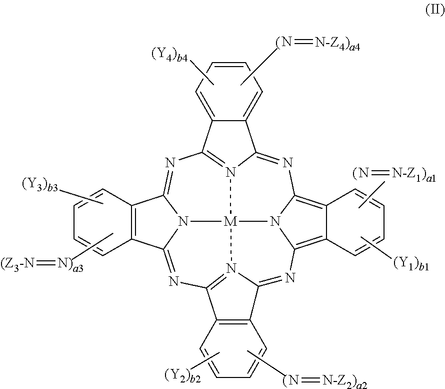

In the General Formula (II),

Z₁, Z₂, Z₃, and Z₄ each independently represent a heterocyclic group, the heterocyclic group may have a substituent, Y₁, Y₂, Y₃, and Y₄ each independently represent a substituent, a₁, a₂, a₃, a₄, b 1, b2, b3, and b4 each independently represent an integer of 0 to 4, a₁ +b 1, a₂+b2, a₃+b3, and a₄+b4 are each independently an integer of 0 to 4, all of a₁, a₂, a₃, and a₄ are not 0 at the same time, M represents a hydrogen atom, a metal element, a metal oxide, a metal hydroxide, or a metal halide, and

at least one of Z₁, Z₂, Z₃, Z₄, Y₁, Y₂, Y₃, or Y₄ contains an ionic hydrophilic group.

The description, specific examples, and preferred range of the heterocyclic group represented by Z₁, Z₂, Z₃, and Z₄ in General Formula (II) are the same as those described in the case where Cp in General Formula (I) represents a heterocyclic group.

Y₁, Y₂, Y₃, Y₄, a1, a2, a3, a4, b1, b2, b3, b4, and M respectively have the same meanings as those in General Formula (I), and the same applies to the description, specific examples, and preferred range thereof.

The phthalocyanine compound represented by the General Formula (I) or (II) is more preferably a phthalocyanine compound represented by the following General Formula (III).

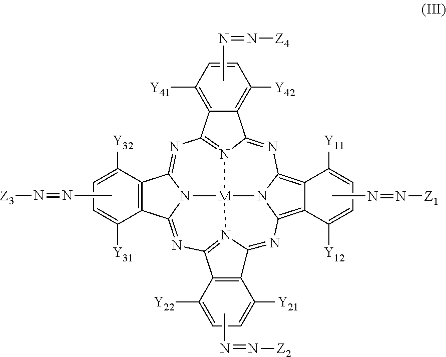

in the General Formula (III),

Z₁, Z₂, Z₃, and Z₄ each independently represent a heterocyclic group, the heterocyclic group may have a substituent,

M represents a hydrogen atom, a metal element, a metal oxide, a metal hydroxide, or a metal halide, and

Y₁₁, Y₁₂, Y₂₁, Y₁₂, Y₃₁, Y₃₂, Y₄₁ and Y₄₂ each independently represent a hydrogen atom or a substituent, and

at least one of Z₁, Z₂, Z₃, Za, ^{Y}₁₁, Y₁₂, Y_{21,} ^{Y}₂₂, Y₃₁, Y₃₂, Y₄₁, or Y₄₂ contains an ionic hydrophilic group.

The description, specific examples, and preferred range of the substituent represented by Y₁₁, Y₁₂, Y₂₁, Y₂₂, Y₃₁, Y₃₂, Y₄₁, and Y₄₂ in General Formula (III) are the same as those described for the substituent represented by Y₁, Y₂, Y₁, and Y₄ in General Formula (I), respectively.

Z₁, Z₂, Z₃, Z₄, and M respectively have the same meanings as those in General Formula (II), and the same applies to the description, specific examples, and preferred range thereof.

The phthalocyanine compound represented by the General Formula (I), (II), or (III) is still more preferably a phthalocyanine compound represented by the following General Formula (IV).

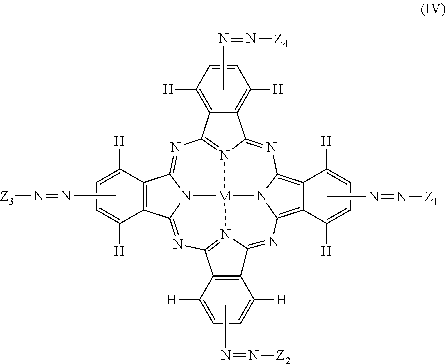

In the General Formula (IV),

Z₁, Z₂, Z₃, and Zs each independently represent a heterocyclic group, the heterocyclic group may have a substituent,

M represents a hydrogen atom, a metal element, a metal oxide, a metal hydroxide, or a metal halide, and

at least one of Zs, Z₂, Z₃, or Zs contains an ionic hydrophilic group.

Z₁, Z₂, Z₃, Z₄, and M in General Formula (IV) respectively have the same meanings as those in General Formula (II), and the same applies to the description, specific examples, and preferred range thereof.

The phthalocyanine compound represented by General Formula (I) contained in the dye ink composition according to the embodiment of the present invention may be one kind or two or more kinds.

Specific examples of the phthalocyanine compound represented by General Formula (I) include the following compounds, which are not limited thereto. “(t)C₄H9” and “C₄H9(t)” represent a t-butyl group.

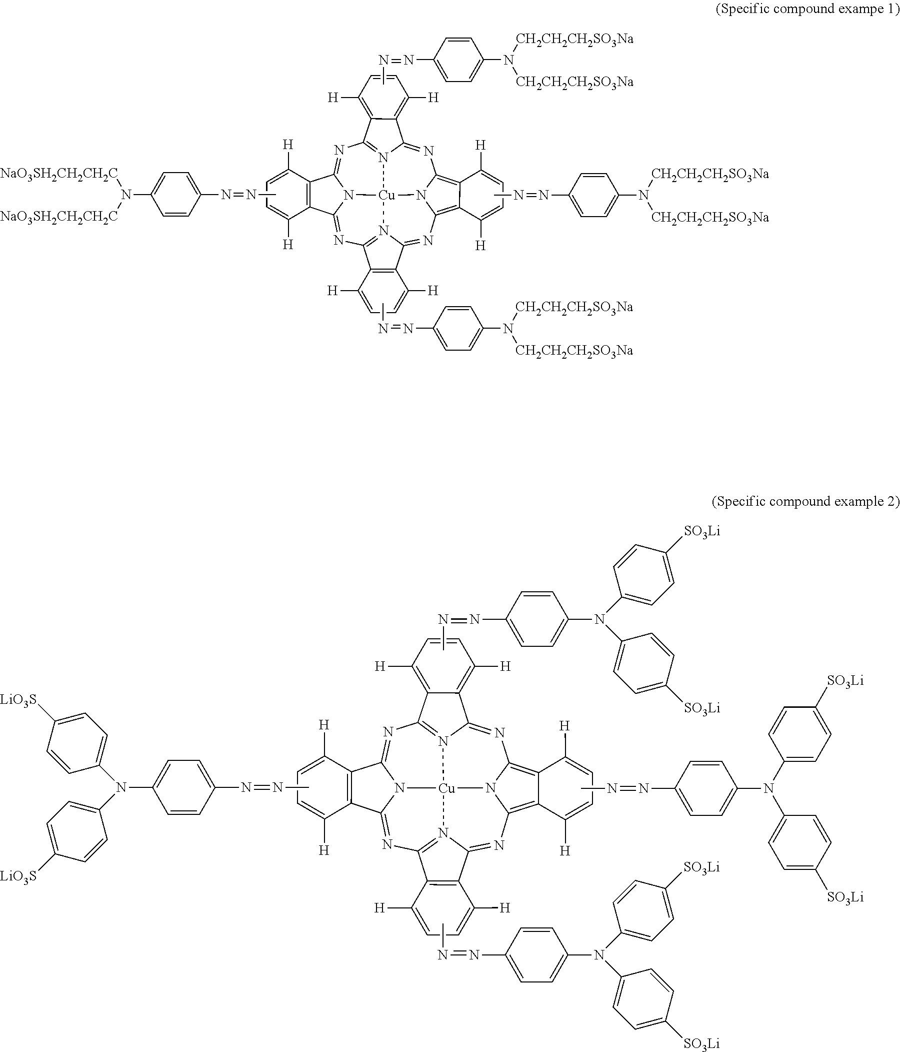

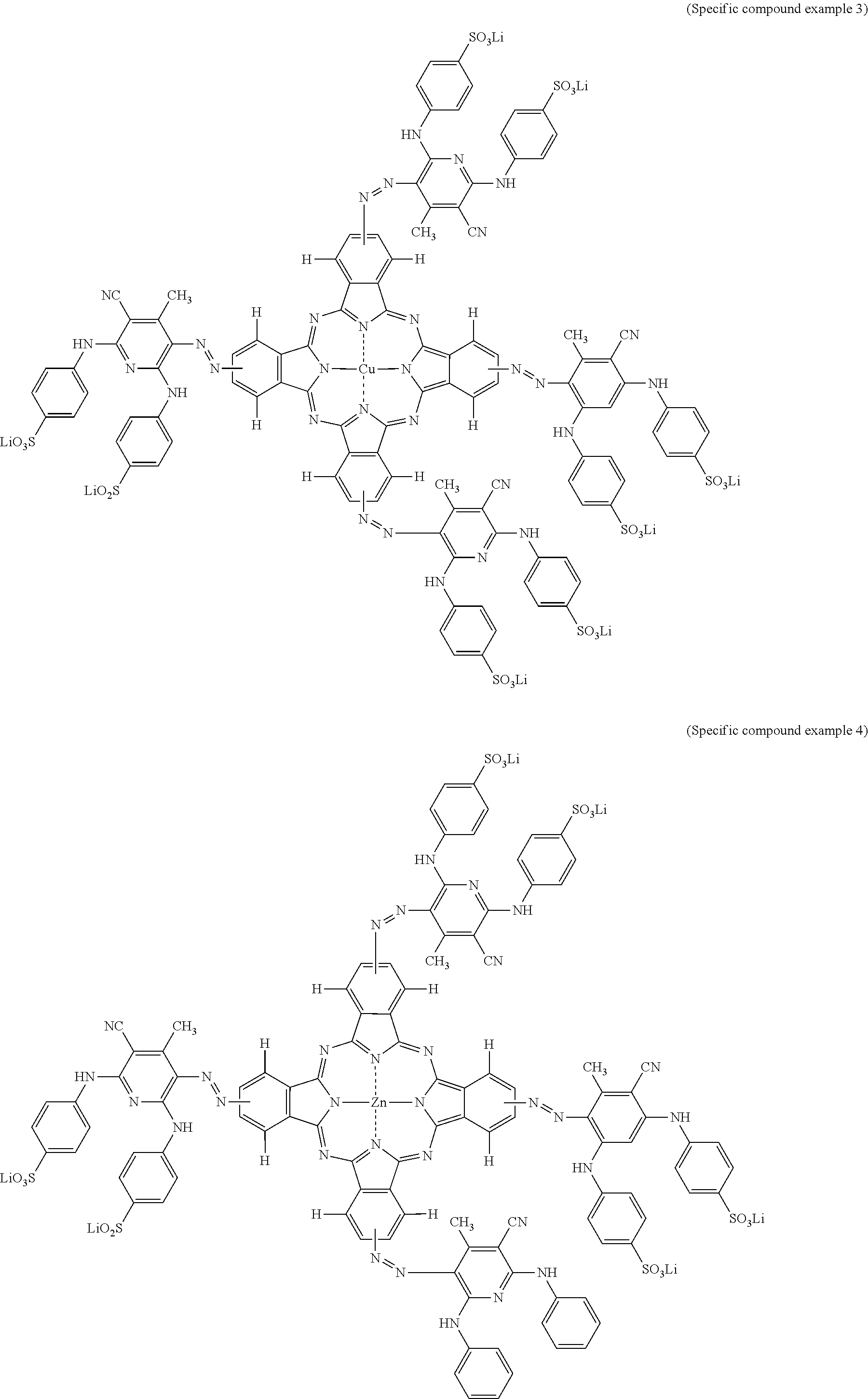

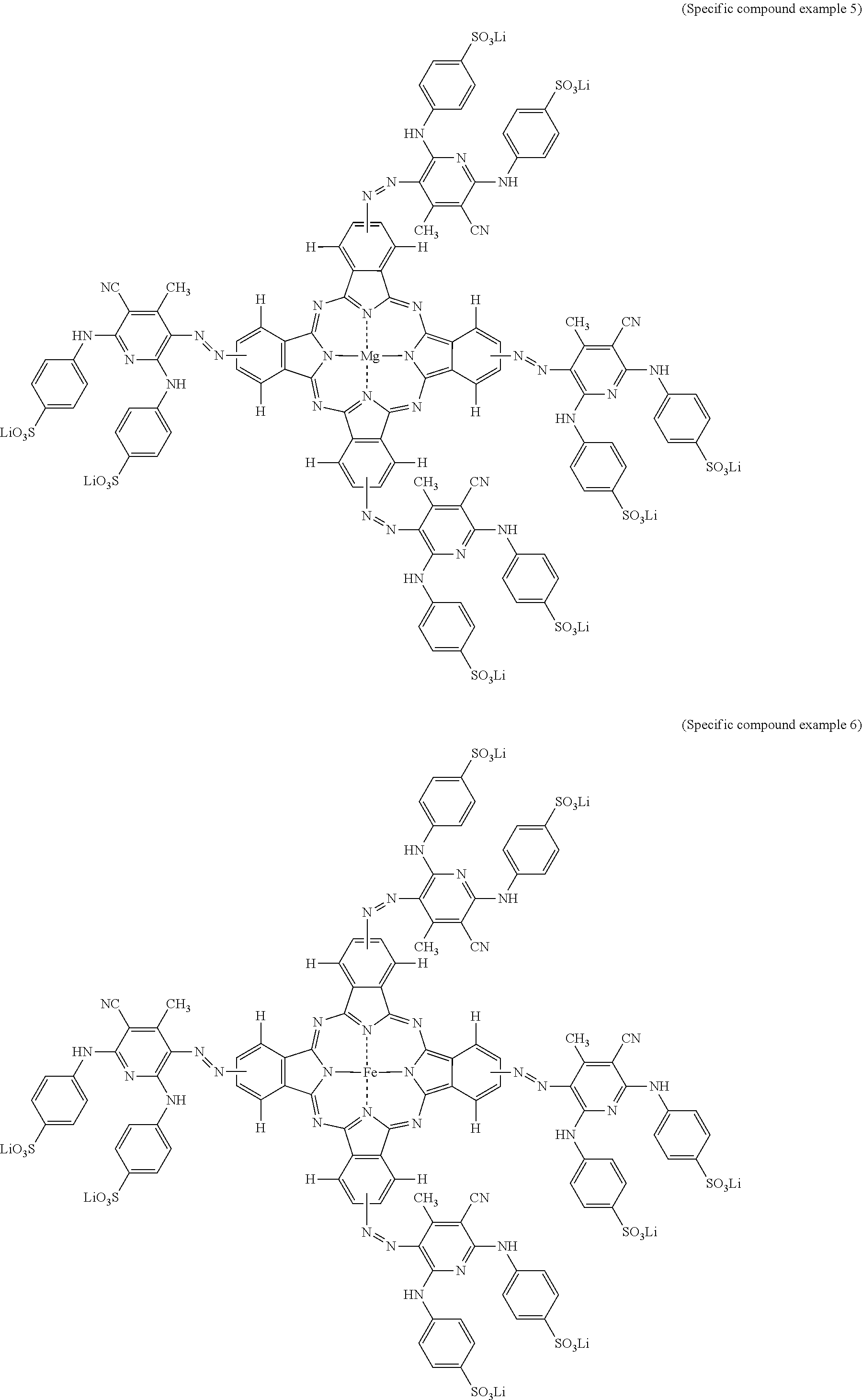

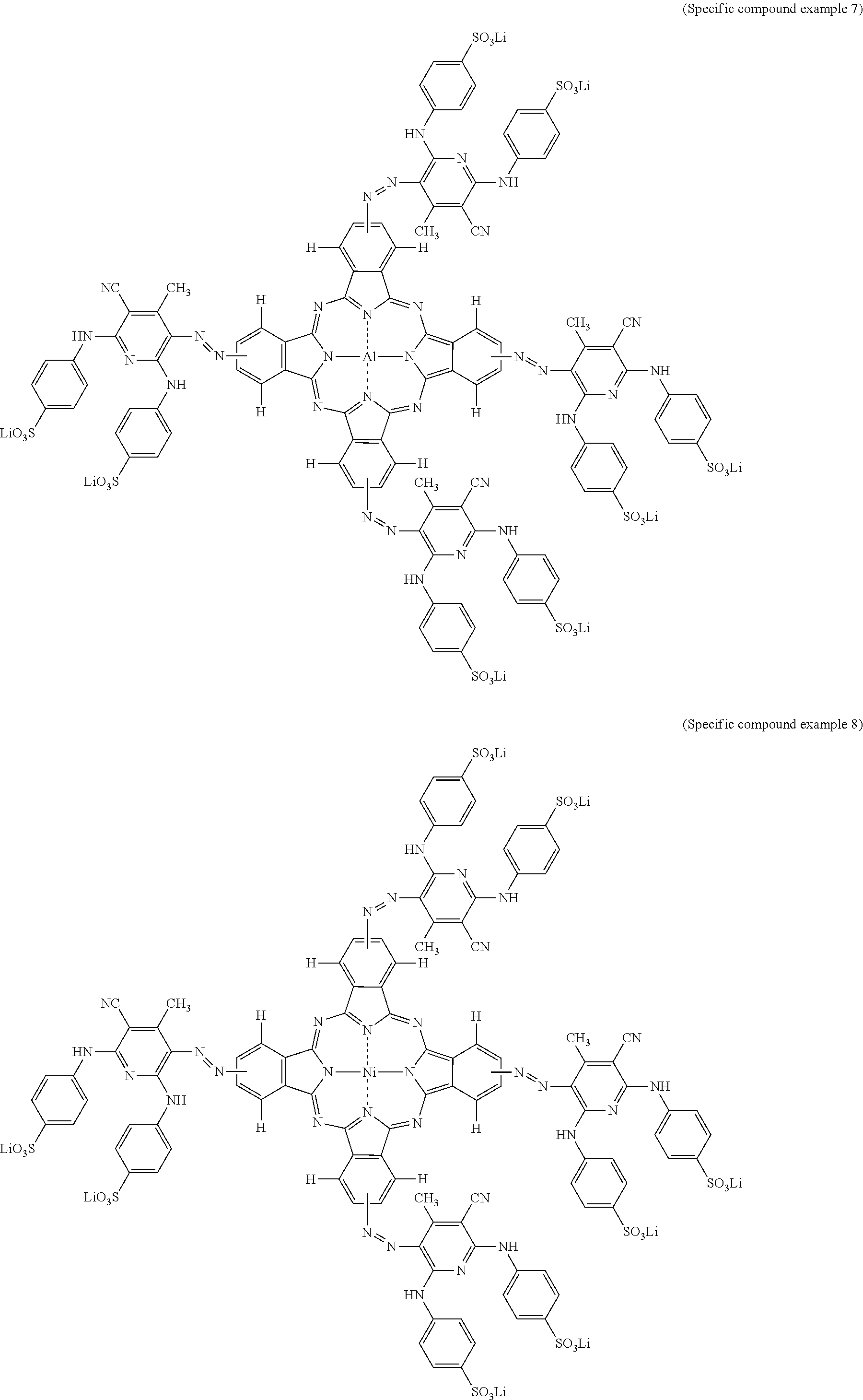

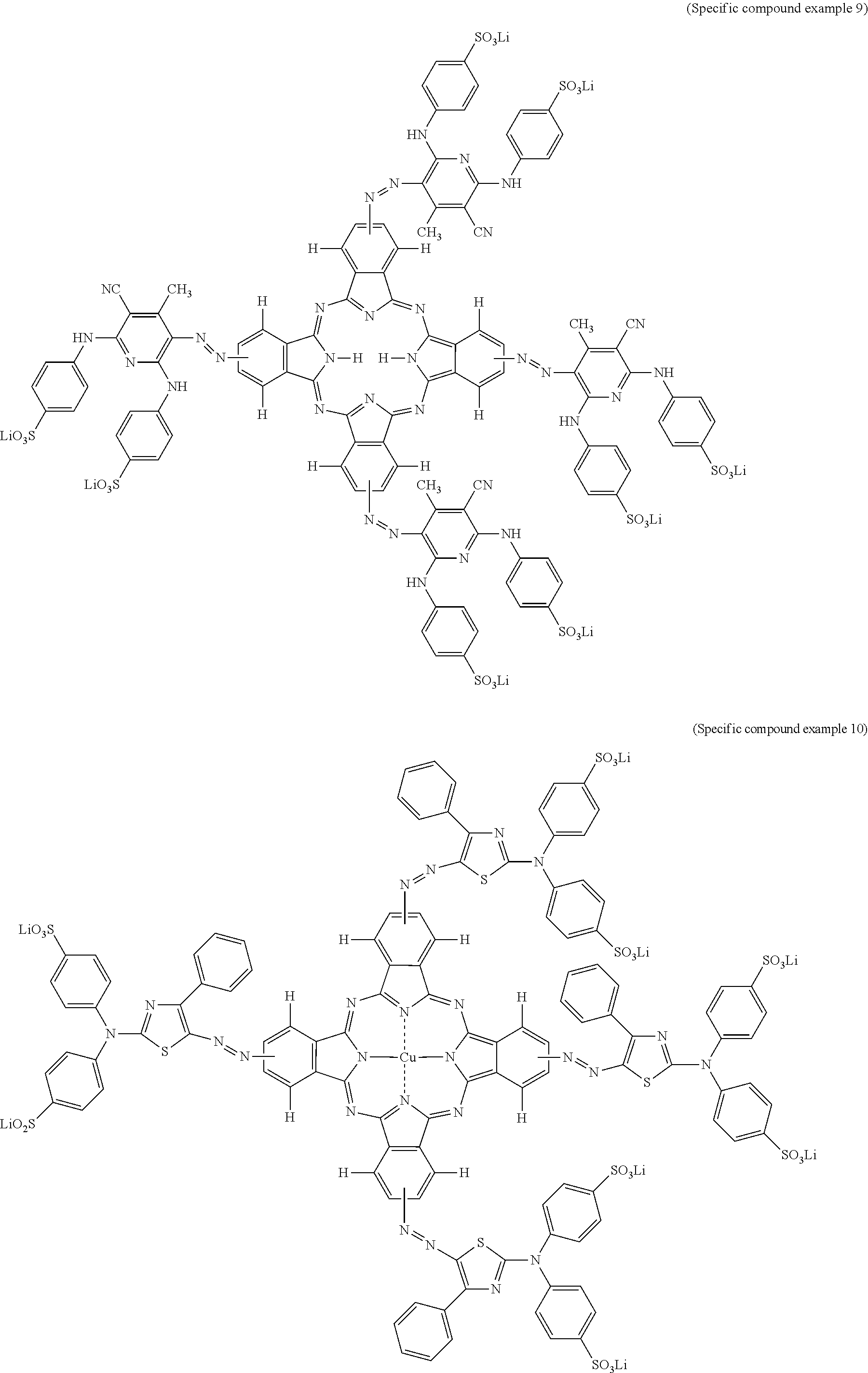

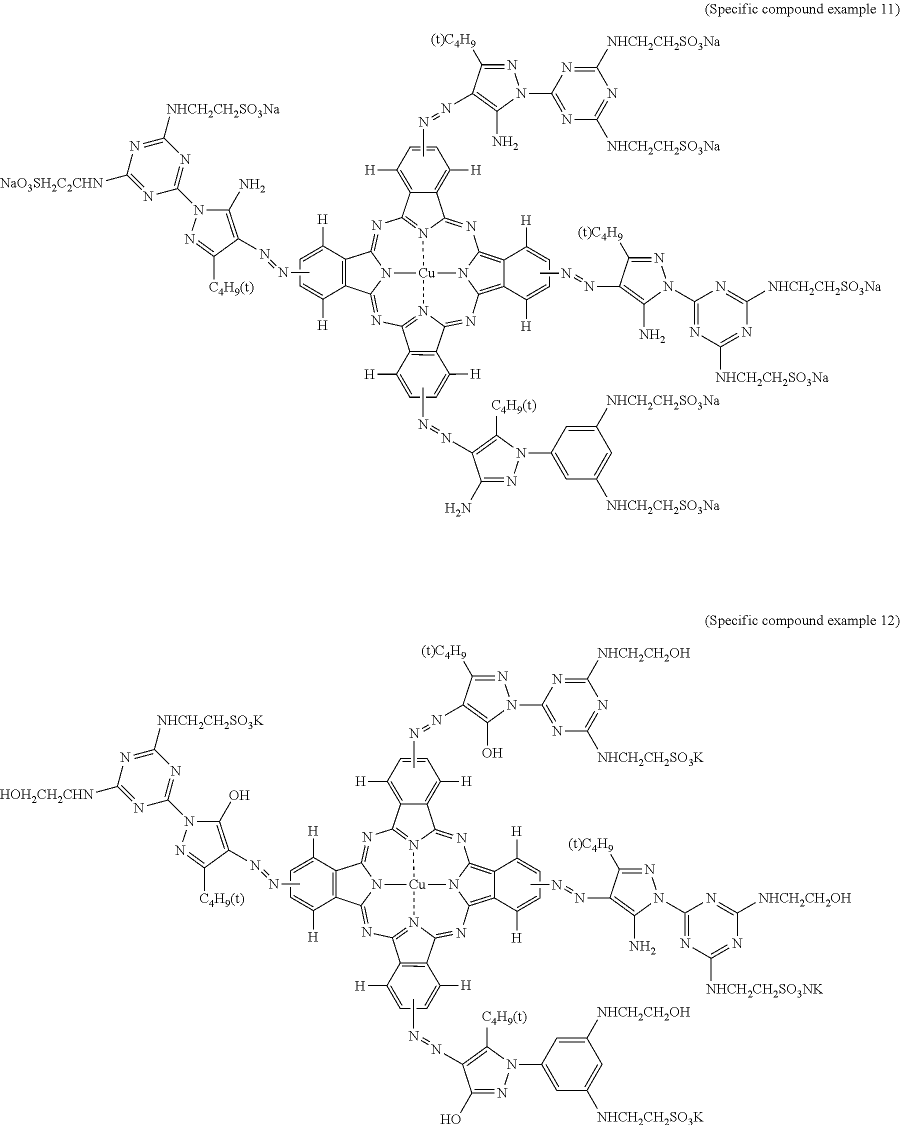

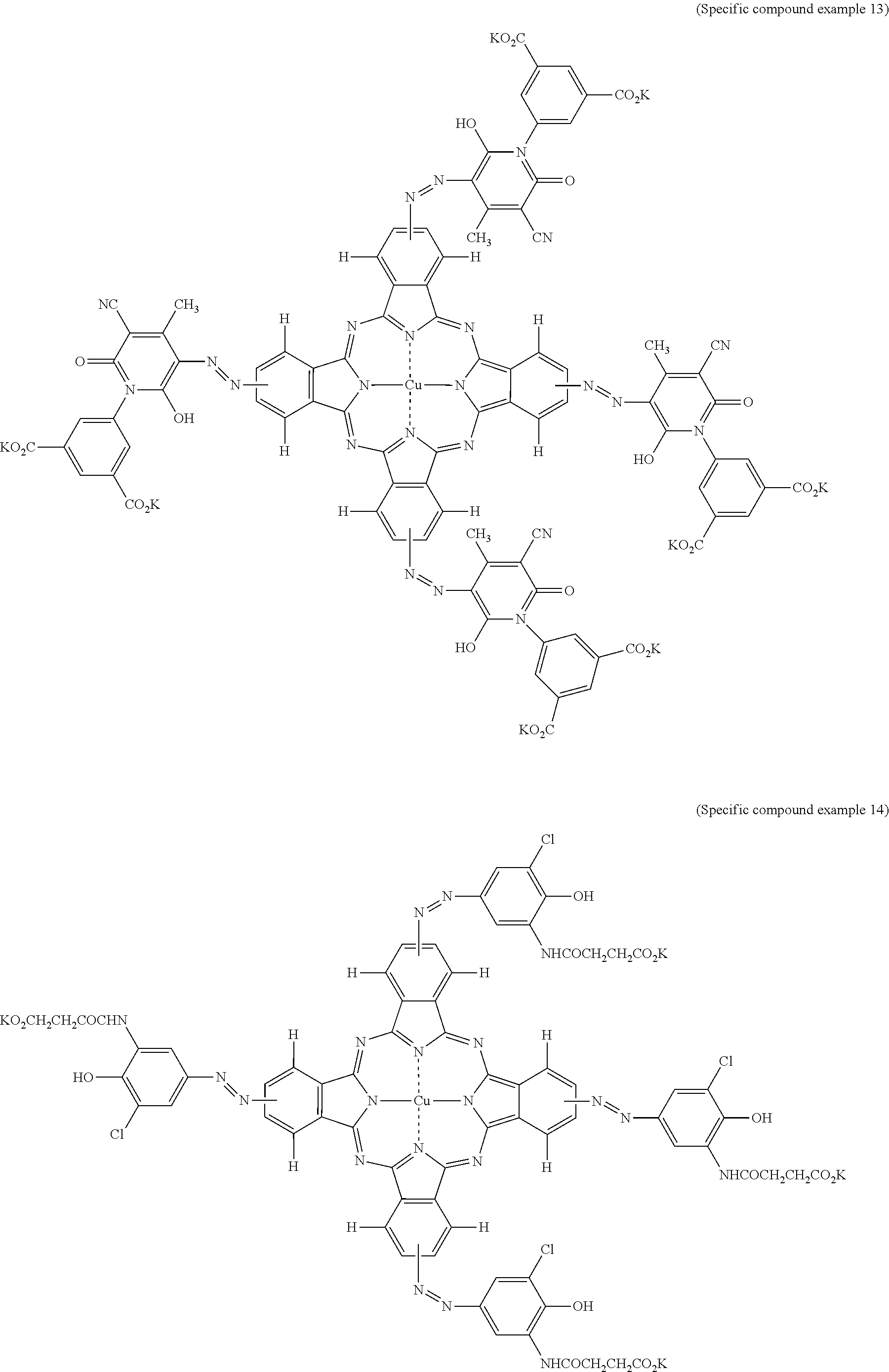

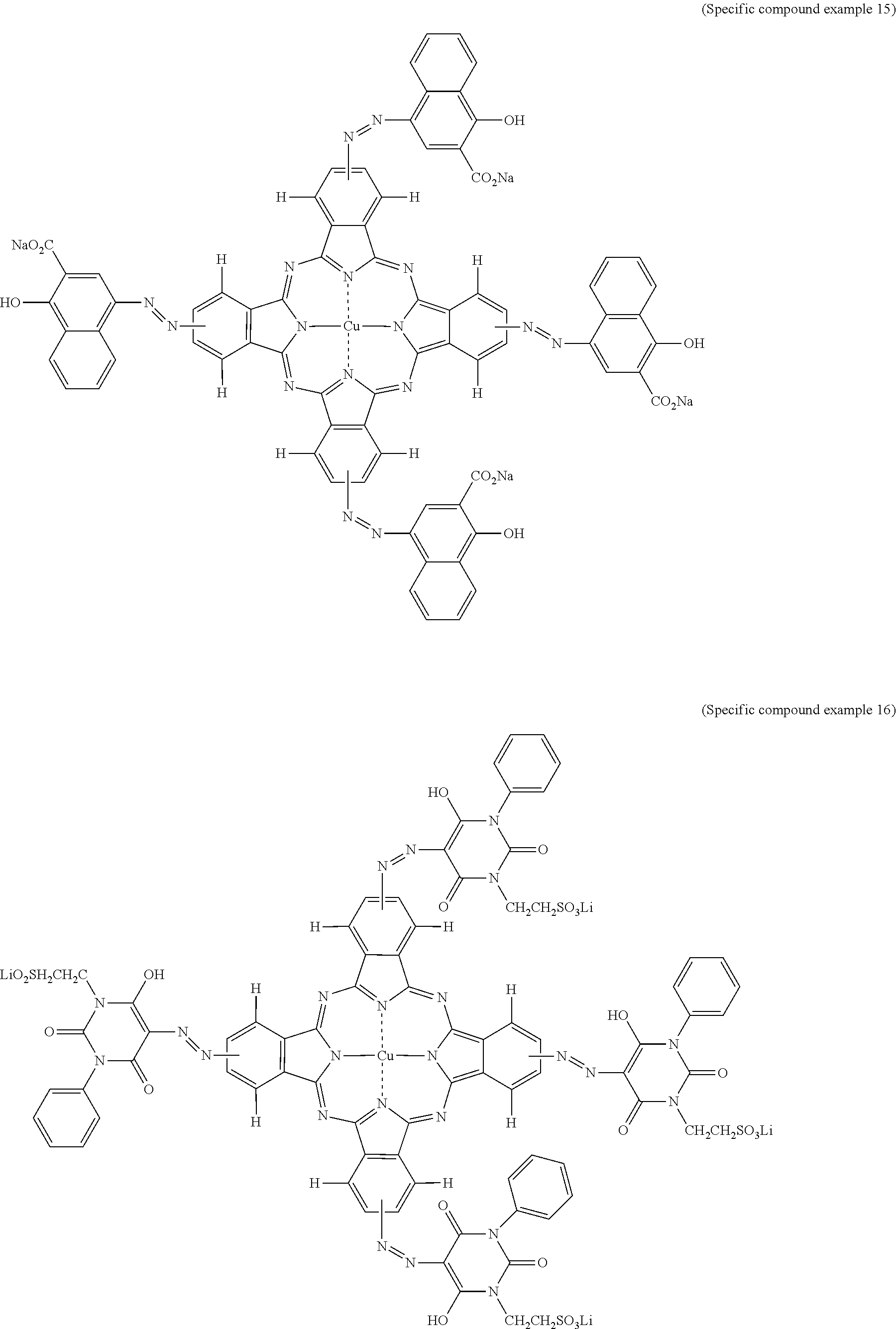

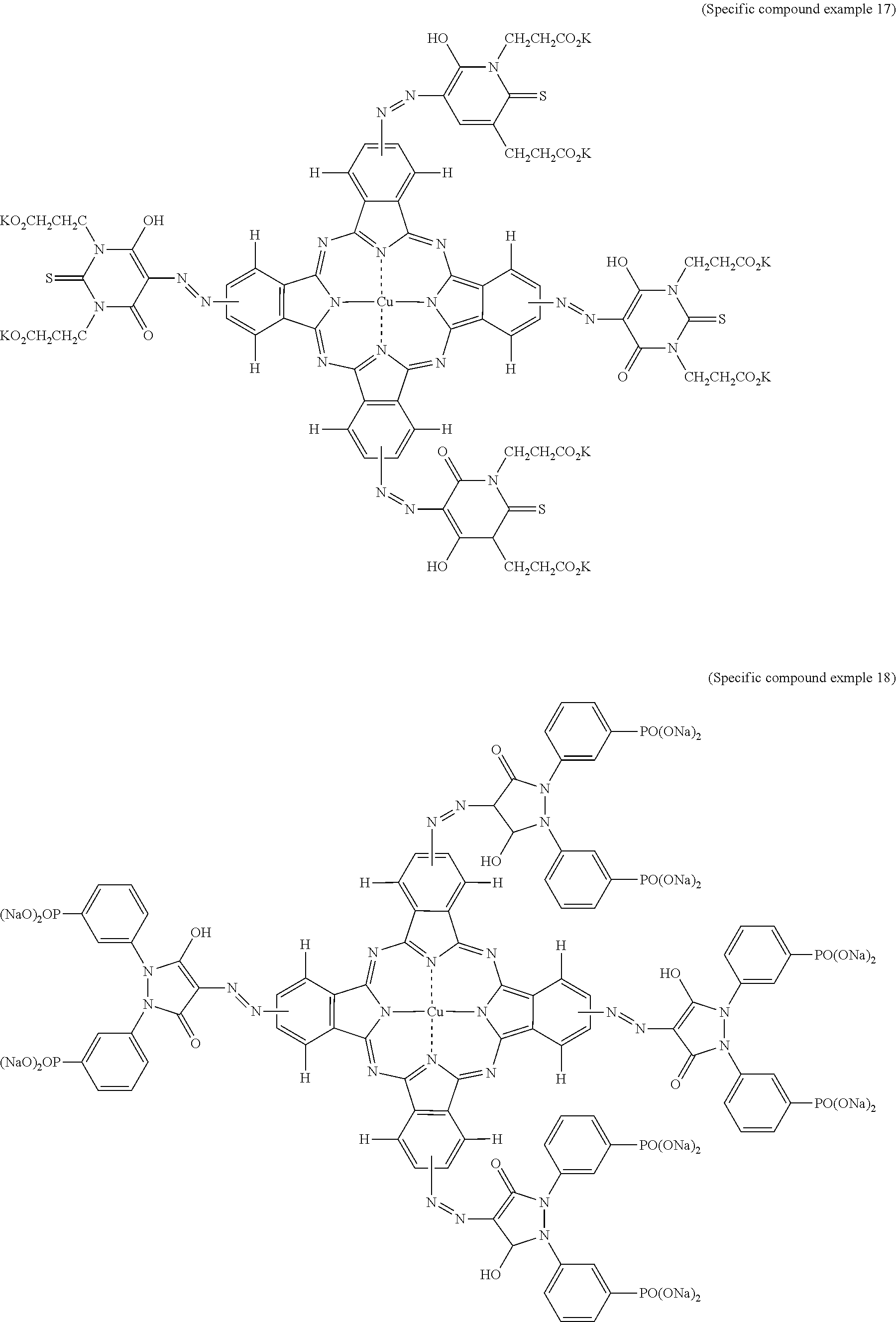

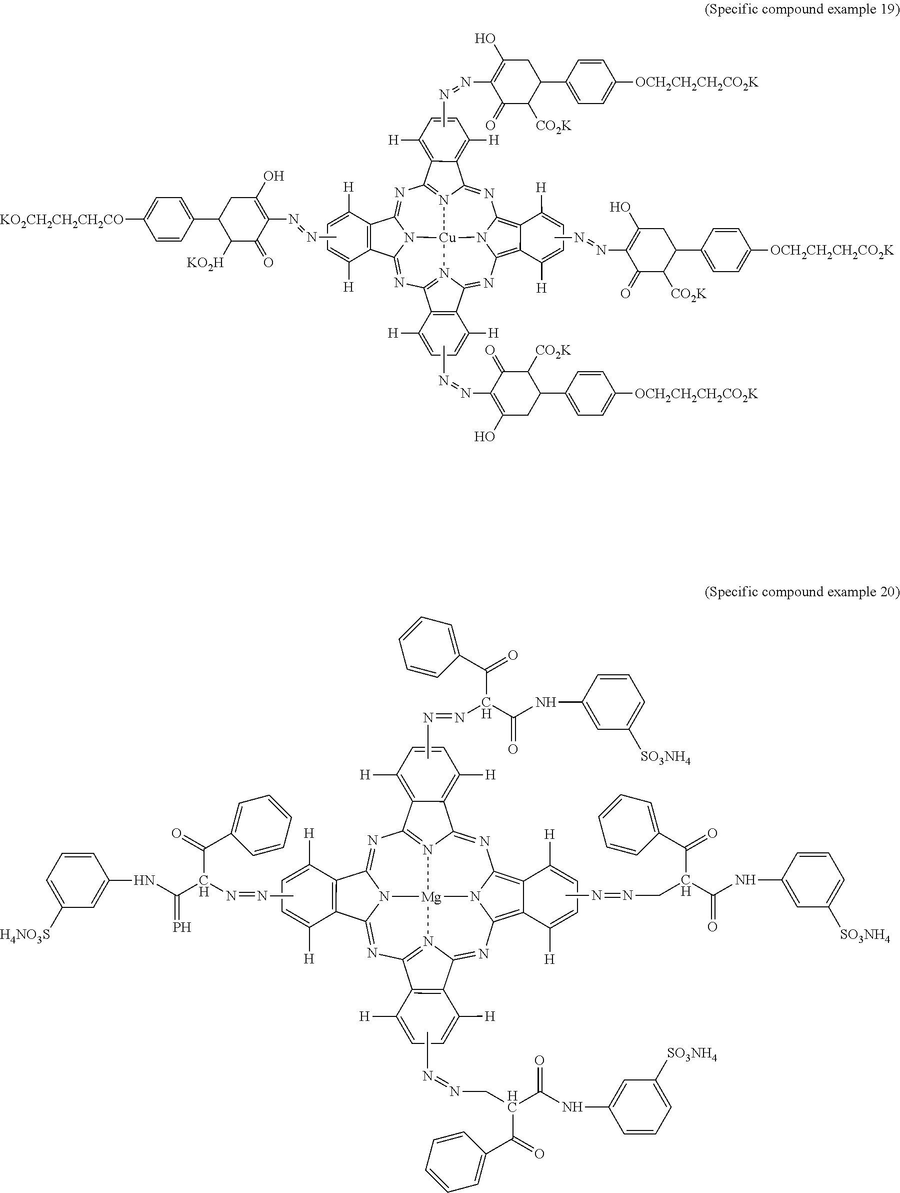

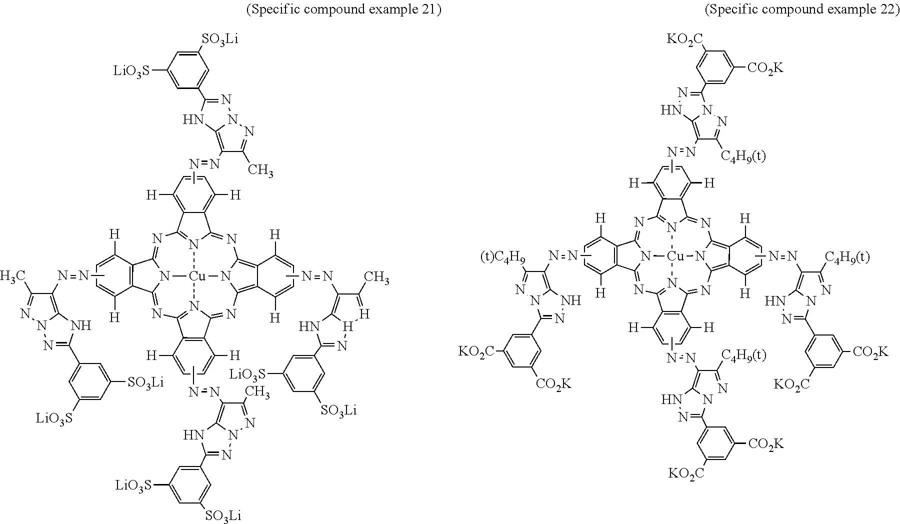

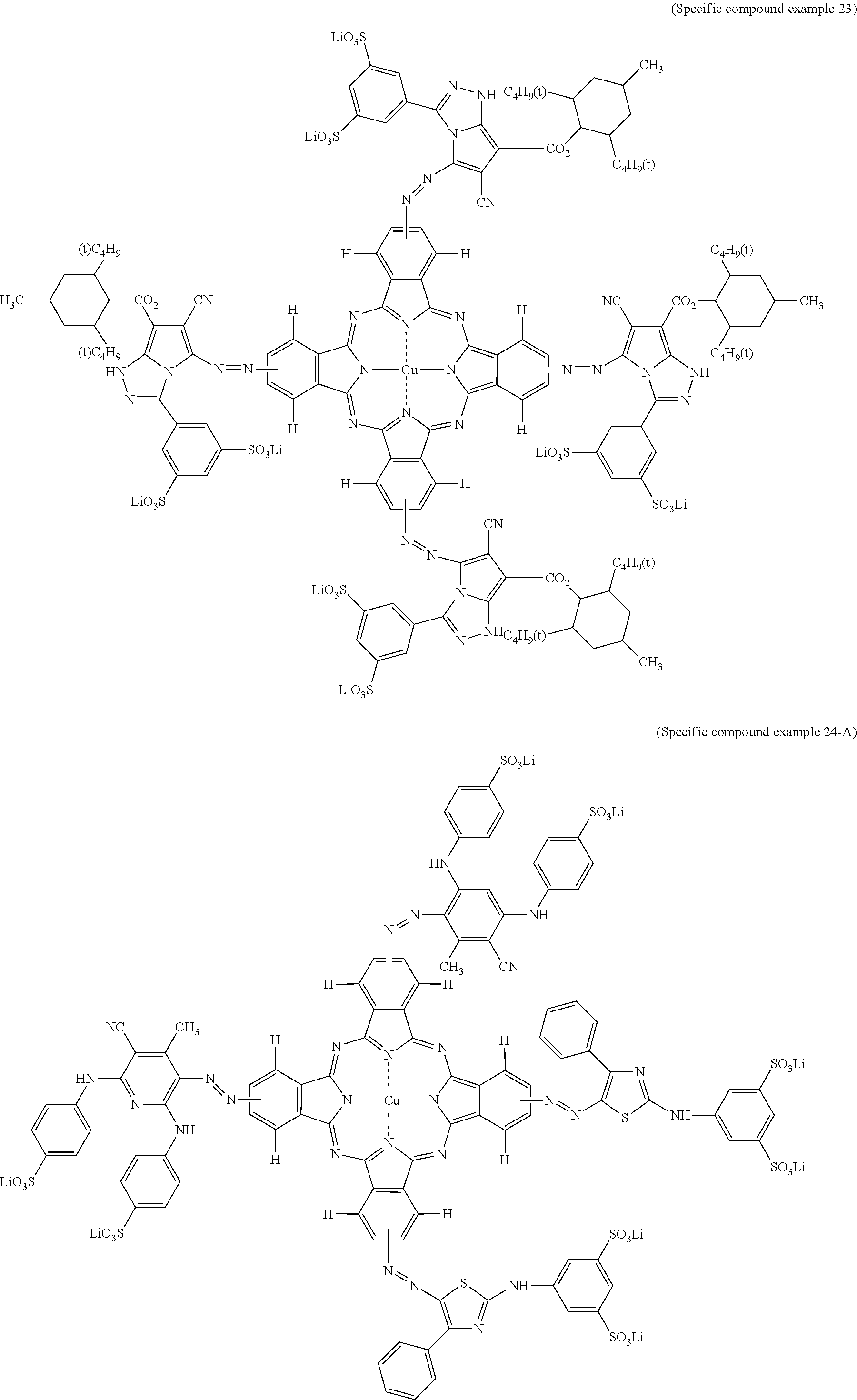

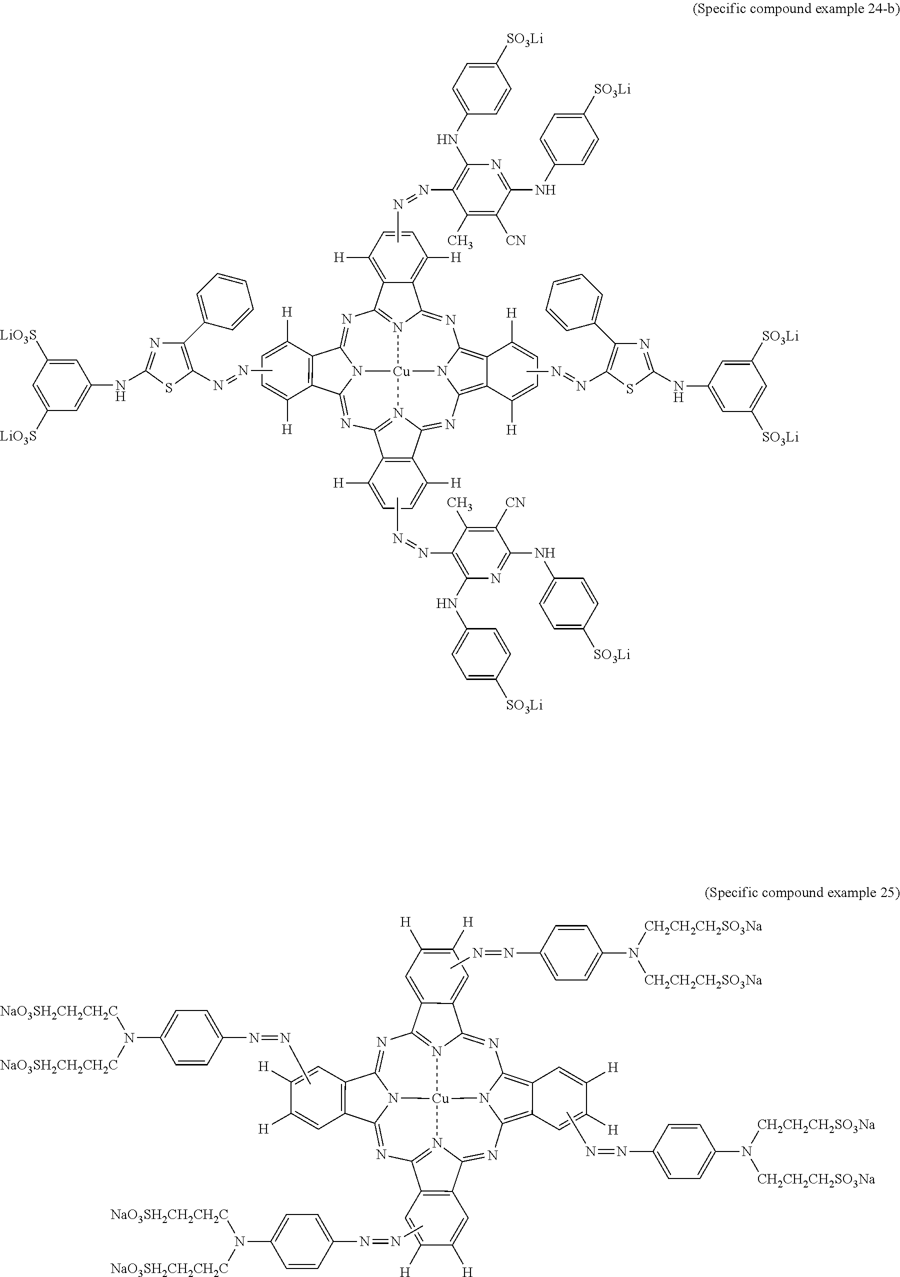

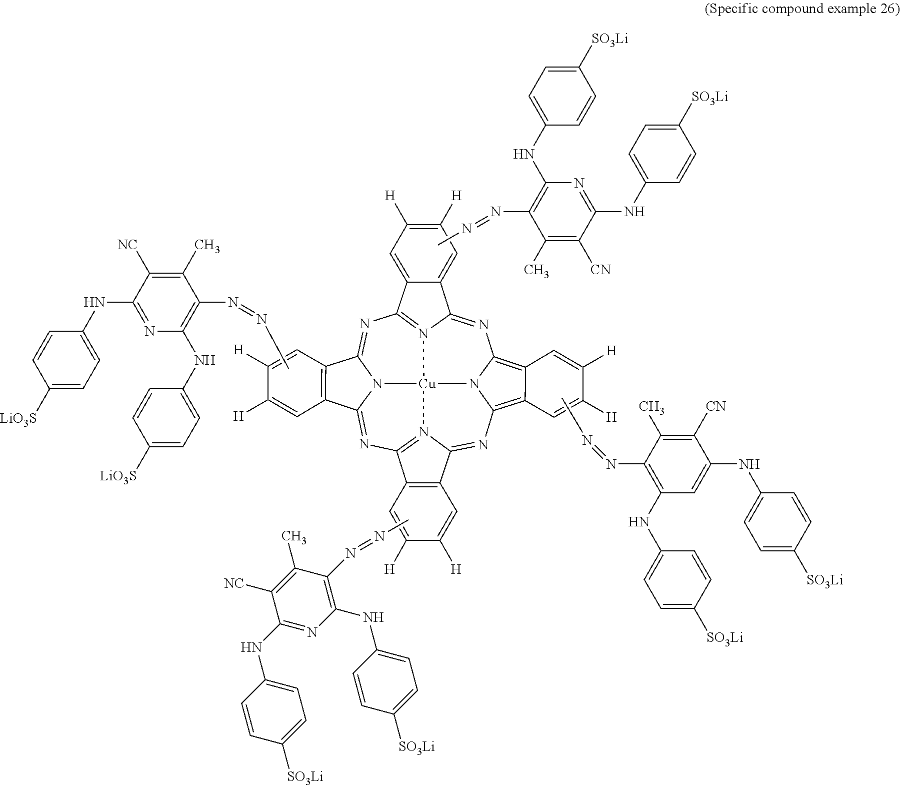

Singly or in a combination of two or more, by applying further purification as necessary, the phthalocyanine compound represented by General Formula (I) can be synthesized, isolated, and purified based on the known method (for example, the method described in “Phthalocyanine: Chemistry and Function” (P. 1 to 62), co-authored by Shirai and Kobayashi, published by 1PC ‘Phthalocyanines: Properties and Applications’ (P. 1 to 54), co-authored by C. C Leznoff and A. B. P. Lever, published by VCH, Russian Journal of General Chemistry, 2018, Vol. 88, No. 6, p. 1164-1171, Chemistry Letters Vol. 33, No. 11 (2004) p. 1450-1451, JP4512543B, JP4625644B, or JP4854250).

One example of the synthetic route of the phthalocyanine compound represented by General Formula (I) is shown below, which is not limited thereto.

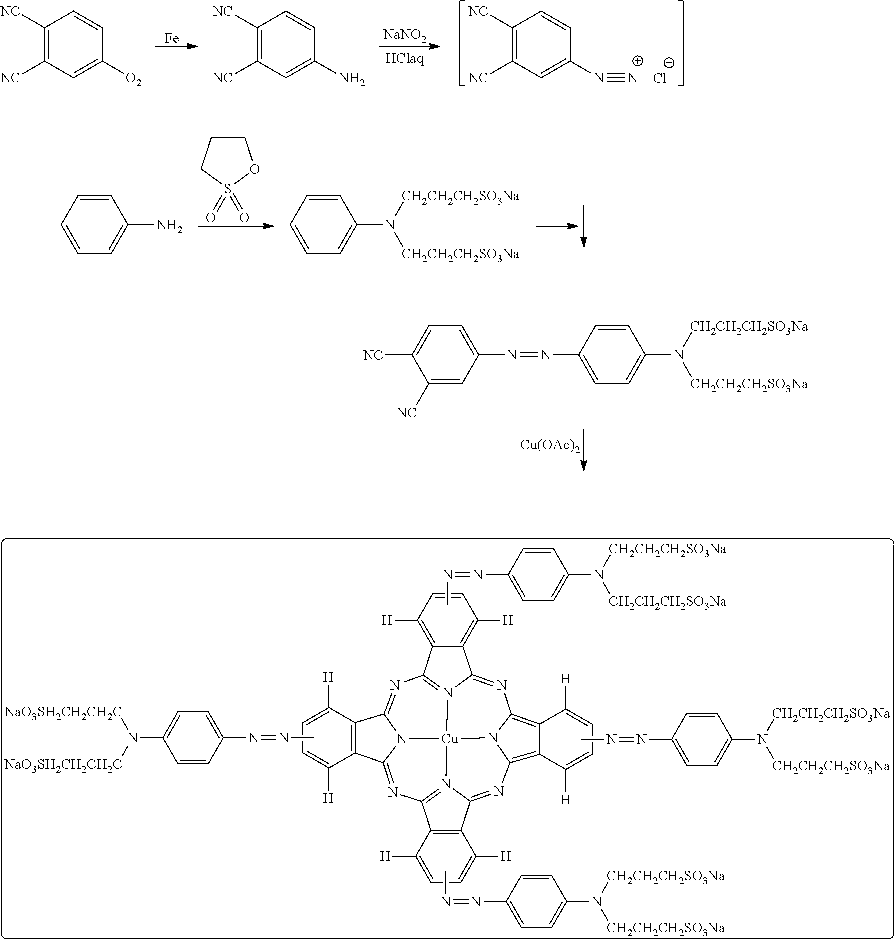

From the viewpoint of color tone, printing density, and storage stability, the content of the phthalocyanine compound represented by General Formula (I) with respect to the total mass of the dye ink composition according to the embodiment of the present invention is preferably 0.5% to 6.5% by mass, more preferably 1.0% to 6.5% by mass, and still more preferably 1.0% to 6.0°% o by mass in a case of being used as a dye ink in a range from gray to black.

The content of the phthalocyanine compound represented by General Formula (I) with respect to the total mass of the dye ink composition according to the embodiment of the present invention is preferably 1.0% to 2.0% by mass in a case of being used as a gray dye ink.

The content of the phthalocyanine compound represented by General Formula (I) with respect to the total mass of the dye ink composition according to the embodiment of the present invention is preferably 3.0% to 5.5% by mass in a case of being used as a black dye ink.

The dye ink composition according to the embodiment of the present invention may further contain other components in addition to the phthalocyanine compound represented by General Formula (I).

The dye ink composition according to the embodiment of the present invention can be used as a dye ink in a range from gray to black. Further, the dye ink composition according to the embodiment of the present invention can be used in the production of a dye ink in a range from gray to black.

The dye ink composition according to the embodiment of the present invention is preferably used as a black dye ink. Further, the dye ink composition according to the embodiment of the present invention is preferably used in the production of a black dye ink.

The black dye ink according to the embodiment of the present invention preferably has the above-described dye ink composition according to the embodiment of the present invention.

### Toning dye

In the dye ink composition according to the embodiment of the present invention, the phthalocyanine compound represented by General Formulae (1) to (IV) is preferably a coloring agent; however, a toning dye (a toning agent) may be used in combination in order to further improve the color tone in a range from gray to black.

Examples of the toning dye include A-1 to A-24, B-1 to B-18, C-1-1 to C-10-2, D-1 to D-10, E-1 to E-12, described in [0120] to [0146] of WO2021/039651A; (P-1) to (P-62-4) described in [0129] to [0172] of WO2020/262664A; (C-I-1) to (C-I-21) described in [0135] to [0142], and (C-II-1) to (C-II-24) described in [0183] to [0191] of WO2020/196435A; and Exemplary Compound I-1 to Exemplary Compound I-27 described in [0117] to [0127] of JP2020-76048A.

Further, specific examples of the toning dye include the compounds shown below.

In the following structural formulae, M represents a hydrogen atom or a counter cation, and it preferably represents a lithium ion, a sodium ion, a potassium ion, or an ammonium ion.

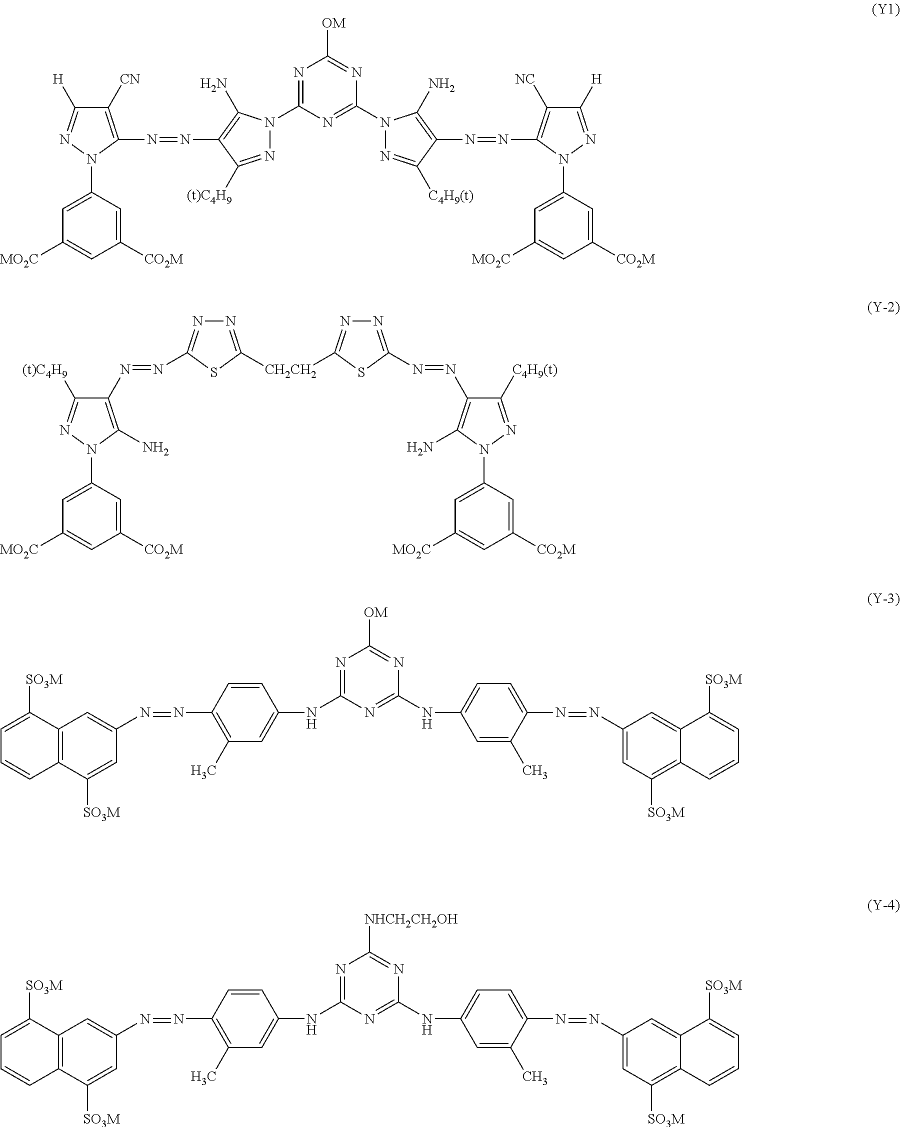

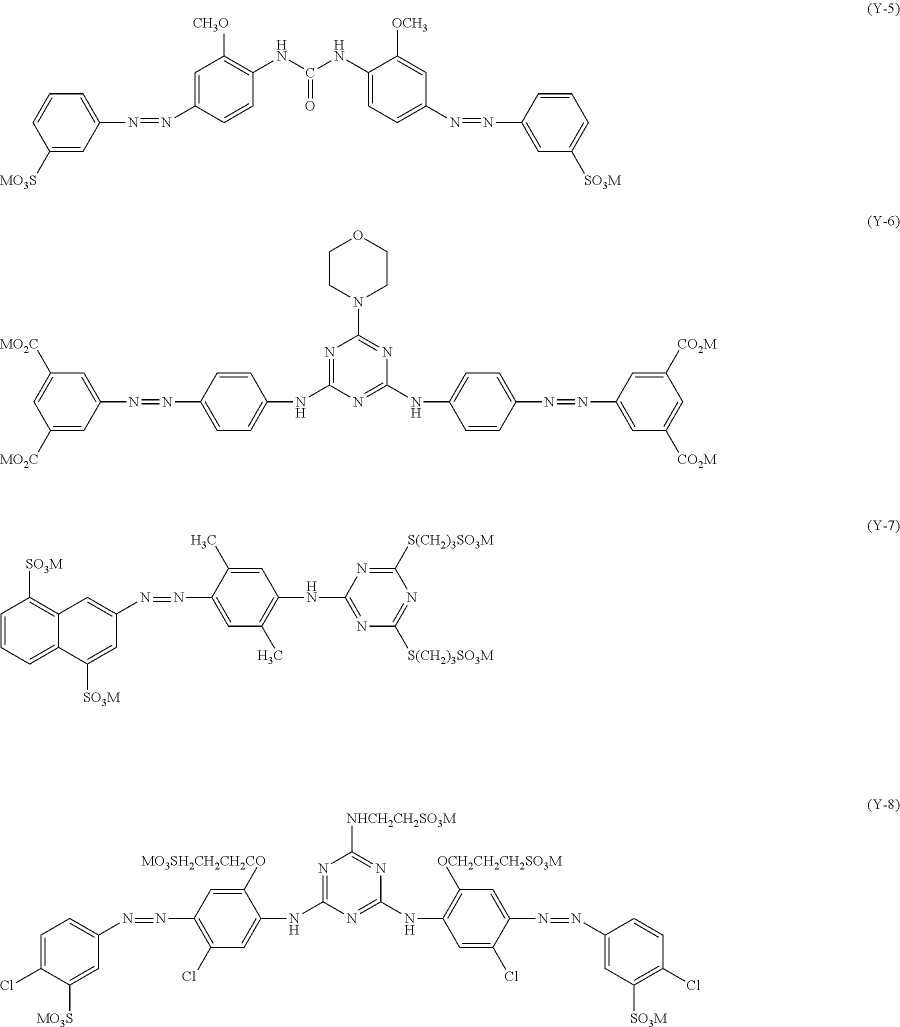

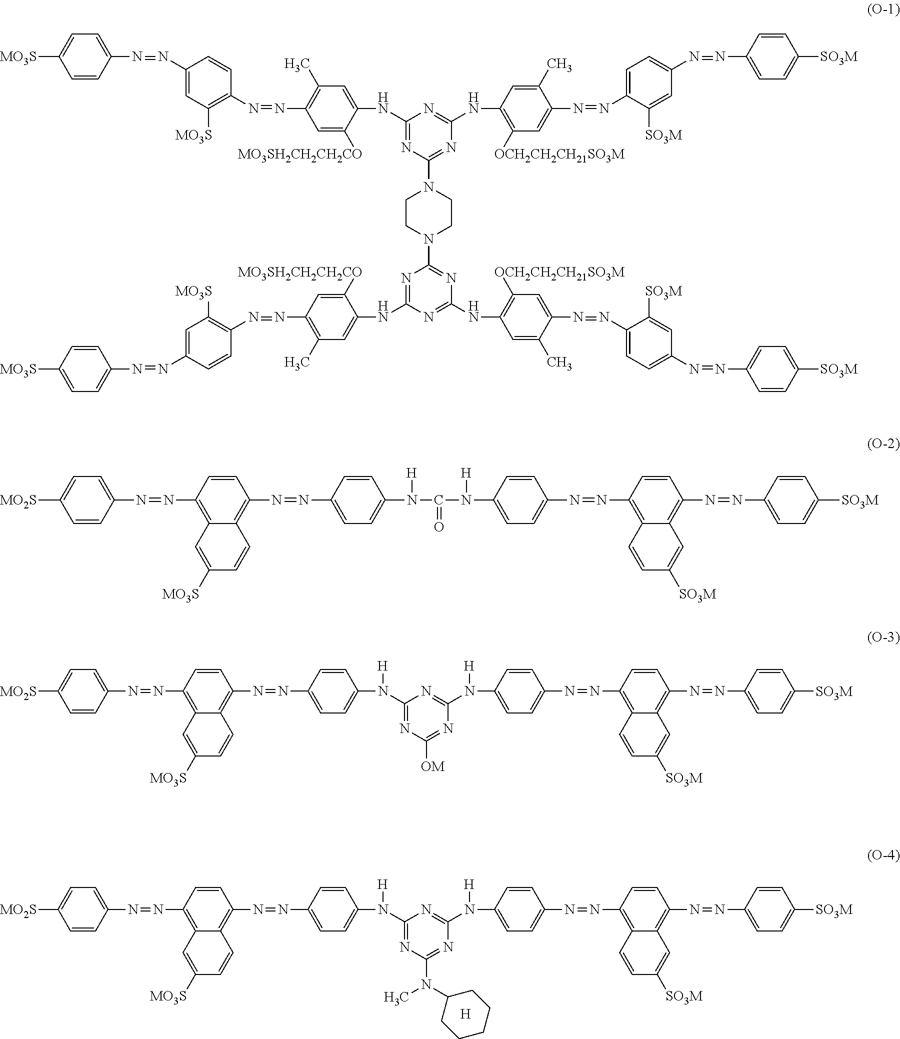

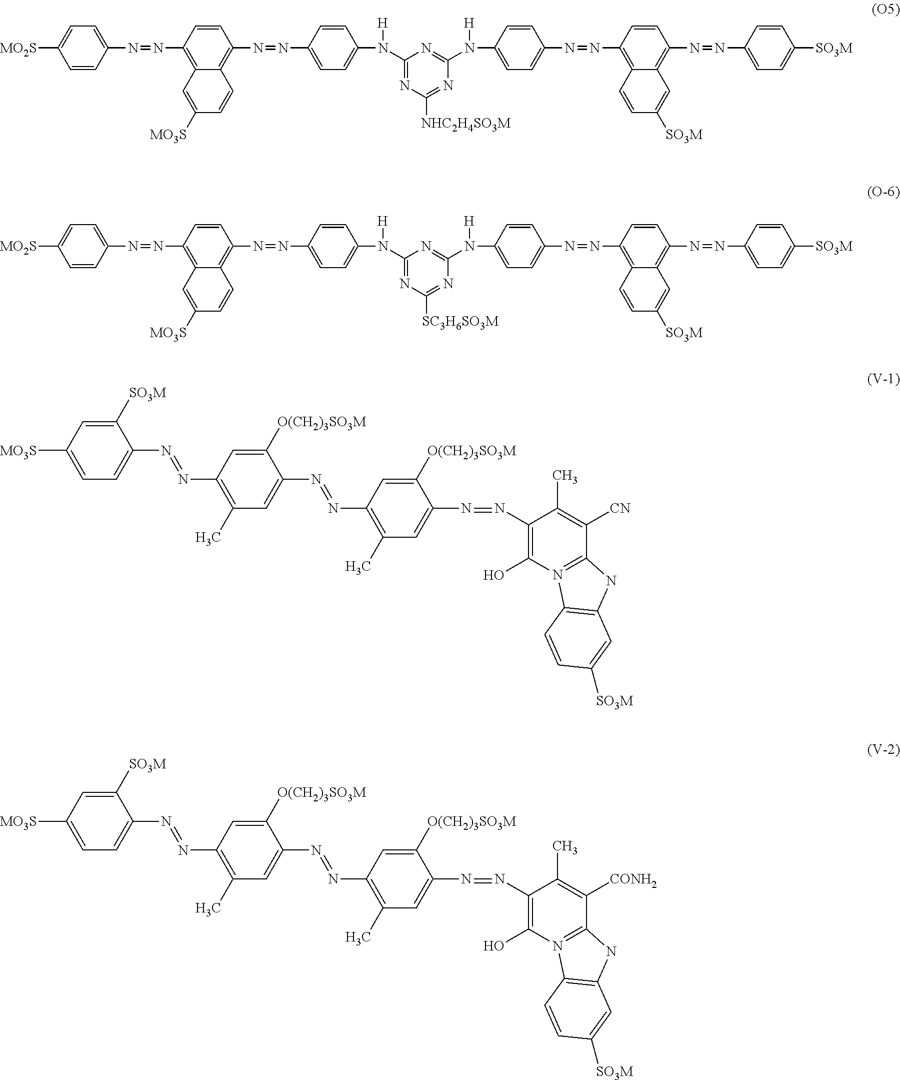

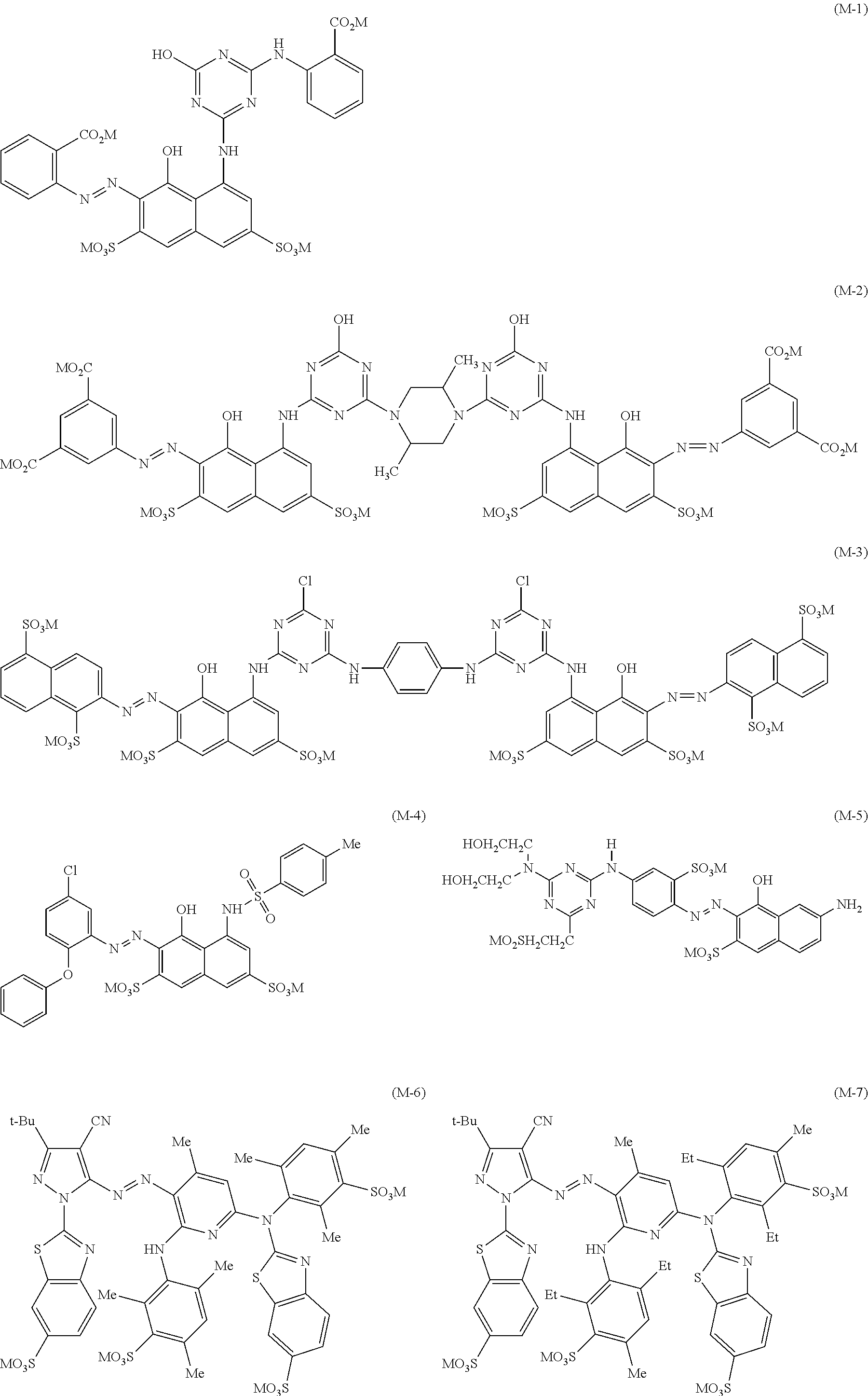

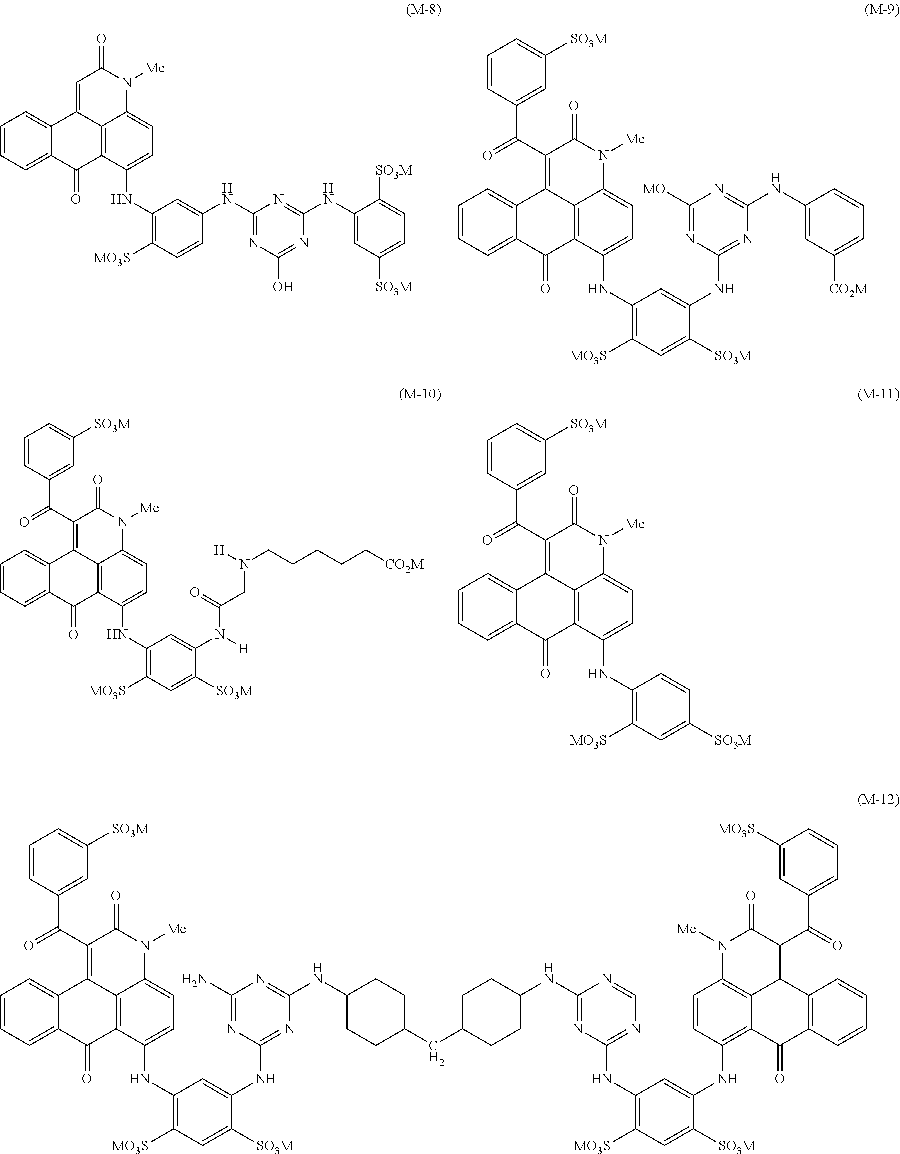

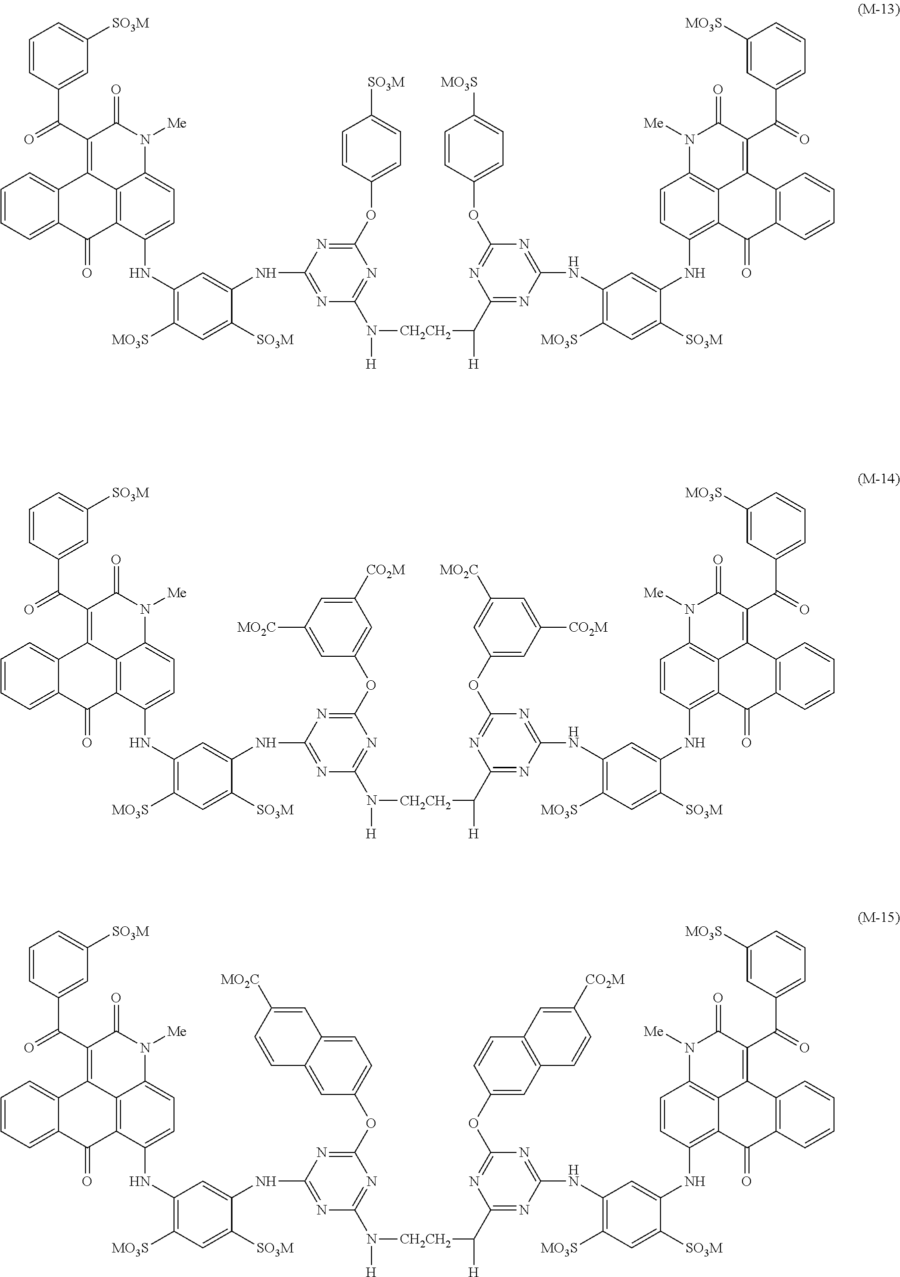

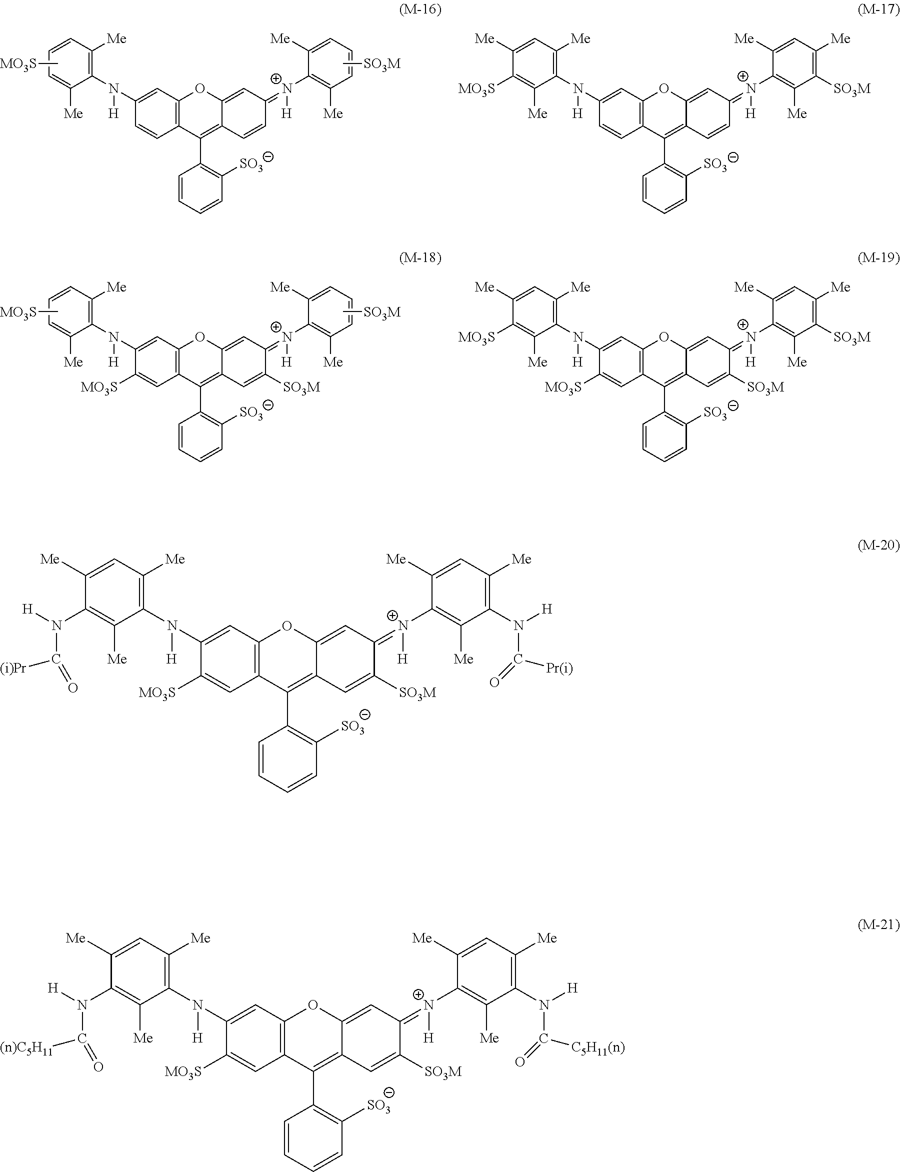

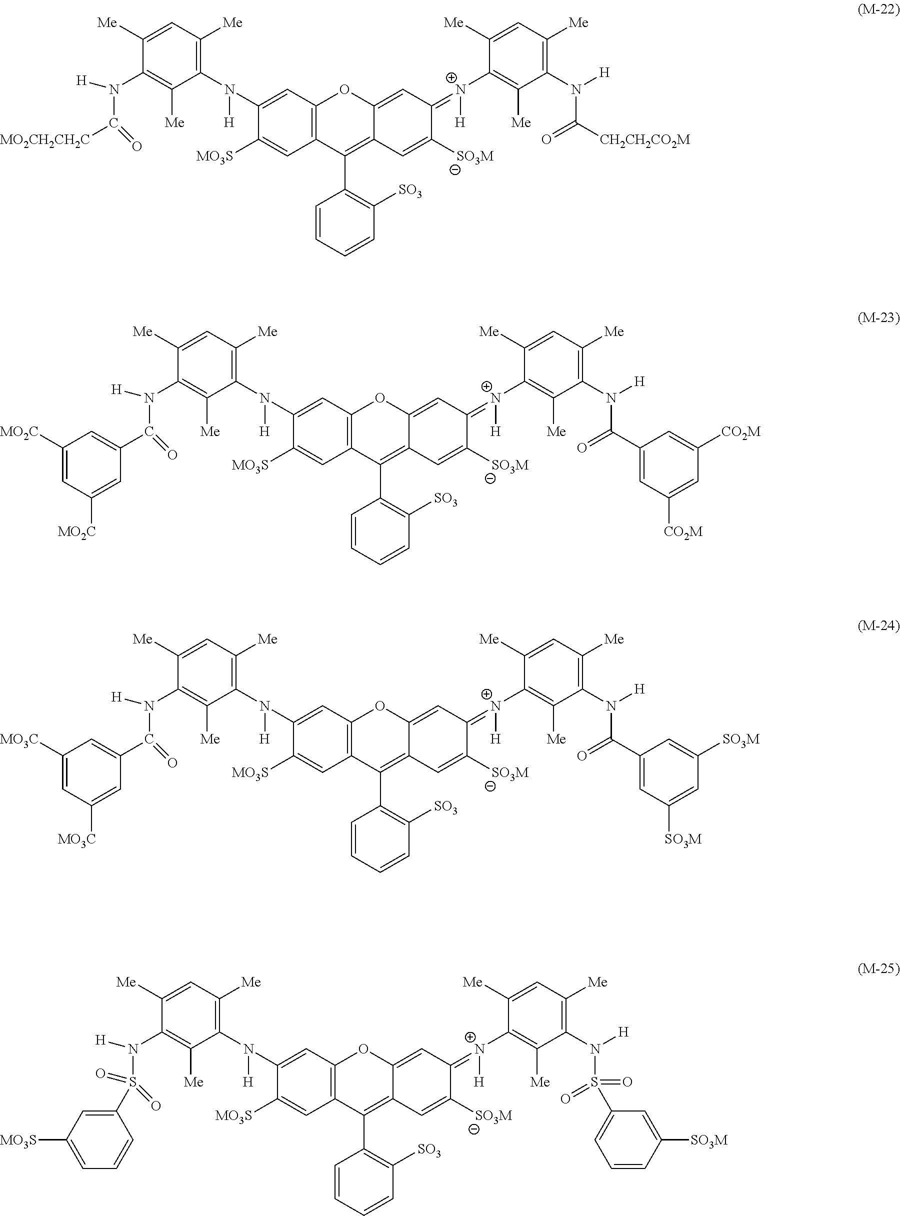

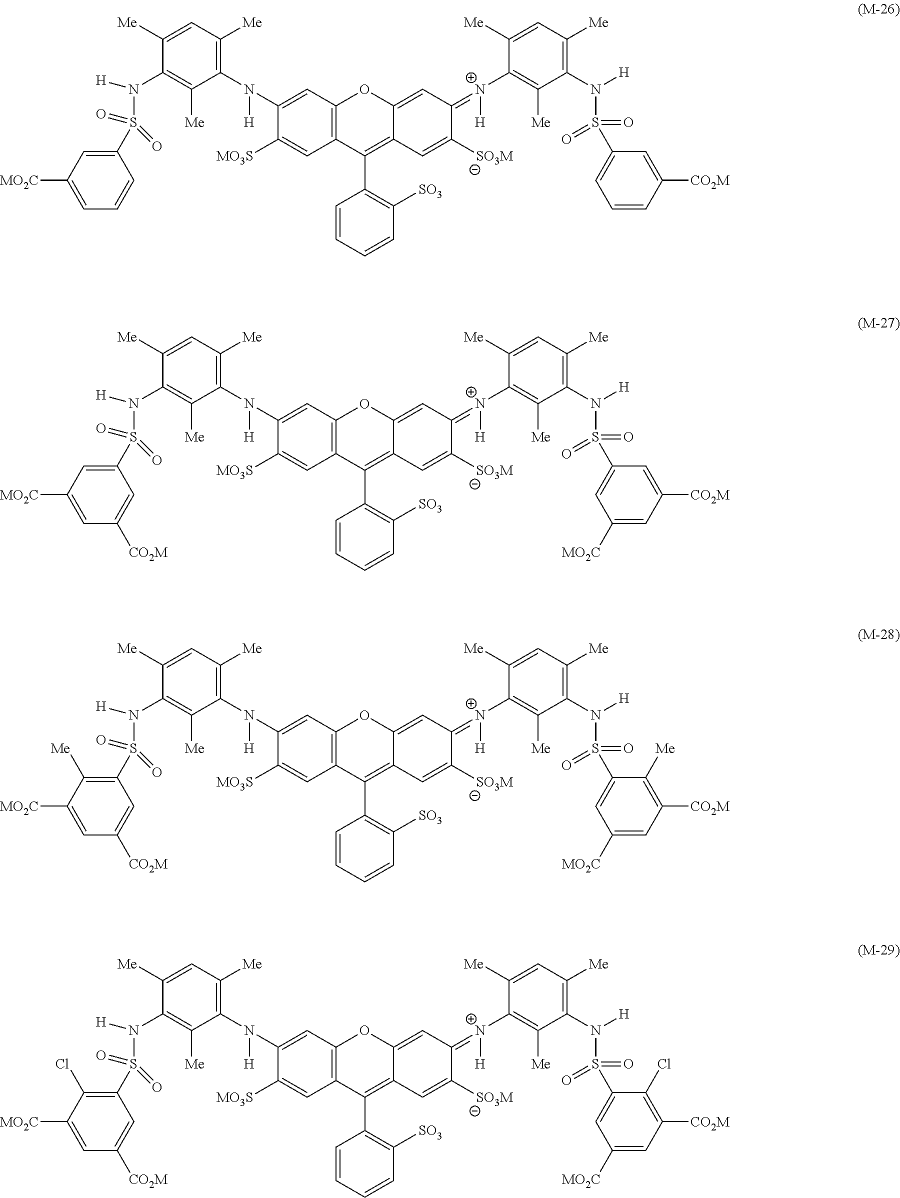

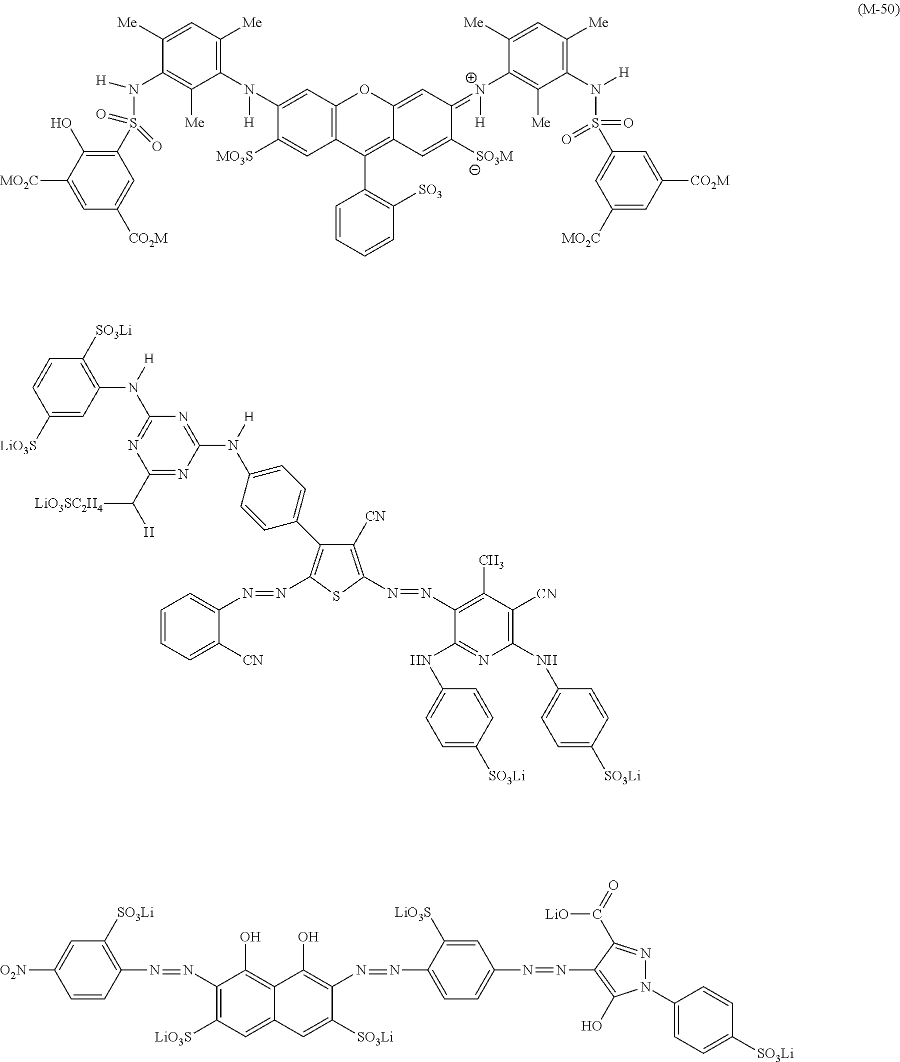

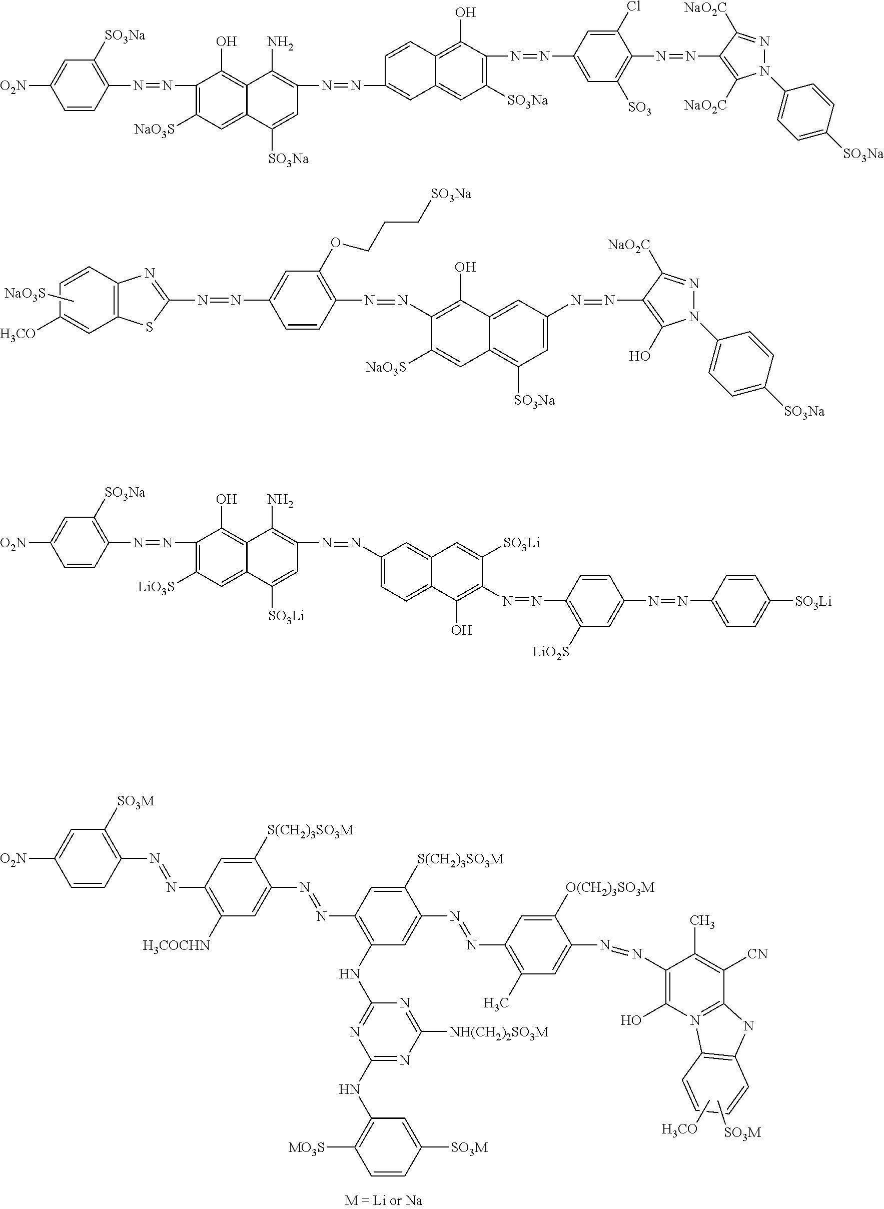

In the dye ink composition according to the embodiment of the present invention, in addition to the phthalocyanine compound represented by General Formula (I) and the toning dye that can be used in combination as necessary, other toning dyes can be further contained as a toning agent.

Examples of the representative dyes that can be used as a toning dye are shown below. It is noted that “C. I.” is an abbreviation for “color index”.

Examples of the yellow dye that is used in combination include C. I. Direct Yellow 8, 9, 11, 12, 27, 28, 29, 33, 35, 39, 41, 44, 50, 53, 59, 68, 86, 87, 93, 95, 96, 98, 100, 106, 108, 109, 110, 130, 132, 142, 144, 161, 163; C. I. Acid Yellow 17, 19, 23, 25, 39, 40, 42, 44, 49, 50, 61, 64, 76, 79, 110, 127, 135, 143, 151, 159, 169, 174, 190, 195, 196, 197, 199, 218, 219, 222, 227; C. I. Reactive Yellow 2, 3, 13, 14, 15, 17, 18, 23, 24, 25, 26, 27, 29, 35, 37, 41, 42; and C. I. Basic yellow 1, 2, 4, 11, 13, 14, 15, 19, 21, 23, 24, 25, 28, 29, 32, 36, 39, 40.

Examples of the magenta dye that is used in combination include C. 1. Direct Red 2, 4, 9, 23, 26, 31, 39, 62, 63, 72, 75, 76, 79, 80, 81, 83, 84, 89, 92, 95, 111, 173, 184, 207, 211, 212, 214, 218, 221, 223, 224, 225, 226, 227, 232, 233, 240, 241, 242, 243, 247; C. 1. Direct Violet 7, 9, 47, 48, 51, 66, 90, 93, 94, 95, 98, 100, 101; C. I. Acid Red 35, 42, 52, 57, 62, 80, 82, 111, 114, 118, 119, 127, 128, 131, 143, 151, 154, 158, 249, 254, 257, 261, 263, 266, 289, 299, 301, 305, 336, 337, 361, 396, 397; C. 1. Acid Violet 5, 34, 43, 47, 48, 90, 103, 126; C. I. Reactive Red 3, 13, 17, 19, 21, 22, 23, 24, 29, 35, 37, 40, 41, 43, 45, 49, 55; C. I. Reactive Violet 1, 3, 4, 5, 6, 7, 8, 9, 16, 17, 22, 23, 24, 26, 27, 33, 34; C. I. Basic Red 12, 13, 14, 15, 18, 22, 23, 24, 25, 27, 29, 35, 36, 38, 39, 45, 46; and C. I. Basic Violet 1, 2, 3, 7, 10, 15, 16, 20, 21, 25, 27, 28, 35, 37, 39, 40, 48.

Examples of the cyan dye include C. I. Direct Blue 1, 6, 10, 15, 22, 25, 55, 67, 68, 71, 76, 77, 78, 80, 84, 86, 87, 90, 98, 106, 108, 109, 151, 156 158, 159, 160, 168, 189, 192, 193, 194, 199, 200, 201, 202, 203, 207, 211, 213, 214, 218, 225, 229, 236, 237, 244, 248, 249, 251, 252, 264, 270, 276, 280, 282, 288, 291, 314; C. I. Acid Blue 9, 22, 25, 40, 41, 59, 62, 72, 76, 78, 80, 82, 92, 93, 102, 104, 106, 112, 113, 117, 120, 127:1, 129, 138, 143, 167, 175, 181, 185, 197, 205, 207, 220, 221, 224, 228, 229, 230, 232, 234, 247, 249, 254, 258, 260, 264, 271, 275, 277, 278, 279, 280, 283, 288, 310, 326, 357; C. I. Reactive Blue 2, 3, 5, 8, 10, 13, 14, 15, 17, 18, 19, 21, 25, 26, 27, 28, 29, 38; and C. I. Basic blue 1, 3, 5, 7, 9, 22, 26, 41, 45, 46, 47, 54, 57, 60, 62, 65, 66, 69, 71. However, they are not limited thereto.

In a case where the dye ink composition according to the embodiment of the present invention is used as a gray dye ink, the content of the toning dye is preferably 0.01% to 2.0% by mass, more preferably 0.01% to 1.5% by mass, still more preferably 0.02°/o to 1.3% by mass, particularly preferably 0.03% to 1.3% by mass, and most preferably 0.03% to 1.0% by mass, based on the total mass of the dye ink composition. The above range is preferable from the viewpoint of the color tone adjustment (neutral gray toning) with the coloring agent coexisting in the dye ink composition.

In a case where the dye ink composition according to the embodiment of the present invention is used as a black dye ink, the content of the toning dye is preferably 0.05% to 5.0% by mass, more preferably 0.1% to 3.5% by mass, still more preferably 0.3% to 3.0% by mass, particularly preferably 0.3% to 2.0% by mass, and most preferably 0.5% to 1.0% by mass, based on the total mass of the dye ink composition. The above range is preferable from the viewpoints of the color tone adjustment (black toning) with the coloring agent coexisting in the dye ink composition and the printing density.

Compound represented by General Formula (P)

The dye ink composition according to the embodiment of the present invention preferably contains a compound represented by the following General Formula (P). The compound represented by the General Formula (P) is typically used as an additive.

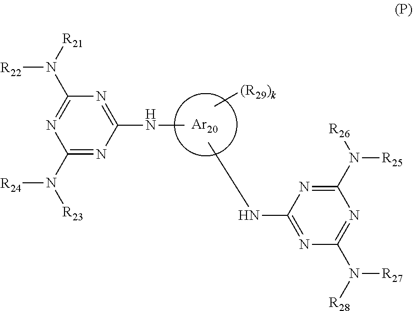

In the General Formula (P), Argo represents a benzene ring or a naphthalene ring; R₂₁ to Res each independently represent a hydrogen atom or a substituent; R₂i and R₂₂ may be bonded to each other to form a ring; R₂; and R₂₄ may be bonded to each other to form a ring; R₂₅ and R₂₆ may be bonded to each other to form a ring; R²⁷ and Res may be bonded to each other to form a ring; R₂₉ represents a substituent; in a case where Arlo represents a benzene ring, k represents an integer of 0 to 4; in a case where Arlo represents a naphthalene ring, k represents an integer of 0 to 6; in a case where a plurality of R₂₉'s are present, the plurality of R₂₉'s may be the same or different from each other; in a case where a plurality of R₂₉'s are present, the plurality of R₂₉'s may be bonded to each other to form a ring; and at least one of R₂₁ to R₂₉ has a hydrophilic group.

The compound represented by General Formula (P) is preferably a colorless water-soluble planar compound having more than 10 delocalized it electrons in one molecule.

A case where the number of it electrons that constitutes the delocalized n electron system increases and the n-electron system expands causes the absorption in the visible range in a large number of cases. In the present invention, “colorless” also includes a state in which an image is very slightly colored within a range that does not affect the image. The compound represented by General Formula (P) may be a fluorescent compound. However, it is preferably a compound having no fluorescence. It is more preferably a compound in which the wavelength (Amax) of the absorption peak on the longest wave side is 350 nm or less and more preferably 320 nm or less, and the molar absorption coefficient is 10,000 or less.

The upper limit of the number of delocalized it electrons in one molecule of the compound represented by General Formula (P) is not particularly limited; however, it is preferably 80 or less, more preferably, 50 or less, and particularly preferably 30 or less. Further, although more than 10 n electrons may form one large delocalized system, they may form two or more delocalized systems. In particular, a compound having three or more aromatic rings in one molecule is preferable. The aromatic ring may be an aromatic hydrocarbon ring, may be an aromatic heterocyclic ring containing a heteroatom, or may be fused to form one aromatic ring. Examples of the aromatic ring include a benzene ring, a naphthalene ring, an anthracene ring, a pyridine ring, a pyrimidine ring, a pyrazine ring, and a triazine ring.

The compound represented by General Formula (P) is preferably water-soluble, and it is preferably a compound, at least 1 g or more of which is dissolved in 100 g of water at 20° C. It is preferably a compound, 5 g or more of which is dissolved therein, and it is most preferably a compound, 10 g or more of which is dissolved therein.

In General Formula (P), R₂₁ to R₂₈ each independently represent a hydrogen atom or a substituent. Examples of the substituent include a halogen atom, a substituted or unsubstituted alkyl group, a substituted or unsubstituted alkenyl group, a substituted or unsubstituted alkynyl group, a substituted or unsubstituted aryl group, a substituted or unsubstituted heterocyclic group, a cyano group, a hydroxy group, a nitro group, a substituted or unsubstituted alkyloxy group,a substituted or unsubstituted aryloxy group,a substituted or unsubstituted heterocyclic oxy group,a substituted or unsubstituted alkylcarbonyl group,a substituted or unsubstituted alkylcarbonyloxy group,a substituted or unsubstituted alkyloxycarbonyl group,a substituted or unsubstituted arylcarbonyl group,a substituted or unsubstituted arylcarbonyloxy group,a substituted or unsubstituted aryloxycarbonyl group,a substituted or unsubstituted carbamoyl group,a substituted or unsubstituted carbamoyloxy group,a substituted or unsubstituted amino group,a substituted or unsubstituted mercapto group,a substituted or unsubstituted alkylthio group, a substituted or unsubstituted arylthio group, a substituted or unsubstituted heterocyclic thio group,a substituted or unsubstituted sulfamoyl group, a substituted or unsubstituted alkylsulfinyl group, a substituted or unsubstituted arylsulfinyl group, a substituted or unsubstituted alkylsulfonyl group,a substituted or unsubstituted arylsulfonyl group, a substituted or unsubstituted phosphino group, a substituted or unsubstituted phosphinyl group, a substituted or unsubstituted silyl group, a substituted or unsubstituted silyloxy group, and an ionic hydrophilic group. In a case where these substituents can further have one or more substituents, examples of this substituent that can be further contained also include a group having a substituent selected from the above-described substituents.

It is preferable that R₂₁ to R₂₈ each independently represent a hydrogen atom or a substituted or unsubstituted alkyl group. The alkyl group is preferably an alkyl group having 1 to 12 carbon atoms, more preferably an alkyl group having 1 to 8 carbon atoms, and most preferably an alkyl group having 1 to 6 carbon atoms. It is preferable that the alkyl group has a hydrophilic group described later as a substituent from the viewpoint of storage stability of the dye ink composition.

Each pair of R₂₁ and R₂₂, R₂; and R₂₄, R₂₅ and R₂6, and R₂₇ and R₂₈ may be bonded to form a ring. The ring is not particularly limited; however, it may be an aromatic ring or a non-aromatic ring, and it is preferably a 5-membered ring or a 6-membered ring. Further, the ring may contain a heteroatom (for example, an oxygen atom, a nitrogen atom, or a sulfur atom) in addition to the nitrogen atom to which R₂i to R₂₈ are bonded.

R₂₉ represents a substituent, where this substituent is the same as that described as the substituent described above in a case where R₂₁ to R₂₈ represent a substituent.

R₂₉ preferably represents an ionic hydrophilic group or a substituted or unsubstituted alkyl group. The alkyl group is preferably an alkyl group having 1 to 12 carbon atoms, more preferably an alkyl group having 1 to 8 carbon atoms, and most preferably an alkyl group having 1 to 6 carbon atoms.

In a case where a plurality of R₂₉'s, are present, the plurality of R₂₉'s may be the same or different from each other. In a case where a plurality of R₂₉'s are present, the plurality of R₂₉'s may be bonded to form a ring. The ring is not particularly limited; however, it may be an aromatic ring or a non-aromatic ring, and it is preferably a 5-membered ring or a 6-membered ring. Further, the ring may contain a heteroatom (for example, an oxygen atom, a nitrogen atom, or a sulfur atom).

At least one of R₂₁ to R₂₉ has a hydrophilic group. In a case of a compound having three or more aromatic rings in one molecule, it is particularly preferable that the compound has at least two hydrophilic groups bonded to the aromatic rings in the molecule.

The hydrophilic group can be easily selected as a substituent from the calculated value I/O of {O: organicity value} and {I: inorganicity value}, outlined in New Organic Conceptual Diagram: Basics and Applications (Sankyo Publishing Co., Ltd.), or from the logP value (generally, the molecular partition coefficient P in a 1-octanol/water system) widely used in the fields of chemistry and medicine as the value of hydrophobicity parameter of the compound or ClogP value, which is the calculated value of the logP value, and the acid dissociation constant (the pKa value) of the functional group. Further, the hydrophilic group is also preferably the “group having high hydrophilicity” and the “group not having high hydrophilicity” in the description of the “hydrophilic group” in Chemical Dictionary, the 4th edition (Kyoritsu Shuppan Co., Ltd.). Since the dye ink composition (the ink for ink jet) according to the embodiment of the present invention is preferably used in a basic manner, a hydrophilic group having a high acid dissociation constant (pKa value) and a group not having high hydrophilicity can also be applied. Specific examples thereof include -NH2, —OH, and —CO₂H (or an alkali metal salt of a carboxy group).

Further preferred hydrophilic groups include, in addition to the ionic hydrophilic group, a hydroxy group, an alkylcarbonylamino group, an arylcarbonylamino group, an alkylsulfonylamino group, an arylsulfonylamino group, and a quaternary ammonium group, which are not limited thereto. The hydrophilic group is preferably an ionic hydrophilic group, more preferably a sulfo group (—SO₃R) or a carboxy group (—CO₂R), and most preferably a sulfo group (—SO₃R). The ionic hydrophilic group is the same as the ionic hydrophilic group in General Formula (I) described above.

R represents a hydrogen atom or a counter cation. The above R is the same as R in —SO₃R or —CO₂R as the ionic hydrophilic group in General Formula (I) described above.

The compound represented by General Formula (P) preferably has 1 to 10 hydrophilic groups in one molecule, and more preferably 2 to 8 hydrophilic groups therein.

The compound represented by General Formula (P) preferably has 2 to 6 ionic hydrophilic groups in one molecule, and more preferably 2 to 4 ionic hydrophilic groups therein.

At least one of R₂₁ to R₂₉ in General Formula (P) preferably has an ionic hydrophilic group and more preferably has —SO₃R. It is still more preferable that 2 to 6 of R₂₁ to R₂9 have —SO₃R, and it is particularly preferable that 2 to 4 of R₂₁ to R₂9 have —SO₃R.

In General Formula (P), Arₗo represents a benzene ring or a naphthalene ring, and it preferably represents a benzene ring.

In a case where Ar20 represents a benzene ring, k represents an integer of 0 to 4. It is preferably an integer of 0 to 2, and more preferably 0 or 1.

In a case where Ar20 represents a naphthalene ring, k represents an integer of 0 to 6.

It is preferably an integer of 0 to 4, more preferably an integer of 0 to 2, and still more preferably 0 or 1.

Specific examples of the compound represented by General Formula (P) are shown below, which are not limited thereto. Further, although the counter cation (R) of the ionic hydrophilic group (for example, —SO₃R or —CO₂R) is described in the form of a salt, it is not limited to a single salt. It may be a partially free acid (for example, R=a Li ion and a hydrogen atom, or a Na ion and a hydrogen atom), and a mixed salt (for example, R is a salt of a Li ion and a Na ion, or a salt of a Na ion and an NHL ion).

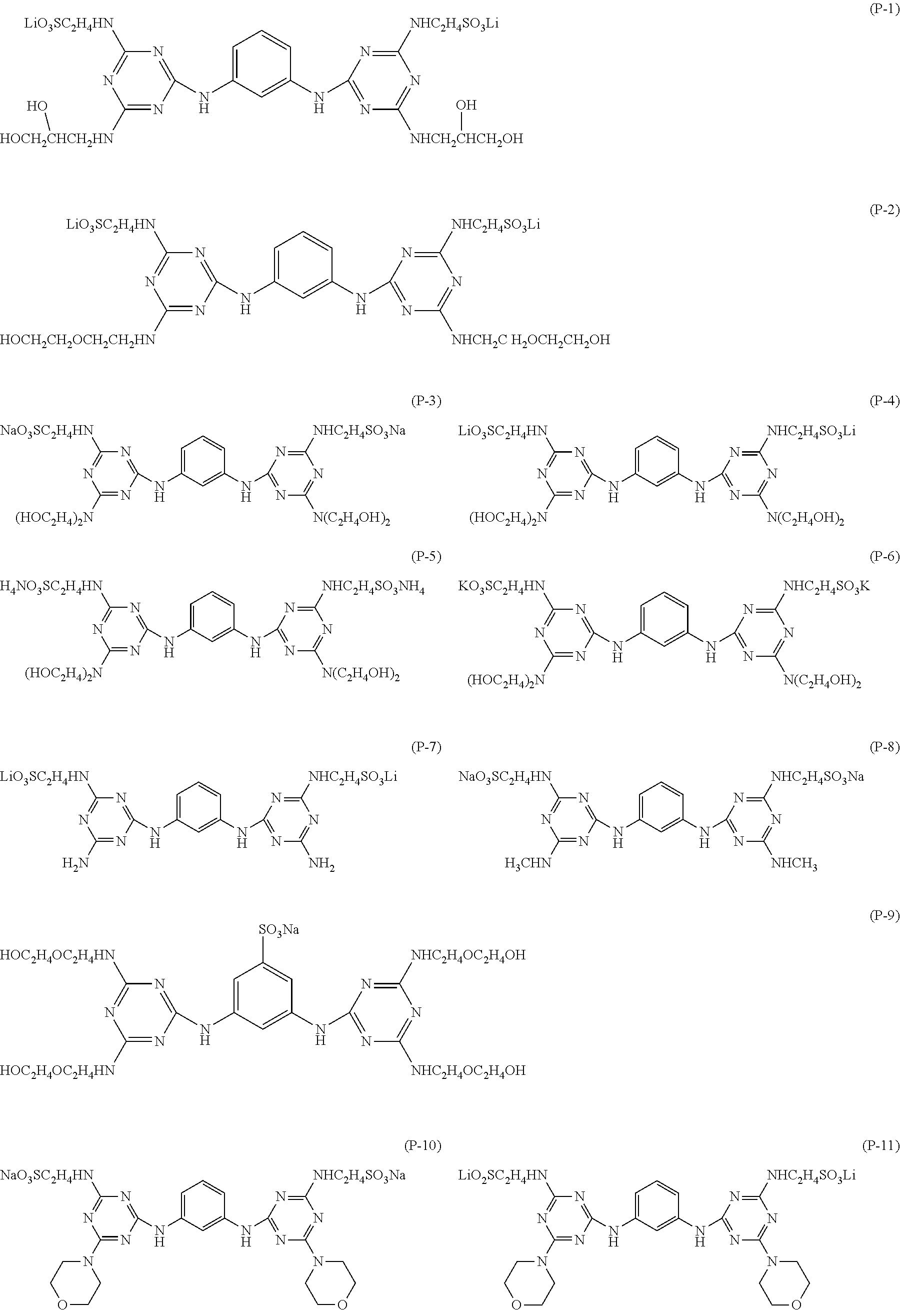

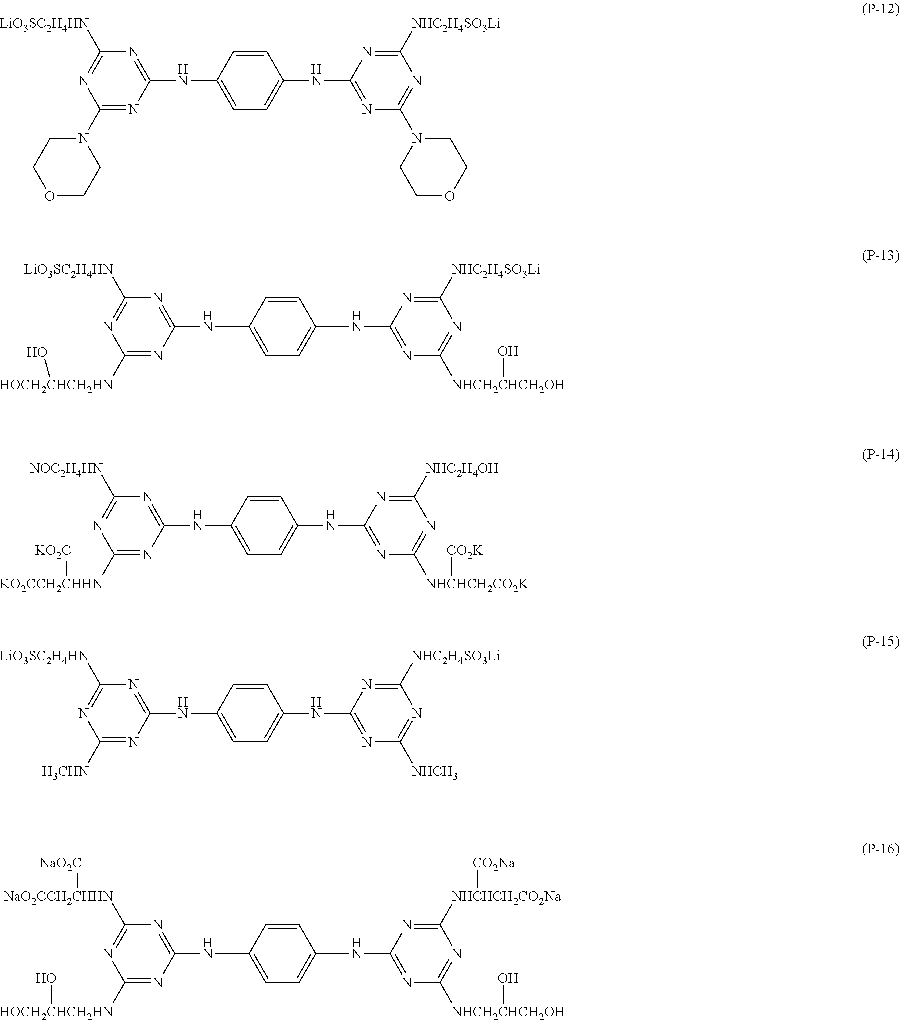

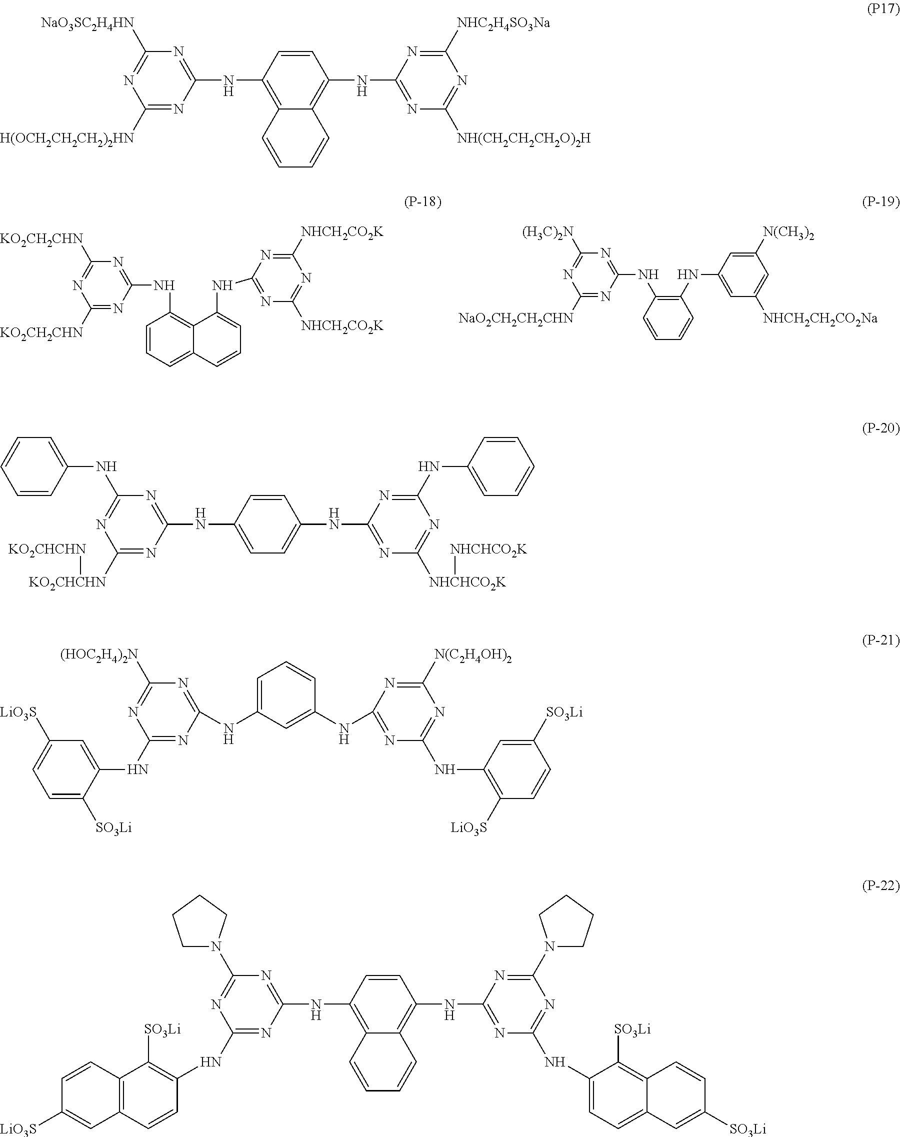

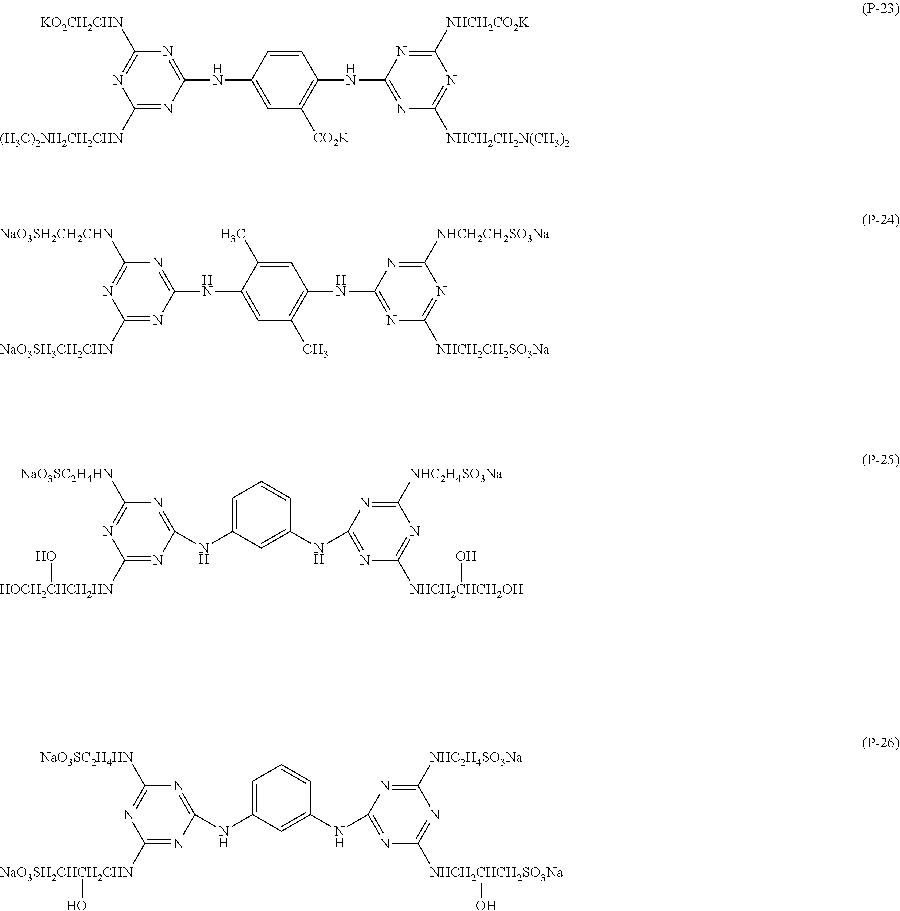

The content of the compound represented by the General Formula (P) in the dye ink composition according to the embodiment of the present invention is preferably 0.1% to 10.0°% o by mass, more preferably 0.3°% o to 5.0% by mass, still more preferably 0.5% to 4.0% by mass, particularly preferably 0.5% to 3.5% by mass, even still more preferably 0.5% to 3.0% by mass, and most preferably 0.5% to 2.5% by mass, based on the total mass of the dye ink composition.

The compound represented by General Formula (P) can be synthesized by a known method (for example, the method described in JP4686151B).

### Chelating Agent

The dye ink composition according to the embodiment of the present invention may contain a chelating agent.

The chelating agent (also referred to as a “chelating reagent”) is a compound that binds to an inorganic or metallic cation (particularly preferably a polyvalent cation) to generate a chelating compound.

In the present invention, the chelating agent has a function of preventing the formation and growth of insoluble precipitated foreign substances in a dye ink composition, derived from an inorganic or metal cation (particularly a polyvalent cation) (that is, functions as a solubilizing agent).

In a case of containing a chelating agent, the dye ink composition according to the embodiment of the present invention can suppress the generation of precipitated foreign substances even during long-term storage of the dye ink composition, and thus ink clogging at a nozzle or the like hardly occurs, and it is possible to obtain a high-quality printed material in a case of being used as the ink jet recording ink using the dye ink composition after long-term storage to carry out printing with an ink jet printer.

These days, the ink jet recording ink is in the process of changing from a cartridge ink to a large-capacity ink tank model, further improvement in storage stability (the printing density after long-term storage and the continuous ink jetting stability) during long-term storage is required, and the dye ink composition according to the embodiment of the present invention can further improve the storage stability during long-term storage in a case of containing a chelating agent.

As the chelating agent, various solubilizing agents can be used singly or in a combination of two or more as long as they have an effect of forming a complex with a cation present in the dye ink composition due to the chelating action thereof and suppressing the generation and growth of precipitated foreign substances in the dye ink composition; however, a water-soluble compound is preferable.

Examples of the chelating agent include ethylenediaminetetraacetic acid (EDTA) or a salt thereof (for example, tetrasodium EDTA (a tetrasodium salt) or tetralithium EDTA (a tetralithium salt)), picolinic acid or a salt thereof (for example, sodium picolinate), quinolinic acid or a salt thereof (for example, sodium quinolinate), 1,10-phenanthroline, 8-hydroxyquinoline, tetrasodium 3-hydroxy-2,2′-iminodisuccinate, methylglycine diacetic acid (MGDA) or a salt thereof, L-glutamic acid diacetic acid (GLDA) or a salt thereof, L-aspartic acid diacetic acid (ASDA) or a salt thereof, hydroxyethyliminodiacetic acid (HIDA) or a salt thereof, 3-hydroxy-2,2′-iminodisuccinic acid (HIDS) or a salt thereof, dicarboxymethylglutamic acid (CMGA) or a salt thereof, and (S,S)-ethylenediaminedisuccinic acid (EDDS) or a salt thereof. The salt among the above-described chelating agents is preferably a salt of ammonium, amine, or the like, in addition to a monovalent metal salt of sodium, potassium, lithium, and the like. Among the above-described chelating agents, these have further smaller attenuation of the chelating action with respect to the pH change of the dye ink composition. As a result, the chelating action is exhibited in a wider range of pH, and it is possible to improve, for example, the adaptability to the pH change of the dye ink composition, such as the temporal change.

The content of the chelating agent is preferably 0.001% by mass or more and 1.1% by mass or less, more preferably 0.001% by mass or more and 0.5% by mass, still more preferably 0.001% by mass or more and 0.3% by mass or less, and particularly preferably 0.001% by mass or more and 0.1% by mass or less, based on the total mass of the dye ink composition. In a case where it is 0.001% by mass or more, the chelating action can be effectively exhibited, and in a case where it is 1.1% by mass or less, it is possible to suppress the excessive increase in the viscosity or the excessive increase in the pH of the dye ink composition due to the addition of the chelating agent.

Further, the ratio of the chelating agent to the coloring agent (the content based on the mass of the chelating agent:the content based on the mass of the coloring agent) in the dye ink composition is preferably in a range of 0.0001:1 to 0.15:1 (“the chelating/coloring agent” is preferably 0.0001 to 0.15). It is more preferably in a range of 0.0001:1 to 0.01:1, and still more preferably in a range of 0.0002:1 to 0.005:1.

The metal that has a possibility to form a metal salt is a metal that has a possibility to be mixed during the dye production process or contained in the ink storage container of the dye ink composition and eluted into the dye ink composition. However, it is possible to effectively suppress the generation of foreign substances that causes clogging of the ink jet head at the above-described ratio, which is preferable. Further, it is preferable since the chelating action can be effectively exhibited, and it is possible to suppress the excessive increase in the viscosity or the excessive increase in the pH of the dye ink composition.

Examples of the use application of the dye ink composition according to the embodiment of the present invention include an image recording material for forming an image, and specifically, the use application is, including an ink jet method-type recording material described in detail below, a heat-sensitive recording material, a pressure-sensitive recording material, a recording materials using an electrophotographic method, a transfer-type silver halide photosensitive material, a printing ink, and a recording pen. It is preferably an ink jet method-type recording material, a heat-sensitive recording material, or a recording material using an electrophotographic method, and more preferably an ink jet method-type recording material.

Further, the dye ink composition according to the embodiment of the present invention can also be applied to a color filter for recording and reproducing a color image, which is used in a solid-state imaging element such as a charge-coupled device (CCD) or a display such as liquid crystal display (LCD) or a plasma display panel (PDP), as well as a staining solution for staining various fibers.

The dye ink composition according to the embodiment of the present invention can be used by adjusting, with a substituent, physical properties such as solubility, dispersibility, and heat transfer, suitable for the use application of the dye ink composition.

The dye ink composition according to the embodiment of the present invention is particularly suitable as an ink jet recording dye ink.

The ink jet recording dye ink according to the embodiment of the present invention has the above-described dye ink composition according to the embodiment of the present invention.

The dye ink composition according to the embodiment of the present invention preferably contains water.

The dye ink composition according to the embodiment of the present invention can be produced by using water as a medium, and as necessary, further using a lipophilic medium or aqueous medium to dissolve and/or dispersing a coloring agent, a toning agent, and an additive in the medium.

Examples of water include pure water such as deionized water, ion exchange water, ultrapure filtered water, reverse osmosis water, or distilled water, and ultrapure water.

The dye ink composition according to the embodiment of the present invention can contain the following organic solvents in addition to water. Examples of the organic solvent include alcohols (such as methanol, ethanol, propanol, isopropanol, butanol, isobutanol, sec-butanol, t-butanol, pentanol, hexanol, cyclohexanol, and benzyl alcohol), polyhydric alcohols (such as ethylene glycol, diethylene glycol, triethylene glycol, polyethylene glycol, propylene glycol, dipropylene glycol, polypropylene glycol, butylene glycol, hexanediol, pentanediol, glycerin, hexanetriol, and thiodiglycol), glycol derivatives (such as ethylene glycol monomethyl ether, ethylene glycol monoethyl ether, ethylene glycol monobutyl ether, diethylene glycol monomethyl ether, diethylene glycol monobutyl ether, propylene glycol monomethyl ether, propylene glycol monobutyl ether, dipropylene glycol monomethyl ether, triethylene glycol monomethyl ether, ethylene glycol diacetate, ethylene glycol monomethyl ether acetate, triethylene glycol monomethyl ether, triethylene glycol monoethyl ether, triethylene glycol monobutyl ether, and ethylene glycol monophenyl ether), amines (such as ethanolamine, di ethanol amine, triethanolamine, N-methyldiethanolamine, N-ethyldiethanolamine, morpholine, N-ethylmorpholine, ethylenediamine, diethylenetriamine, triethylenetetramine, polyethyleneimine, and tetramethylpropylenediamine), and other polar solvents (such as formamide, N,N-dimethylformamide, N,N-dimethylacetamide, dimethyl sulfoxide, sulfolane, 2-pyrrolidone, N-methyl-2-pyrrolidone, N-vinyl-2-pyrrolidone, 2-oxazolidone, ethylene urea, 1,3-dimethyl-2-imidazolidinone, acetonitrile, and acetone). Two or more kinds of organic solvents may be used in combination.

In a case where the dye ink composition according to the embodiment of the present invention contains an organic solvent, the content of the organic solvent is preferably 10°% o to 55% by mass, preferably 20% to 50% by mass, and still more preferably 30% to 45% by mass, based on the total mass of the dye ink composition.

In the dye ink composition according to the embodiment of the present invention, the water content is preferably 40% to 80% by mass, more preferably 45% to 70% by mass, and still more preferably 50% to 60% by mass, based on the total mass of the dye ink composition from the viewpoints of continuous jetting stability and storage stability of the ink.

The dye ink composition according to the embodiment of the present invention may contain other additives, as necessary, within a range that does not impair the effect of the present invention.

Examples of other additives include known additives (those described in JP2003-306623) such as an anti-drying agent (wetting agent), an antifading agent, an emulsion stabilizer, a penetration enhancer, an ultraviolet absorbing agent, a preservative, an antifungal agent, a pH adjusting agent, a surface tension adjuster, an antifoaming agent, a viscosity adjuster, a dispersing agent, a dispersion stabilizer, a rust inhibitor, and a betaine. These various additives can be added directly to the dye ink composition. As the preservative, the same preservative contained in the aqueous dye solution according to the embodiment of the present invention, which will be described later, can be used.

Examples of the surface tension adjuster include a nonionic, cationic, or anionic surfactant. Examples of the surfactant are preferably the following surfactants; anionic surfactants such as a fatty acid salt, an alkyl sulfate ester salt, an alkyl benzene sulfonate, an alkyl naphthalene sulfonate, a dialkyl sulfosuccinate, an alkyl phosphate ester salt, a naphthalene sulfonate formalin condensate, and a polyoxyethylene alkyl sulfuric acid ester salt; and nonionic surfactants such as a polyoxyethylene alkyl ether, a polyoxyethylene alkyl allyl ether, a polyoxyethylene fatty acid ester, a sorbitan fatty acid ester, a polyoxyethylene sorbitan fatty acid ester, a polyoxyethylene alkylamine, a glycerin fatty acid ester, and an oxyethyleneoxypropylene block copolymer. Further, SURFYNOL (registered trade name) series manufactured by Nissin Chemical Co., Ltd., which is an acetylene-based polyoxyethylene oxide surfactant, is also preferably used. Furthermore, an amine oxide-type amphoteric surfactant or the like such as N,N-dimethyl-N-alkylamine oxide is also preferable. Furthermore, it is possible to use those exemplified as surfactants on pages 37 and 38 of JP1984-157636A (JP-H59-157636A), and Research Disclosure No. 308119 (1989).

### Physical Properties of Dye Ink Composition

The surface tension of the dye ink composition according to the embodiment of the present invention at 25° C. is preferably 10 mN/m or more and 60 mN/m or less, more preferably 20 mN/m or more and 60 mN/m or less, and still more preferably 30 mN/m or more and 40 mN/m. In a case where the surface tension of the dye ink composition according to the embodiment of the present invention is set within the above range, it is possible to effectively suppress the occurrence of jetting shift (the deviation of the ink landing point) due to wetting in the vicinity of the outlet in a case of being applied to the ink jet method. The surface tension of the ink can be adjusted by appropriately determining the content of the surfactant or the like in the dye ink composition. Further, the dye ink composition according to the embodiment of the present invention is preferably adjusted to a desired pH so that good jetting characteristics can be obtained in a case of being applied to an ink jet recording device. The viscosity of the dye ink composition according to the embodiment of the present invention at 25° C. is preferably 1.0 mPa·s or more and 5.0 mPa·s or less.

### Ink Jet Recording Method

The ink jet recording method according to the embodiment of the present invention includes a step of jetting the ink jet recording dye ink according to the embodiment of the present invention by using an ink jet method-type recording head. More specifically, the ink jet recording method according to the embodiment of the present invention is a recording method of forming an image, by providing energy to the ink jet recording dye ink according to the embodiment of the present invention, on a known image receiving materials, that is, plain paper or resin coated paper, for example, paper exclusive for ink jet described in JP1996-169172A (JP-H8-169172A), JP1996-27693A (JP-H8-27693A), JP1990-276670A (JP-H2-276670A), JP1995-276789A (JP-H7-276789A), JP1997-323475A (JP-H9-323475A), JP1987-238783A (JP-S62-238783A), JP1998-153989A (JP-H10-153989A), JP1998-217473A (JP-H10-217473A), JP1998-235995A (JP-H10-235995A), JP1998-337947A (JP-H10-337947A), JP1998-217597A (JP-H10-217597), JP1998-337947A (JP-H10-337947), or the like, a film, common electrophotographic paper, cloth, glass, metal, or ceramics.

In a case of forming an image, a polymer latex compound may be added for the intended purpose of imparting glossiness and water resistance or improving weather fastness.

The recording system of the ink jet recording method according to the embodiment of the present invention is not limited, and it is used in a known method, for example, an electric charge control method of jetting ink by using an electrostatic attraction force; a drop-on-demand method (pressure pulse method) of using a vibration pressure of a piezo element; an acoustic ink jet method of converting an electric signal into an acoustic beam, irradiating ink, and jetting the ink using a radiation pressure; and a thermal ink jet method of heating ink to form air bubbles and utilizing the generated pressure. Examples of the ink jet recording system include a method of jetting a small volume of an ink having a low density, which is referred to as a photo ink, multiple times, a method of improving the image quality using a plurality of inks having substantially the same color tone and different densities, and a method of using a colorless transparent ink.

In the ink jet recording method according to the embodiment of the present invention, an ink jet recording dye ink containing the dye ink composition according to the embodiment of the present invention described above can be jetted by an ink jet method-type recording head to record an image on a recording medium. Further, in addition to the dye ink composition according to the embodiment of the present invention described above (preferably a cyan dye ink composition), a magenta ink composition, a yellow ink composition, and a black ink composition can be used as an ink set.

Since the dye ink composition and the ink jet recording dye ink according to the embodiment of the present invention are excellent in storage stability, they can be applied to an ink jet printer equipped with a large-capacity ink tank.

### Aqueous Dye Solution

The aqueous dye solution according to the embodiment of the present invention is an aqueous dye solution containing;
a preservative,
in which the aqueous dye solution contains a phthalocyanine compound represented by the following General Formula (I), and a content of the phthalocyanine compound represented by the General Formula (I) is 8% to 15% by mass with respect to a total mass of the aqueous dye solution.

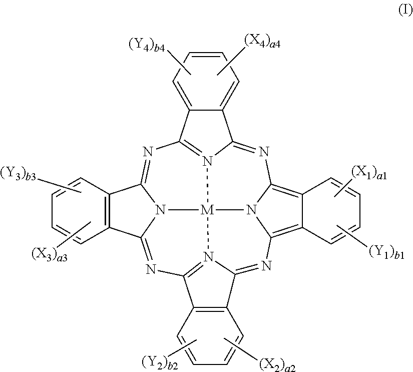

in the General Formula (I),
X₁, X₂, X₃, and X₄ each independently represent —N═N—Cp,
Cp represents an organic group,
Y₁, Y₂, Y₃, and Y₄ each independently represent a substituent,
at least one of X₁, X₂, X₃, X₄, Y₁, Y₂, Y₃, or Y₄ contains an ionic hydrophilic group,
a1, a2, a3, a4, b1, b2, b3, and b4 each independently represent an integer of 0 to 4,
a1 +b 1, a2+b2, a3+b3, and a₄+b4 are each independently an integer of 0 to 4,
all of a1, a2, a3, and a4 are not 0 at the same time, and
M represents a hydrogen atom, a metal element, a metal oxide, a metal hydroxide, or a metal halide.

The phthalocyanine compound represented by General Formula (I) in the aqueous dye solution according to the embodiment of the present invention is the same as that in the dye ink composition according to the embodiment of the present invention described above.

The content of the phthalocyanine compound represented by General Formula (I) in the aqueous dye solution according to the embodiment of the present invention is 8% to 15% by mass, preferably 10% to 15% by mass, and more preferably 10% to 12% by mass, with respect to the total mass of the aqueous dye solution. Since the aqueous dye solution according to the embodiment of the present invention contains a phthalocyanine compound represented by General Formula (I) at a high concentration, the dye ink composition according to the embodiment of the present invention can be easily prepared by diluting the aqueous dye solution according to the embodiment of the present invention with a solvent (at least one solvent of water or the above organic solvent).

### Preservative

Next, a preservative will be described.

In the present invention, the preservative refers to an agent having a function of preventing the outbreak and growth of microorganisms, particularly bacteria and fungi (molds).

In a case of using a preservative for the aqueous dye solution according to the embodiment of the present invention, it is possible to suppress the outbreak of molds even during the long-term storage of the aqueous dye solution, and thus ink clogging at a nozzle or the like hardly occurs, and it is possible to obtain a high-quality printed material in a case of carrying out printing with an ink jet printer by using an ink jet recording ink using the aqueous dye solution after long-term storage.

As the preservative that can be used in the present invention, various preservatives can be used.

Examples of the preservative include an inorganic preservative containing a heavy metal ion and an organic preservative. As the organic preservatives, it is possible to use various preservatives such as a quaternary ammonium salt (tetrabutylammonium chloride, cetylpyridinium chloride, benzyltrimethylammonium chloride, or the like), phenols (phenol, cresol, butylphenol, xylenol, bisphenol, and the like), a phenoxyether derivative (phenoxyethanol or the like),a heterocyclic compound (benzotriazole, 1,2-benzisothiazolin-3-one, sodium dehydroacetate, PROXEL (registered trade name) series manufactured by Lonza Group AG, or the like), alkanediols (pentylene glycol (1,2-pentanediol), i sopentyl di ol (3-methyl-1,3-butanediol), hexanediol (1,2-hexanediol), or the like), caprylyl glycol (1,2-octanediol) or the like), acid amides, carbamic acid, carbamates, amidines/guanidines, pyridines (sodium pyridinethion-1-oxide or the like), diazines, triazines, pyrroles/imidazoles, oxazoles/oxazines, thiazoles/thiadiazines, thioureas, thiosemicarbazides, dithiocarbamates, sul tides, sul foxi des, sulfones, sulfami des, antibiotics (penicillin, tetracycline, and the like), aromatic carboxylic acids and salts thereof (sodium benzoate and the like), and aromatic carboxylic acid esters and salts thereof (p-hydroxybenzoic acid ethyl ester and the like).

The preservative is preferably at least one selected from the group consisting of a heterocyclic compound, phenols, a phenoxyether derivative, and alkanediols, and more preferably a heterocyclic compound.

Further, as the preservative, those described in Antibacterial and Antifungal Handbook (GIHODO SHUPPAN Co., Ltd.: 1986), Antibacterial and Antifungal Agent Dictionary (edited by Dictionary Editorial Committee, the Society for Antibacterial and Antifungal Agents, Japan), and the like can also be used.

Various compounds such as those having an oil-soluble structure and those having a water-soluble structure can be used as these compounds; however, a water-soluble compound is preferable.

The aqueous dye solution according to the embodiment of the present invention may contain two or more kinds of preservatives.

The heterocyclic compound is preferably a thiazole-based compound or a benzotriazole-based compound.

Particularly, the thiazole-based compound among the preservatives functions as an antifungal agent. Examples of the thiazole compound include benzisothiazoline, isothiazoli ne, 1,2-benzisothiazolin-3-one, 5-chloro-2-methyl -4-i sothiazolin-3-one, 2-(thiocyanomethylthio)benzothiazole, 2-mercaptobenzothiazole, and 3-allyloxy-1,2-benzisothiazol-1,1-oxide. Further, as a thiazole-based antifungal agent, a PROXEL (registered trade name) series (BDN, BD20, GXL, LV, XL2, XL2(s), Ultra10, etc.) manufactured by Lonza Group AG can also be used.

Among the preservatives, the benzotriazole-based compound functions particularly as a rust inhibitor, and it is possible to prevent, for example, the occurrence of rust caused by the contact between an ink and a metal material that constitutes an ink jet head (particularly, 42 alloy (a nickel-iron alloy containing 42% of nickel)). Examples of the benzotriazole-based compound include 1H-benzotriazole, 4-methyl-1H-benzotriazole, 5-methyl-1H-benzotriazole, and sodium salts or potassium salts thereof.

The content of the preservative in the aqueous dye solution according to the embodiment of the present invention can be used in a wide range. However, it is preferably 0.001% to 10% by mass, more preferably 0.005% to 2.0% by mass, still more preferably 0.01% to 0.5% by mass, and particularly preferably 0.01% to 0.1% by mass, with respect to the total amount of the aqueous dye solution. In a case where it is set to 0.001% to l0% by mass, the effect of the preservative can be obtained more efficiently, and the generation of precipitates can be suppressed.

The aqueous dye solution according to the embodiment of the present invention may further contain a chelating agent. As the chelating agent, the same chelating agent as the chelating agent described above as the chelating agent that may be contained in the dye ink composition according to the embodiment of the present invention can be used.

In a case where the aqueous dye solution according to the embodiment of the present invention contains a chelating agent, the preferred range of the content of the chelating agent is the same as the preferred range of the content of the chelating agent in a case where the above-described dye ink composition according to the embodiment of the present invention contains the chelating agent.

The aqueous dye solution according to the embodiment of the present invention may further contain a toning dye. The toning dye is the same as that in the dye ink composition according to the embodiment of the present invention described above.

In a case where the aqueous dye solution according to the embodiment of the present invention contains a toning dye, the preferred range of the content of the toning dye is the same as the preferred range of the content of the toning dye in a case where the above-described dye ink composition according to the embodiment of the present invention contains the toning dye.

The aqueous dye solution according to the embodiment of the present invention preferably further contains the compound represented by General Formula (P) described above. The compound represented by General Formula (P) is the same as that in the dye ink composition according to the embodiment of the present invention described above.

In a case where the aqueous dye solution according to the embodiment of the present invention contains a compound represented by General Formula (P), the preferred range of the content of the compound represented by General Formula (P) is the same as the preferred range of the content of the compound represented by General Formula (P) in a case where the above-described dye ink composition according to the embodiment of the present invention contains the compound represented by General Formula (P).

### EXAMPLES

Hereinafter, the present invention will be described in detail with reference to Examples; however, the present invention is not limited to these Examples.

### Example 1

Deionized water was added to a mixture containing the following components in the following adding amounts, respectively, to make the mixture 100 g, and the mixture was stirred for 1 hour while heating at 30° C. to 40° C. Then, the pH was adjusted to 9.0 with a 10 mol/L sodium hydroxide aqueous solution, and filtration was carried out under reduced pressure with a microfilter having an average pore diameter of 0.25 gm to prepare a black dye ink (BK-1).

| | |
|---|---|
| Coloring agent (BLACK dye 1) | 5.00 g |
| Glycerin | 10.00 g |
| Triethylene glycol | 2.00 g |
| Triethylene glycol monobutyl ether | 10.00 g |
| 2-pyrrolidone | 2.00 g |
| Surfactant | 1.00 g |
| Preservative | 0.11 g |

The above surfactant is SURFYNOL (registered trade name) 465 manufactured by Nisshin Chemical Industry Co., Ltd. The same applies to the surfactants in the following Examples and Comparative Examples.

The above preservative is PROXEL XL2(s) (registered trade name) manufactured by Lonza Group AG. The same applies to the preservatives in the following Examples and Comparative Examples.

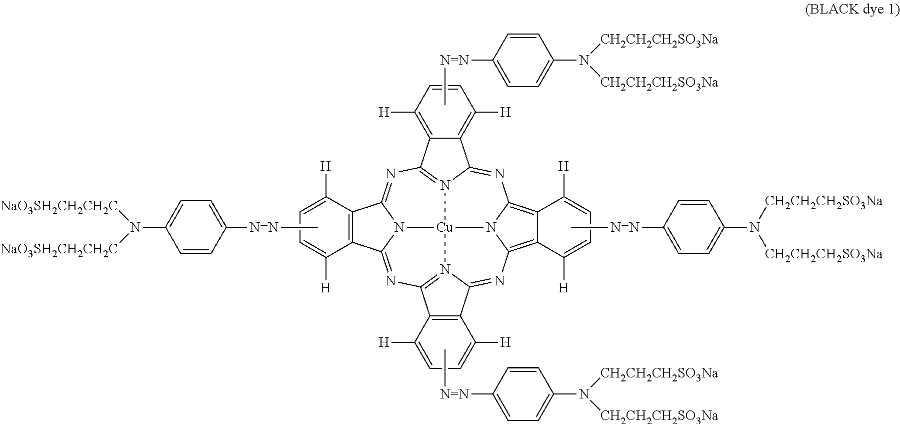

### Example 2

Deionized water was added to a mixture containing the following components in the following adding amounts, respectively, to make the mixture 100 g, and the mixture was stirred for 1 hour while heating at 30° C. to 40° C. Then, the pH was adjusted to 9.0 with a 10 mol/L sodium hydroxide aqueous solution, and filtration was carried out under reduced pressure with a microfilter having an average pore diameter of 0.25 gm to prepare a black dye ink (BK-2).

| | |
|---|---|
| Coloring agent (BLACK dye 1) | 4.00 g |
| Toning agent (toning dye 1) | 1.00 g |
| Glycerin | 10.00 g |
| Tri ethylene glycol | 2.00 g |
| Triethylene glycol monobutyl ether | 10.00 g |
| 2-pyrrolidone | 2.00 g |
| Surfactant | 1.00 g |
| Preservative | 0.11 g |

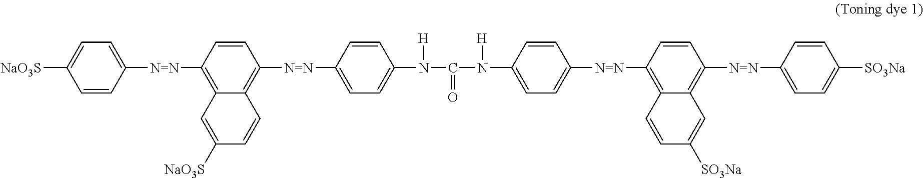

### Example 3

Deionized water was added to a mixture containing the following components in the following adding amounts, respectively, to make the mixture 100 g, and the mixture was stirred for 1 hour while heating at 30° C. to 40° C. Then, the pH was adjusted to 9.0 with a 10 mol/L sodium hydroxide aqueous solution, and filtration was carried out under reduced pressure with a microfilter having an average pore diameter of 0.25 pm to prepare a black dye ink (BK-3).

| | |
|---|---|
| Coloring agent (BLACK dye 1) | 3.50 g |
| Toning agent (toning dye 1) | 1.00 g |
| Toning agent (toning dye 2) | 0.50 g |
| Glycerin | 10.00 g |
| Triethylene glycol | 2.00 g |
| Triethylene glycol monobutyl ether | 10.00 g |
| 2-pyrrolidone | 2.00 g |
| Surfactant | 1.00 g |
| Preservative | 0.11 g |

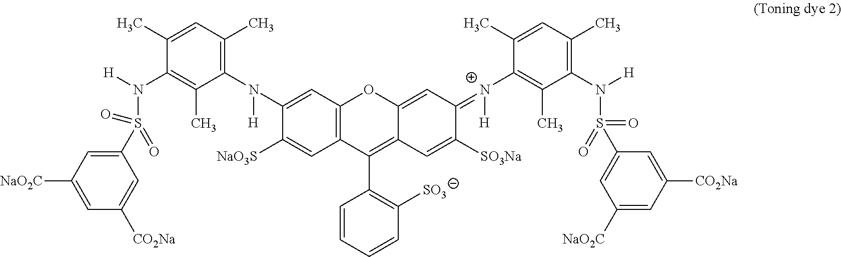

### Example 4

Deionized water was added to a mixture containing the following components in the following adding amounts, respectively, to make the mixture 100 g, and the mixture was stirred for 1 hour while heating at 30° C. to 40° C. Then, the pH was adjusted to 9.0 with a 10 mol/L sodium hydroxide aqueous solution, and filtration was carried out under reduced pressure with a microfilter having an average pore diameter of 0.25 pm to prepare a black dye ink (BK-4).

| | |
|---|---|
| Coloring agent (BLACK dye 1) | 3.50 g |
| Toning agent (toning dye 1) | 1.00 g |
| Toning agent (toning dye 2) | 0.50 g |
| Additive (P-3) | 0.35 g |
| Glycerin | 10.00 g |
| Triethylene glycol | 2.00 g |
| Triethylene glycol monobutyl ether | 10.00 g |
| 2-pyrrolidone | 2.00 g |
| Surfactant | 1.00 g |
| Preservative | 0.11 g |

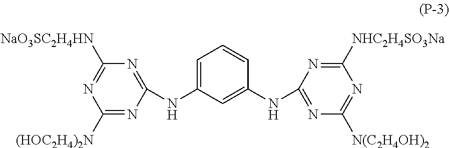

### Example 5

Deionized water was added to a mixture containing the following components in the following adding amounts, respectively, to make the mixture 100 g, and the mixture was stirred for 1 hour while heating at 30° C. to 40° C. Then, the pH was adjusted to 9.0 with a 10 mol/L lithium hydroxide aqueous solution, and filtration was carried out under reduced pressure with a microfilter having an average pore diameter of 0.25 gm to prepare a black dye ink (BK-5).

| | |
|---|---|
| Coloring agent (BLACK dye 2) | 3.50 g |
| Toning agent (toning dye 3) | 1.00 g |
| Toning agent (toning dye 2) | 0.50 g |
| Additive (P-4) | 0.35 g |
| Glycerin | 10.00 g |
| Triethylene glycol | 2.00 g |
| Triethylene glycol monobutyl ether | 10.00 g |
| 2-pyrrolidone | 2.00 g |
| Surfactant | 1.00 g |
| Preservative | 0.11 g |

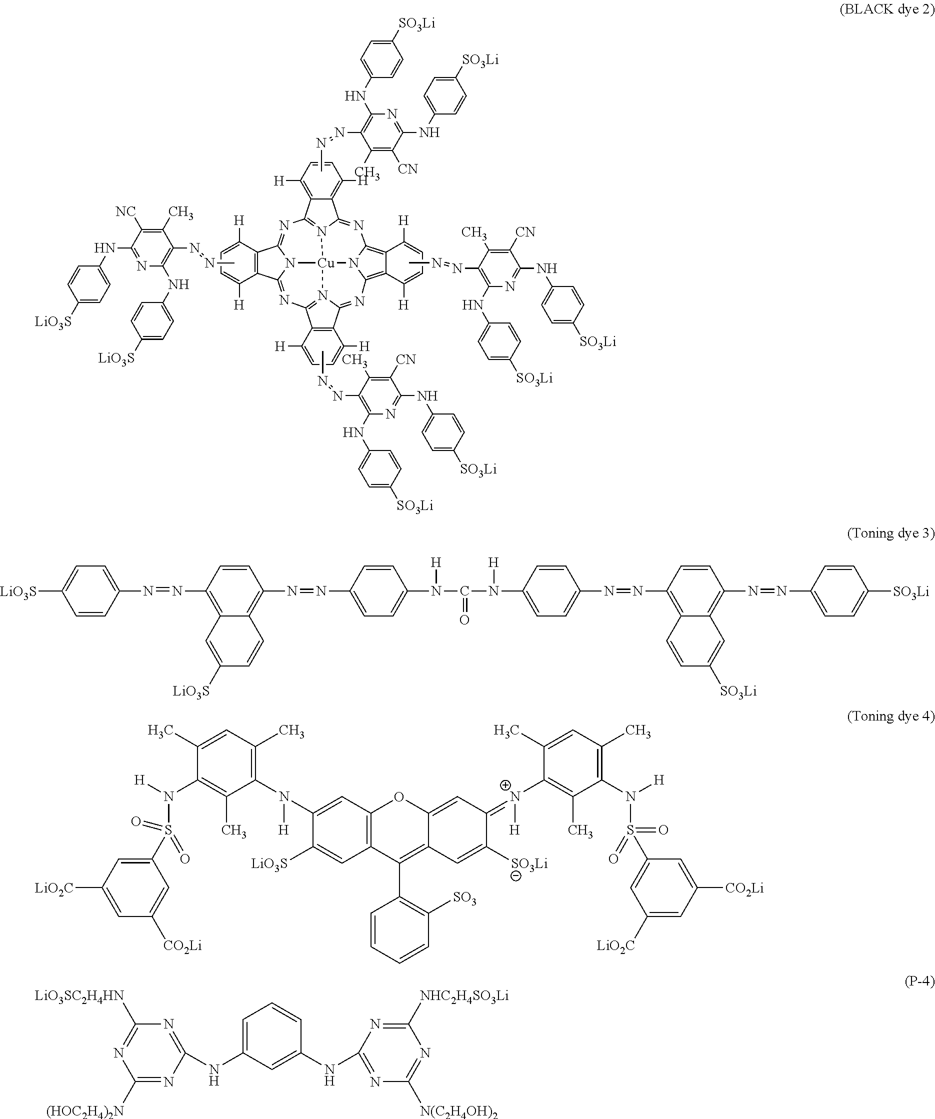

### Example 6

Deionized water was added to a mixture containing the following components in the following adding amounts, respectively, to make the mixture 100 g, and the mixture was stirred for 1 hour while heating at 30° C. to 40° C. Then, the pH was adjusted to 9.0 with a 10 mol/L lithium hydroxide aqueous solution, and filtration was carried out under reduced pressure with a microfilter having an average pore diameter of 0.25 μm to prepare a black dye ink (BK-6).

| | |
|---|---|
| Coloring agent (BLACK dye 3) | 3.50 g |
| Toning agent (toning dye 3) | 1.00 g |
| Toning agent (toning dye 4) | 0.50 g |
| Additive (P-4) | 0.35 g |
| Glycerin | 10.00 g |
| Triethylene glycol | 2.00 g |
| Triethylene glycol monobutyl ether | 10.00 g |
| 2-pyrrolidone | 2.00 g |
| Surfactant | 1.00 g |
| Preservative | 0.11 g |

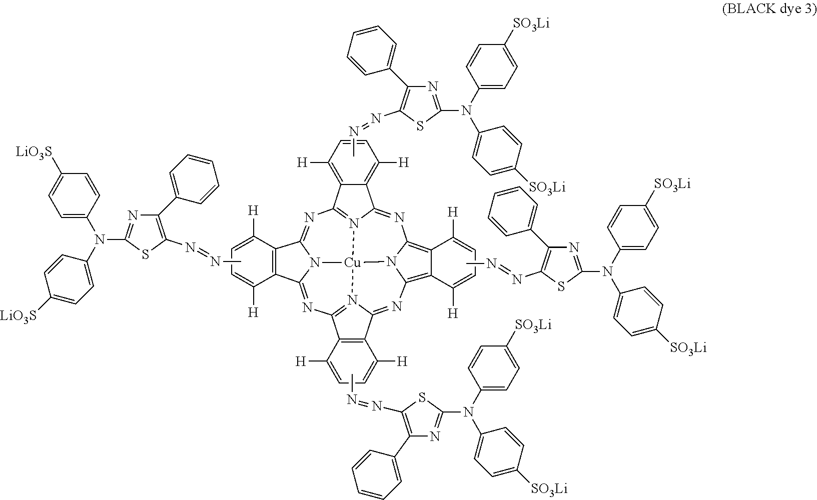

### Example 7

Deionized water was added to a mixture containing the following components in the following adding amounts, respectively, to make the mixture 100 g, and the mixture was stirred for 1 hour while heating at 30° C. to 40° C. Then, the pH was adjusted to 9.0 with a 10 mol/L sodium hydroxide aqueous solution, and filtration was carried out under reduced pressure with a microfilter having an average pore diameter of 0.25 pm to prepare a black dye ink (BK-7).

| | |
|---|---|
| Coloring agent (BLACK dye 4) | 3.50 g |
| Toning agent (toning dye 1) | 1.00 g |
| Toning agent (toning dye 2) | 0.50 g |
| Additive (P-3) | 0.35 g |
| Glycerin | 10.00 g |
| Triethylene glycol | 2.00 g |
| Triethylene glycol monobutyl ether | 10.00 g |
| 2-pyrrolidone | 2.00 g |
| Surfactant | 1.00 g |
| Preservative | 0.11 g |

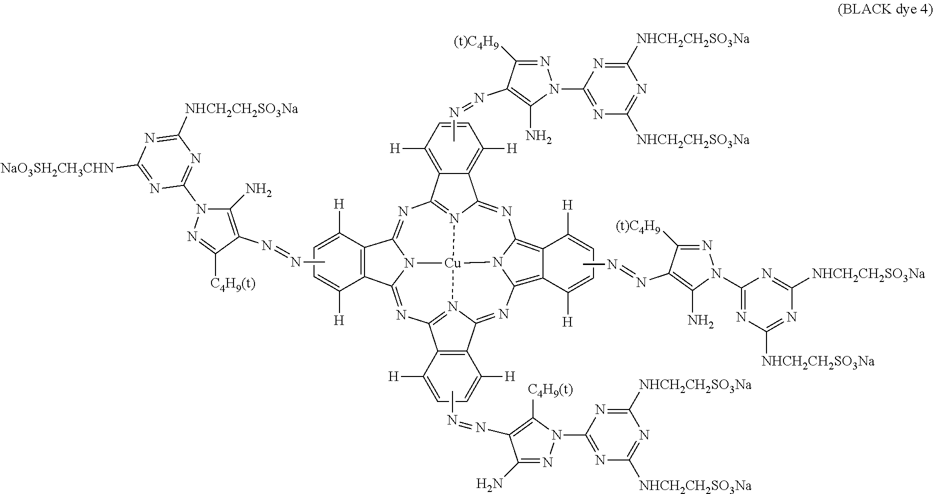

### Example 8

Deionized water was added to a mixture containing the following components in the following adding amounts, respectively, to make the mixture 100 g, and the mixture was stirred for 1 hour while heating at 30° C. to 40° C. Then, the pH was adjusted to 9.0 with a 10 mol/L lithium hydroxide aqueous solution, and filtration was carried out under reduced pressure with a microfilter having an average pore diameter of 0.25 gm to prepare a black dye ink (BK-8).

| | |
|---|---|
| Coloring agent (BLACK dye 2) | 3.00 g |
| Coloring agent (BLACK dye 5) | 1.00 g |
| Toning agent (toning dye 3) | 0.50 g |
| Toning agent (toning dye 4) | 0.50 g |
| Additive (P-4) | 0.30 g |
| Glycerin | 10.00 g |
| Triethylene glycol | 2.00 g |
| Triethylene glycol monobutyl ether | 10.00 g |
| 2-pyrrolidone | 2.00 g |
| Surfactant | 1.00 g |
| Preservative | 0.11 g |

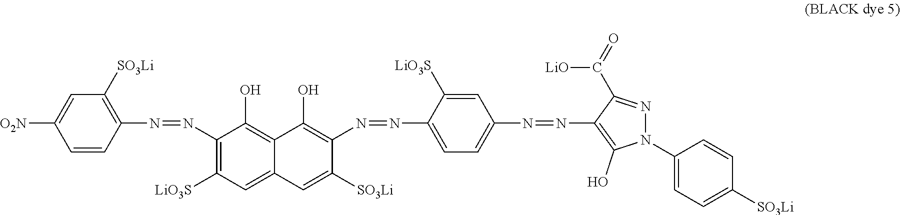

### Example 9

Deionized water was added to a mixture containing the following components in the following adding amounts, respectively, to make the mixture 100 g, and the mixture was stirred for 1 hour while heating at 30° C. to 40° C. Then, the pH was adjusted to 9.0 with a 10 mol/L lithium hydroxide aqueous solution, and filtration was carried out under reduced pressure with a microfilter having an average pore diameter of 0.25 pm to prepare a black dye ink (BK-9).

| | |
|---|---|
| Coloring agent (BLACK dye 2) | 3.00 g |
| Coloring agent (BLACK dye 6) | 1.00 g |
| Toning agent (toning dye 3) | 0.50 g |
| Toning agent (toning dye 4) | 0.50 g |
| Additive (P-4) | 0.30 g |
| Glycerin | 10.00 g |
| Triethylene glycol | 2.00 g |
| Triethylene glycol monobutyl ether | 10.00 g |
| 2-pyrrolidone | 2.00 g |
| Surfactant | 1.00 g |
| Preservative | 0.11 g |

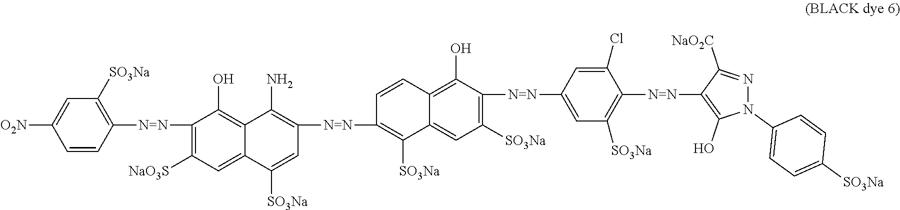

### Example 10

Deionized water was added to a mixture containing the following components in the following adding amounts, respectively, to make the mixture 100 g, and the mixture was stirred for 1 hour while heating at 30° C. to 40° C. Then, the pH was adjusted to 9.0 with a 10 mol/L lithium hydroxide aqueous solution, and filtration was carried out under reduced pressure with a microfilter having an average pore diameter of 0.25 gm to prepare a black dye ink (BK-10).

| | |
|---|---|
| Coloring agent (BLACK dye 2) | 3.00 g |
| Coloring agent (BLACK dye 7) | 1.00 g |
| Toning agent (toning dye 3) | 0.50 g |
| Toning agent (toning dye 2) | 0.50 g |
| Additive (P-4) | 0.30 g |
| Glycerin | 10.00 g |
| Triethylene glycol | 2.00 g |
| Triethylene glycol monobutyl ether | 10.00 g |
| 2-pyrrolidone | 2.00 g |
| Surfactant | 1.00 g |
| Preservative | 0.11 g |

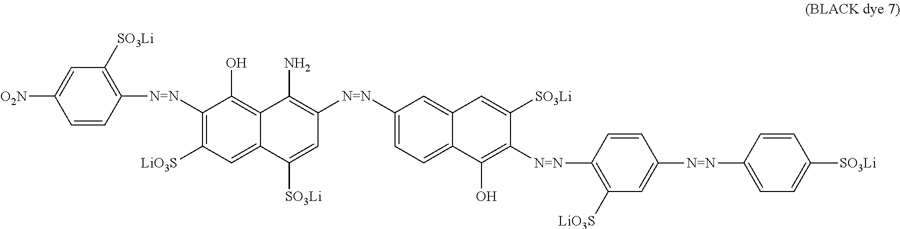

### Comparative Example 1

Deionized water was added to a mixture containing the following components in the following adding amounts, respectively, to make the mixture 100 g, and the mixture was stirred for 1 hour while heating at 30° C. to 40° C. Then, the pH was adjusted to 9.0 with a 10 mol/L lithium hydroxide aqueous solution, and filtration was carried out under reduced pressure with a microfilter having an average pore diameter of 0.25 μm to prepare a black dye ink (BK-1X).

| | |
|---|---|
| Coloring agent (BLACK dye 5) | 5.00 g |
| Glycerin | 10.00 g |
| Triethylene glycol | 2.00 g |
| Triethylene glycol monobutyl ether | 10.00 g |
| 2-pyrrolidone | 2.00 g |
| Surfactant | 1.00 g |
| Preservative | 0.11 g |

### Comparative Example 2

Deionized water was added to a mixture containing the following components in the following adding amounts, respectively, to make the mixture 100 g, and the mixture was stirred for 1 hour while heating at 30° C. to 40° C. Then, the pH was adjusted to 9.0 with a 10 mol/L lithium hydroxide aqueous solution, and filtration was carried out under reduced pressure with a microfilter having an average pore diameter of 0.25 pm to prepare a black dye ink (BK-2X).

| | |
|---|---|
| Coloring agent (BLACK dye 6) | 5.00 g |
| Glycerin | 10.00 g |
| Triethylene glycol | 2.00 g |
| Triethylene glycol monobutyl ether | 10.00 g |
| 2-pyrrolidone | 2.00 g |
| Surfactant | 1.00 g |
| Preservative | 0.11 g |

### Comparative Example 3

Deionized water was added to a mixture containing the following components in the following adding amounts, respectively, to make the mixture 100 g, and the mixture was stirred for 1 hour while heating at 30° C. to 40° C. Then, the pH was adjusted to 9.0 with a 10 mol/L lithium hydroxide aqueous solution, and filtration was carried out under reduced pressure with a microfilter having an average pore diameter of 0.25 pm to prepare a black dye ink (BK-3X).

| | |
|---|---|
| Coloring agent (BLACK dye 7) | 5.00 g |
| Glycerin | 10.00 g |
| Tri ethylene glycol | 2.00 g |
| Triethylene glycol monobutyl ether | 10.00 g |
| 2-pyrrolidone | 2.00 g |
| Surfactant | 1.00 g |
| Preservative | 0.11 g |

### Image Recording and Evaluation

Using the black dye inks (BK-1 to BK-10, and BK-1X to BK-3X) prepared as above, image recording was carried out with the combination of the ink jet printer and the recording paper shown below and evaluated.

In each of Examples and Comparative Examples, each black dye ink was loaded into an ink cartridge, an image was recorded on photo paper with an ink jet printer (manufactured by Seiko Epson Corporation; PX-045A) (photo paper <Glossy type>, manufactured by Seiko Epson Co., Ltd., and the image was used for evaluation.

### Color Tone

The printed sample was visually observed and evaluated in terms of the following four stages.

A: A range from neutral gray to black

B: A range from gray slightly close to reddish yellow to black, a range from gray slightly close to red to black, or a range from gray slightly close to blue to black

C: A range from gray close to reddish yellow to black, a range from gray close to red to black, or a range from gray close to blue to black (a level in which fine toning is required in terms of the color space when the density is measured)

D: A color that clearly deviates from gray (a level in which further toning is required)

From the point of view of the color tone of black, A to C are excellent, A or B is more excellent, and A is most excellent.

### Printing Density (Adhesion)

A solidly painted black image (a printed image at an applied voltage of 100%) was recorded using each black dye ink with the combination of the above-described ink jet printer and recording paper.

The printing density of the obtained solidly painted black image was measured using a reflection densitometer (product name: i 1 PUBLISH PRO 2, manufactured by X-Rite, Inc.), and it was evaluated in terms of the four stages by denoting a case where the printing density (Optical Density, OD) in a case of using a visual filter is 2.4 as A, a case where it is 2.2 or more and less than 2.4 as B, a case where it is 2.0 or more and less than 2.2 as C, and a case where it is less than 2.0 as D.

### Light Resistance

Using each black dye ink, a stair patch image of toning gray was printed, and at the time when 24 hours had passed at 25° C., (L3, a₃, b3) of the reflection density (OD=1.0) was measured from each density range of the stair patch portion by using an it PUBLISH PRO 2 densitometer originally equipped with a status A filter (Visual). Using a low-temperature cycle xenon weather meter (XL75 manufactured by Suga Test Instruments Co., Ltd.), this image sample was irradiated with xenon light (100,000 lux) for 14 days, (IA, a₄, b4) of the position having the initial reflection density (OD≈1.0) was measured, and the color difference (ΔE) was calculated as an indicator of discoloration/fading after the exposure test.

ΔE between (L3, a₃, b3) and (L4, a₄, b4) was calculated from the following expression.

ΔE=[(L3 - L4)²+(a₃ - a₄)²+(b3 - b4)²]¹ⁿ

ΔE was determined from the above expression, and the light resistance was evaluated according to the following determination criteria.

A: A case where ΔE is less than 10

B: A case where ΔE is 10 or more and less than 20

C: A case where ΔE is 20 or more

Storage Stability

As a compulsory test, the black dye inks prepared in Examples 1 to 10 and Comparative Examples 1 to 3 were stored for 4 weeks under the condition of a temperature of 60° C., and then the storage stability was evaluated.

The evaluation was carried out in terms of the two stages by denoting a case where the performance comparable to that of the black dye ink immediately after preparation is maintained as A and a case where the performance is deteriorated even in one item of the evaluation items (color tone, printing density, and light resistance) after the compulsory test, as B.

The results of Examples and Comparative Examples are shown in Table 1 below.

TABLE 1

| | Practical performance of black dye ink | | | |
|---|---|---|---|---|
| | Color tone | Printing density | Light resistance | Storage stability |
| Example 1 | C | B | B | A |
| Example 2 | B | B | B | A |
| Example 3 | A | A | A | A |
| Example 4 | A | A | A | A |
| Example 5 | A | A | A | A |
| Example 6 | A | A | A | A |
| Example 7 | A | A | A | A |
| Example 8 | A | B | A | A |
| Example 9 | A | A | B | A |
| Example 10 | A | A | B | A |
| Comparative | D | C | B | B |
| Example 1 | | | | |
| Comparative | D | B | C | B |
| Example 2 | | | | |
| Comparative | D | B | C | B |
| Example 3 | | | | |

### Example 11

100 parts by mass of the BLACK dye 1, 10 parts by mass of the additive (P-3), 1.50 parts by mass of chelating agent (EDTA), 1.0 part by mass of the preservative (PROXEL (registered trade name) XL2(s) manufactured by Lonza Group AG), and 1.00 part by mass of the buffer (NaHCO₃) were dissolved in 200.00 parts by mass of deionized water. Then, the pH was adjusted to 8.5 with a pH adjusting agent (a 10 mol/L sodium hydroxide aqueous solution), and the mixture was filtered under reduced pressure with a microfilter having an average pore diameter of 0.20 μm and washed with deionized water to prepare an aqueous dye solution 11 (1,000 parts by mass).

### Example 12

100 parts by mass of the BLACK dye 2, 10 parts by mass of the additive (P-3), 1.50 parts by mass of chelating agent (EDTA), 1.0 part by mass of the preservative (PROXEL (registered trade name) XL2(s) manufactured by Lonza Group AG), and 1.00 part by mass of the buffer (NaHCO₃) were dissolved in 200.00 parts by mass of deionized water. Then, the pH was adjusted to 8.5 with a pH adjusting agent (a 10 mol/L sodium hydroxide aqueous solution), and the mixture was filtered under reduced pressure with a microfilter having an average pore diameter of 0.20 μm and washed with deionized water to prepare an aqueous dye solution 12 (1,000 parts by mass).

### Example 13

100 parts by mass of the BLACK dye 3, 10 parts by mass of the additive (P-3), 1.50 parts by mass of chelating agent (EDTA), 1.0 part by mass of the preservative (PROXEL (registered trade name) XL2(s) manufactured by Lonza Group AG), and 1.00 part by mass of the buffer (NaHCO₃) were dissolved in 200.00 parts by mass of deionized water. Then, the pH was adjusted to 8.5 with a pH adjusting agent (a 10 mol/L sodium hydroxide aqueous solution), and the mixture was filtered under reduced pressure with a microfilter having an average pore diameter of 0.20 μm and washed with deionized water to prepare an aqueous dye solution 13 (1,000 parts by mass).

### Example 14

100 parts by mass of the BLACK dye 4, 10 parts by mass of the additive (P-3), 1.50 parts by mass of chelating agent (EDTA), 1.0 part by mass of the preservative (PROXEL (registered trade name) XL2(s) manufactured by Lonza Group AG), and 1.00 part by mass of the buffer (NaHCO₃) were dissolved in 200.00 parts by mass of deionized water. Then, the pH was adjusted to 8.5 with a pH adjusting agent (a 10 mol/L sodium hydroxide aqueous solution), and the mixture was filtered under reduced pressure with a microfilter having an average pore diameter of 0.20 μm and washed with deionized water to prepare an aqueous dye solution 14 (1,000 parts by mass).

### Comparative Example 4

An aqueous dye solution 4X (1,000 parts by mass) was prepared by the same treatment except that the preservative was not added to the aqueous dye solution of Example 11.

### Comparative Example 5

100 parts by mass of the BLACK dye 5, 10 parts by mass of the additive (P-3), 1.50 parts by mass of chelating agent (EDTA), 1.0 part by mass of the preservative (PROXEL (registered trade name) XL2(s) manufactured by Lonza Group AG), and 1.00 part by mass of the buffer (NaHCO₃) were dissolved in 200.00 parts by mass of deionized water. Then, the pH was adjusted to 8.5 with a pH adjusting agent (a 10 mol/L sodium hydroxide aqueous solution), and the mixture was filtered under reduced pressure with a microfilter having an average pore diameter of 0.20 μm and washed with deionized water to prepare an aqueous dye solution 5X (1,000 parts by mass).

### Antiseptic Properties

Using the aqueous dye solutions 11 to 14 prepared in Examples 11 to 14 and the aqueous dye solutions 4X and SX prepared in Comparative Examples 4 to 5, food stamps (for viable cell count and for fungi, respectively) for bacterial examination manufactured by Nissui Pharmaceutical Co., Ltd. were uncapped, and a sufficient aqueous dye solution was applied onto the surface of the agar culture medium. They were allowed to stand without capping for 10 hours to promote the adhesion of bacteria. Then, the food stamp for viable cell count was cultured in a constant-temperature tank at 36° C. for 2 days, and the food stamp for fungi was cultured in a constant-temperature tank at 23° C. for 5 days. By visualobservation, the evaluation was carried out in terms of the three stages according to the following criteria.

A . . . No fungi B . . . Although the outbreak of fungi is slightly confirmed, no tendency of growth is observed.

C . . . Outbreak of fungi is observed, and a tendency to grow is also observed.

### Storage Stability of Aqueous Dye Solution

As a compulsory test, the aqueous dye solutions 11 to 14 prepared in Examples 11 to 14 and the aqueous dye solutions 4X and 5X prepared in Comparative Examples 4 to 5 were stored for one week under the conditions of a temperature of 60° C. and a relative humidity of 50%, and then stored for 2 weeks under the conditions of a temperature at 60° C. and a relative humidity of 50°% o. Then, the storage stability (the presence or absence of filtration residue insoluble matter (precipitates) and change in liquid physical properties) was evaluated.

### Presence or Absence of Filtration Residue Insoluble Matter (Precipitate)

The aqueous dye solutions were filtered through a filter having a pore diameter of 0.2 pm and then visually observed. The evaluation was carried out in terms of the two stages by denoting a case where the dissolved state is maintained as A and a case where the precipitation or separation of foreign substances occurs as C.

### Changes in Liquid Physical Properties

The evaluation was carried out in terms of the three stages by denoting a case where the physical properties (absorptivity, viscosity, and surface tension) comparable to each aqueous solution immediately after the aqueous dye solution preparation are maintained as A, a case where after the compulsory test, one item is changed and the liquid physical property value is changed by ±3% or more as B, a case where after the compulsory test, two items or more are changed and the liquid physical property value is changed by ±3% or more as C. In addition, absorbance, viscosity, and surface tension were measured by the following methods, respectively.

### Absorbance

450 mg (0.45 g) of the aqueous dye solution was weighed and then diluted to 50 mL with deionized water, and 2 mL of the diluted solution was further diluted with deionized water to 100 mL. Then, the diluted aqueous solution was injected into a 1 cm×1 cm quartz cell, and the absorbance in a range of 250 nm to 900 nm was measured under an environment of 25° C. and a relative humidity of 50% by using a UV-Vis spectrography (UV-1800) manufactured by Shimadzu Corporation. After confirming the absorbance of Xrnax in the ultraviolet to visible range (250 to 700 nm), the change before and after the compulsory test was calculated.

### Viscosity

After weighing 1.5 mL of the aqueous dye solution using a viscometer (RE85) manufactured by TOKI SANGYO Co., Ltd., the measurement was carried out in an environment of 25° C. and a relative humidity of 50% to 70% in a measurement time of 2 minutes.

### Surface Tension

After weighing 5 mL of the aqueous dye solution using a surface tension meter (DY-200) manufactured by Kyowa Interface Science Co., Ltd., the measurement was carried out using a platinum plate at 25° C. and in an environment of a relative humidity of 30%.

### The Results are Shown in Table 2 Below.

Regarding antiseptic properties, the results of culturing the food stamp for viable cell count for 2 days in a constant-temperature tank at 36° C. are shown in the column of “For 2 days” of the exposure time, and the results of culturing the food stamp for fungi for 5 days in a constant-temperature tank at 23° C. are shown in the column of “For 5 days” of the exposure time.

TABLE 2

| | | | Presence or absence | | | |
|---|---|---|---|---|---|---|
| | Antiseptic | | of filtration residue | | Change in liquid | |
| | properties | | insoluble matter | | physical properties | |
| | For 2 | For 5 | For 1 | For 2 | For 1 | For 2 |
| | days | days | week | weeks | week | weeks |
| Example 11 | A | A | A | A | A | B |
| Example 12 | A | A | A | A | A | A |
| Example 13 | A | A | A | A | A | A |
| Example 14 | A | A | A | A | A | A |
| Comparative | B | C | A | C | B | C |
| Example 4 | | | | | | |
| Comparative | A | A | A | A | B | C |
| Example 5 | | | | | | |

What is claimed is:

1. A dye ink composition comprising:
a phthalocyanine compound represented by the following General Formula (I), 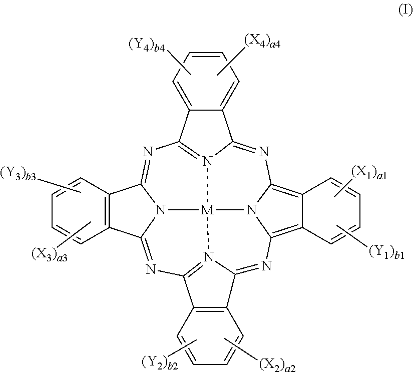
wherein, in the General Formula (I),
X₁, X₂, X₃, and X₄ each independently represent —N═N—Cp,
Cp represents an organic group,
Y₁, Y₂, Y₃, and Y₄ each independently represent a substituent,
at least one of Xi, X₂, X₃, X₄, Y₁, Y₂, Y.;, or Y₄ contains an ionic hydrophilic group,
a1, a2, a3, a4, b1, b2, b3, and b4 each independently represent an integer of 0 to 4,
a1 +b1, a2+b2, a3+b3, and a₄+b4 are each independently an integer of 0 to 4,
all of a₁, a₂, a₃, and a₄ are not 0 at the same time, and
M represents a hydrogen atom, a metal element, a metal oxide, a metal hydroxide, or a metal halide.

2. The dye ink composition according to claim 1,
wherein the phthalocyanine compound is a phthalocyanine compound represented by the following General Formula (11), 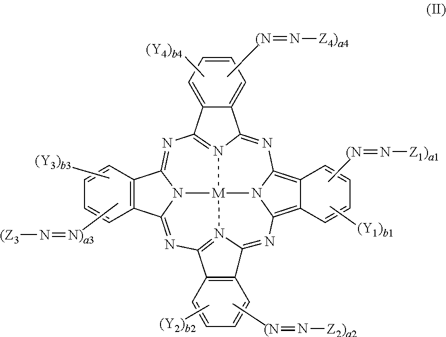
wherein, in the General Formula (II),
Z₁, Z₂, Z₃, and Z₄ each independently represent a heterocyclic group, the heterocyclic group may have a substituent,
Y₁, Y₂, Y₃, and Y₄ each independently represent a substituent,
a1, a2, a3, a4, b1, b2, b3, and b4 each independently represent an integer of 0 to 4,
a1 +b1, a2+b2, a₃+b3, and a₄+b4 are each independently an integer of 0 to 4,
all of a1, a2, a3, and a4 are not 0 at the same time,
M represents a hydrogen atom, a metal element, a metal oxide, a metal hydroxide, or a metal halide, and
at least one of Z₁, Z₂, Z₃, Z₄, Y₁, Y₂, Y₃, or Y₄ contains an ionic hydrophilic group.

3. The dye ink composition according to claim 2,
wherein the phthalocyanine compound is a phthalocyanine compound represented by the following General Formula (III), 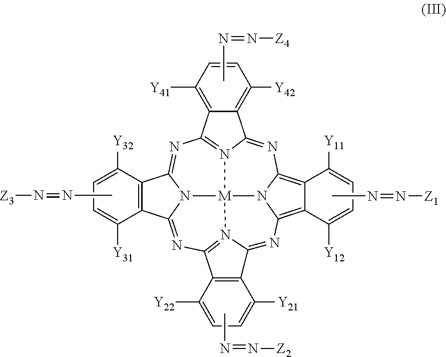
wherein, in the General Formula (III),
Z₁, Z₂, Z₃, and Z₄ each independently represent a heterocyclic group, the heterocyclic group may have a substituent,
M represents a hydrogen atom, a metal element, a metal oxide, a metal hydroxide, or a metal halide,
Y₁₁, Y₁₂, Yz1, Y₁₂, Y₃₁, Y₃₂, Y₄₁ and Y₄z each independently represent a hydrogen atom or a substituent, and
at least one of Z₁, Z₂, Z₃, Z_{4,} ^{Y}₁₁, Y₁₂, Y_{21,} ^{Y}₂₂, Y₃₁, Y₃₂, Y₄₁, or Y₄₂ contains an ionic hydrophilic group.

4. The dye ink composition according to claim 3,
wherein the phthalocyanine compound is a phthalocyanine compound represented by the following General Formula (IV), 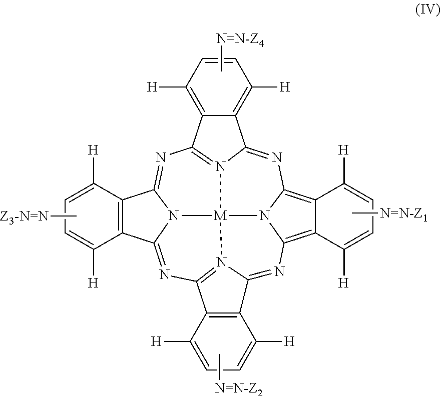
wherein, in the General Formula (N),
Z₁, Z₂, Z₃, and Z₄ each independently represent a heterocyclic group, the heterocyclic group may have a substituent,
M represents a hydrogen atom, a metal element, a metal oxide, a metal hydroxide, or a metal halide, and
at least one of Z₁, Z₂, Z₃, or Za contains an ionic hydrophilic group.

5. The dye ink composition according to claim 1,
wherein the ionic hydrophilic group is at least one of —SO₃R, —CO₂R, or —PO(OR)2,
R represents a hydrogen atom or a counter cation, and
two R's of -PO(OR)2 may be the same or different from each other.

6. The dye ink composition according to claim 1,
wherein a content of the phthalocyanine compound is 1.0°% o to 6.5% by mass with respect to a total mass of the dye ink composition.

7. The dye ink composition according to claim 1, further comprising:
a compound represented by the following General Formula (P), 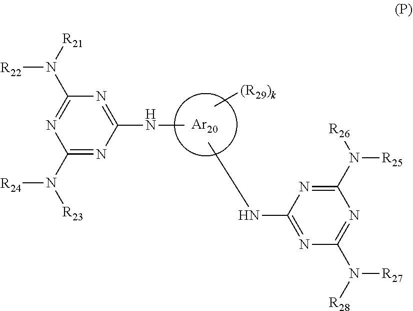
wherein, in the General Formula (P), Arzo represents a benzene ring or a naphthalene ring; R₂₁ to R₂₈ each independently represent a hydrogen atom or a substituent; Rn and R₂₂ may be bonded to each other to form a ring; R₂₃ and Rea may be bonded to each other to form a ring; R₂₅ and R₂₆ may be bonded to each other to form a ring; R₂₇ and R₂₈ may be bonded to each other to form a ring; R₂₉ represents a substituent; in a case where Arlo represents a benzene ring, k represents an integer of 0 to 4; in a case where Ara) represents a naphthalene ring, k represents an integer of 0 to 6; in a case where a plurality of R₂₉'s are present, the plurality of R₂₉'s may be the same or different from each other; in a case where a plurality of R₂₉'s are present, the plurality of R₂₉'s may be bonded to each other to form a ring; and at least one of R₂₁ to R₂₉ has a hydrophilic group.

8. The dye ink composition according to claim 7,
wherein a content of the compound represented by the General Formula (P) is 0.5% to 3.0% by mass with respect to a total mass of the dye ink composition.

9. The dye ink composition according to claim 1, further comprising a toning dye.

10. The dye ink composition according to claim 1, further comprising a chelating agent.

11. The dye ink composition according to claim 1, further comprising a preservative.

12. A black dye ink comprising the dye ink composition according to claim 1.

13. An ink jet recording dye ink comprising:
the dye ink composition according to claim 1.

14. An ink jet recording dye ink comprising:
the black dye ink according to claim 12.

15. An ink jet recording method comprising jetting the ink jet recording dye ink according to claim 13 using an ink jet method-type recording head.

16. An ink jet recording method comprising jetting the ink jet recording dye ink according to claim 14 using an ink jet method-type recording head.

17. An aqueous dye solution comprising:
a preservative,
wherein the aqueous dye solution contains a phthalocyanine compound represented by the following General Formula (I), and
a content of the phthalocyanine compound represented by the General Formula (I) is 8% to 15% by mass with respect to a total mass of the aqueous dye solution, 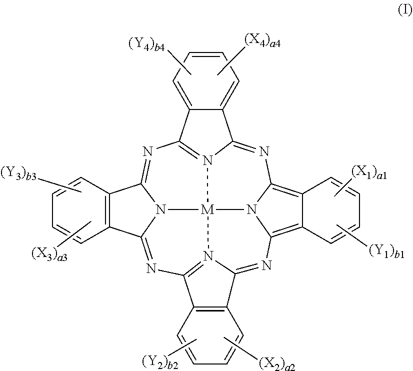
wherein, in the General Formula (I),
X₁, X₂, X₃, and Xa each independently represent —N═N—Cp,
Cp represents an organic group,
Y₁, Y₂, Y₃, and Y₄ each independently represent a substituent,
at least one of X₁, X₂, X₃, X₄, Yt, Y₂, Y₃, or Y₄ contains an ionic hydrophilic group,
a1, a2, a3, a4, b1, b2, b3, and b4 each independently represent an integer of 0 to 4,
a1 +b 1, a2+b2, a3+b3, and a4+b4 are each independently an integer of 0 to 4,
all of a1, a2, a3, and a4 are not 0 at the same time, and
M represents a hydrogen atom, a metal element, a metal oxide, a metal hydroxide, or a metal halide.

18. The aqueous dye solution according to claim 17, further comprising a toning dye.
